(19) United States
(12) Patent Application Publication
(10) Pub. No.: US 2026/0314988 A1
(43) Pub. Date: Oct. 15, 2026

(54) MENTAL DISORDER TREATMENT

(71) Applicant: GrayMatters Health Ltd., Haifa (IL)

(72) Inventors: Oded Shlomo KRAFT-OZ, Haifa (IL); Ran COHEN, Tel-Aviv (IL); Shai ATTIA, Shimshit (IL); Dmitri LAVRO, Kfar Vradim (IL); Yaakov STERN, Sde Yaakov (IL)

(73) Assignee: GrayMatters Health Ltd., Haifa (IL)

(21) Appl. No.: 19/322,788

(22) Filed: Sep. 9, 2025

Related U.S. Application Data
(63) Continuation of application No. PCT/IL2024/050247, filed on Mar. 7, 2024.
(60) Provisional application No. 63/451,015, filed on Mar. 9, 2023.

(51) Int. Cl. A61B 5/375

(52) U.S. Cl. CPC A61B 5/375

(57) ABSTRACT

A method for delivery of a neurofeedback (NF) process to a subject, including: teaching a subject to modify activity of at least one specific brain region, by performing at least one NF session of the treatment, the performing comprises: presenting to the subject a sensory indication, instructing the subject to modify the sensory indication by performing a specific activity, monitoring changes in activity of the at least one brain region during the presenting, and providing to the subject a feedback signal indicating the monitored changes, during said presenting; delivering to the subject after said NF session is completed, a maintenance session of the NF process, wherein the maintenance session comprises presenting to said subject a trigger selected to evoke said sensory indication used during the NF process session, and instructing the subject to perform the specific activity used during the NF process session in response to the presenting.

### RELATED APPLICATIONS

This application is a Continuation of PCT Patent Application No. PCT/IL2024/050247 having International filing date of Mar. 7, 2024, which claims the benefit of priority under 35 USC § 119(e) of U.S. Provisional Patent Application No. 63/451,015, filed on Mar. 9, 2023. The contents of the above applications are all incorporated by reference as if fully set forth herein in their entirety.

### FIELD AND BACKGROUND OF THE INVENTION

The present invention, in some embodiments thereof, relates to treatment of mental disorders and, more particularly, but not exclusively, to treatment of mental disorders using biofeedback, for example neurofeedback.

### SUMMARY OF THE INVENTION

Some examples of some embodiments of the invention are listed below (an embodiment of the invention may include features from more than one example and/or fewer than all features of an example):
Example 1. A method for delivery of a neurofeedback (NF) treatment to a subject, comprising: teaching a subject to modify activity of at least one specific brain region by performing a NF session of said treatment, said performing comprises:
presenting to said subject a sensory indication, instructing said subject to modify said sensory indication by performing a specific activity, monitoring changes in activity of said at least one brain region during said presenting, and providing to said subject a feedback signal indicating said monitored changes during said presenting;
delivering to said subject a maintenance session of said treatment at least 2 hours following said teaching, said maintenance session is configured to evoke at least part of said NF session in said subject, said delivering comprises:
presenting to said subject an indication configured to evoke said sensory indication used during said teaching, and instructing said subject to perform said specific activity used in said teaching in response to said presenting.

Example 2. A method according to example 1, wherein said teaching is performed in a treatment center, and wherein said delivering is performed outside said treatment center.

Example 3. A method according to example 2, wherein said treatment center comprises a clinic.

Example 4. A method according to any one of the previous examples, wherein said teaching comprises measuring electrical signals from said subject brain during said teaching, and wherein said monitoring comprises monitoring said changes in activity of said at least one brain region based on said measured electrical signals.

Example 5. A method according to any one of the previous examples, wherein said delivering said maintenance session does not include measuring of electrical signals from said subject brain.

Example 6. A method according to any one of examples 4 or 5, wherein said electrical signals comprise electroencephalogram (EEG) signals.

Example 7. A method according to any one of the previous examples, wherein said teaching comprising selecting said specific activity from two or more activities used in said teaching.

Example 8. A method according to any one of the previous examples, wherein said delivering is according to a predetermined schedule of said treatment.

Example 9. A method according to any one of examples 1 to 7, comprising receiving an input from said subject following said teaching, and wherein said delivering is in response to said received input.

Example 10. A method according to example 9, wherein said input comprises information that said subject currently experiences or is expecting to experience at least one of, cognitive difficulties, behavioral difficulties and/or mental difficulties.

Example 11. A method according to any one of the previous examples, comprising repeating said performing in at least one additional NF session, and wherein said delivering is performed during an interval period between said at least one NF session and said at least one additional NF session.

Example 12. A method according to any one of the previous examples, comprising transmitting an indication to a supervisor of said treatment after completion of said delivering of said maintenance session.

Example 13. A method according to any one of the previous examples, comprising:
diagnosing said subject with a mental or a brain disorder prior to said teaching and/or identifying that said subject presents at least one symptom of a mental or brain disorder prior to said teaching

Example 14. A method according to example 13, wherein said mental or brain disorder comprises a stress disorder, post-traumatic stress disorder (PTSD), depression, Attention deficit hyperactivity disorder (ADHD), substance use disorder (SUD), dementia, Alzheimer's disease, Mild Cognitive Impairment, Autism.

Example 15. A method according to example 14, comprising adjusting a timing for performing said teaching and/or of said delivering, according to said mental or brain disorder, or according to said at least one symptom.

Example 16. A method according to any one of examples 14 or 15, comprising modifying said sensory indication, and/or said specific activity to be performed by said subject according to said mental or brain disorder or according to said at least one symptom.

Example 17. A method according to any one of the previous examples, wherein said sensory indication comprises an audio and/or a visual indication.

Example 18. A method for delivery of a neurofeedback (NF) treatment to a subject, comprising:
teaching a subject to modify activity of at least one specific brain region by performing at least one NF session of said treatment, said performing comprises:
presenting to said subject a sensory indication, instructing said subject to modify said sensory indication by performing a specific activity, monitoring changes in activity of said at least one brain region during said presenting, and providing to said subject a feedback signal indicating said monitored changes during said presenting;
determining a state of said subject at least 1 hour from ending said performing of said at least one NF session, wherein said state of said subject comprises at least one of, a mental state, a behavioral state, and/or a cognitive state of said subject.

Example 19. A method according to example 18, comprising:
scheduling at least one additional NF session for repeating said teaching, according to said determined subject state.

Example 20. A method according to example 19, comprising:
modifying according to said determined state at least one of, said sensory indication, said specific activity, instructions delivered to said subject during said teaching, and/or said specific activity to be applied by said subject.

Example 21. A method according to any one of examples 18 to 20, comprising:
performing or scheduling, a maintenance session of said NF treatment, according to said determined state, wherein said maintenance session comprises presenting to said subject an indication configured to evoke said sensory indication used during said teaching, and instructing said subject to perform said specific activity used in said teaching.

Example 22. A method according to any one of examples 18 to 21, comprising:
transmitting an alert indication to an expert or a remote server with information on said determined subject state.

Example 23. A method according to any one of examples 18 to 22, comprising:
transmitting suggestions to said subject or to an expert monitoring said treatment according to said determined subject state, wherein said suggestions include suggestions to initiate or to stop or to modify at least one additional treatment.

Example 24. A method according to example 23, wherein said at least one additional treatment comprises at least one of, a drug treatment, a psychological treatment, a behavioral treatment.

Example 25. A method according to any one of examples 18 to 24, wherein said determining comprises determining said subject state based on results of at least one assessment questionnaire.

Example 26. A method according to example 25, wherein said at least one assessment questionnaire comprises at least one of, a Clinician-Administered PTSD Scale for DSM-5 (CAPS-5) and/or parameters or domains of the CAPS-5 or derivatives thereof, a PTSD Checklist for DSM-5 (PCL-5) or derivatives thereof, a Patient Health Questionnaire-9 (PHQ-9) or derivatives thereof, a Hamilton Depression Rating Scale or derivatives thereof, a Snaith-Hamilton Pleasure Scale (SHAPS) or derivatives thereof, a Montgomery-Asberg Depression Rating Scale (MADRS) or derivatives thereof, a Patient Health Questionnaire (PHQ) or derivatives thereof, a Clinical Global Impression (CGI) or derivatives thereof, an Adult Attention deficit hyperactivity disorder (ADHD) ADHD Investigator Symptom Rating Scale (AISRS) or derivatives thereof, an Adult ADHD Self-Report Scale (ASRS) or derivatives thereof, a Test of Variables of Attention (TOVA) or derivatives thereof, a Patient Health Questionnaire (PHQ) or derivatives thereof, an Alzheimer's Disease Assessment Scale-Cognitive Subscale (ADAS-Cog) or derivatives thereof, a mini-mental state examination (MMSE) or Folstein test or derivatives thereof.

Example 27. A method according to any one of examples 18 to 26, wherein said determining comprises determining said subject state based on measurements of at least one physiological parameter comprises at least one of, heart rate, blood pressure, skin conductance, eye movement, pupil dilation.

Example 28. A method according to any one of examples 18 to 27, comprising:
diagnosing said subject with a mental or a brain disorder prior to said teaching and/or identifying that said subject presents at least one symptom of a mental or brain disorder prior to said teaching.

Example 29. A method according to example 28, wherein said mental or brain disorder comprises at least one of, a stress disorder, post-traumatic stress disorder (PTSD), depression, Attention deficit hyperactivity disorder (ADHD), substance use disorder (SUD), dementia, Alzheimer's disease, Mild Cognitive Impairment, Autism.

Example 30. A method according to example 29, comprising adjusting a timing for said performing of said at least one NF session or a timing for performing at least one additional NF session, according to said diagnosed mental or brain disorder, or according to said at least one symptom.

Example 31. A method according to any one of examples 29 or 30, comprising adjusting said sensory indication, and/or said specific activity to be performed by said subject, according to said mental or brain disorder or according to said at least one symptom.

Example 32. A method for delivery of a neurofeedback (NF) treatment to a subject, comprising:
delivering a NF treatment to a subject for teaching said subject to modulate an activity of at least one specific brain region, wherein said delivering comprises delivering two or more sessions of said NF treatment with an interval of at least 1 day therebetween;
generating during said delivering of said NF treatment, a progress indication regarding a progress in an ability of said subject to modulate an activity of said at least one specific brain region, and an subject state indication regarding a mental and/or behavioral state of said subject;
displaying said progress indication and said subject state indication.

Example 33. A method according to example 32, comprising modifying said NF treatment delivered to said subject based on said generated progress indication and/or based on said subject state indication.

Example 34. A method according to example 32, comprises determining a relation between said progress indication and said subject state indication, and modifying said NF treatment delivered to said subject based on said determined relation.

Example 35. A method according to any one of examples 33 or 34, wherein said modifying said NF treatment comprises at least one of, prolonging said NF treatment, shortening said NF treatment, stopping said NF treatment, modifying a sensory indication presented to said subject during said NF treatment, modifying instructions provided to said subject during said NF treatment. Example 36. A method according to any one of examples 32 to 35, comprising receiving information about said mental and/or behavioral state of said subject, and wherein said subject state indication is generated based on said received information.

Example 37. A method according to example 36, wherein said received information comprises results of assessment questionnaires.

Example 38. A method according to any one of examples 36 or 37, wherein said information comprises input received from said subject and/or an expert.

Example 39. A method according to any one of examples 32 to 38, wherein said generating comprises generating a trend of a change in said progress indication, and a trend of a change in said subject state indication, over a specific time period, and wherein said displaying comprises displaying said progress indication trend and said subject state trend.

Example 40. A method according to any one of examples 32 to 39, wherein said generating comprises generating a predicted progress indication and a predicted subject state indication, and wherein said displaying comprises displaying said predicted progress indication trend and said predicted subject state indication.

Example 41. A method according to any one of examples 32 to 40, wherein said displaying comprises displaying said progress indication and said subject state indication in a shared visual indication.

Example 42. A method for identifying an electrode with a target signal quality, comprising: recording electrical signals by at least two electrodes positioned on a subject body;

identifying an electrode of said at least two electrodes which generates an electrical signal with a selected intensity value;
setting a target range of values relative to said selected intensity value;
identifying at least one additional electrode of said at least two electrodes which generates a signal having an intensity value within said target range of values;
determining that said intensity value of said signal of said identified at least one additional electrode, is higher than a predetermined value;
delivering a human detectable indication based on said determining.

Example 43. A method according to example 42 wherein said delivering comprises delivering instructions how to improve signal quality from said identified at least one additional electrode if said determining indicates that said intensity value is lower than said predetermined value.

Example 44. A neurofeedback (NF) system for delivery of a NF treatment which includes at least one NF treatment session and at least one maintenance session to a subject, comprising:
(a) a NF device configured to deliver said at least one NF treatment session to said subject for teaching said subject to modulate an activity of at least one specific brain region, comprising:
a memory storing at least one protocol including at least one sensory indication, and instructions to be delivered to said subject by said NF device;
a user interface;
a control circuitry, wherein said control circuitry is configured to:
deliver said at least one sensory indication to said subject using said user interface, instruct said subject to modulate said delivered at least one visual and/or audio interface by performing at least one activity;
receive electrical signals from one or more electrodes positioned on a body of said subject, measure using said electrical signals indications regarding an activity or changes thereof of at least one specific brain region, and
provide a feedback signal to said subject during said delivery of said at least one first visual and/or audio interface, according to said measured indications;
(b) a personal device of said subject in communication directly or indirectly with said NF device, configured to deliver at least one maintenance session of said NF treatment configured to evoke at least part of said at least one NF treatment session in said subject, said personal device comprising:
a memory storing a maintenance protocol which includes at least one indication configured to evoke said sensory indication delivered by said NF device during said at least one NF treatment session;
a user interface;
a control circuitry, wherein said control circuitry is configured to deliver using said user interface, said at least one indication and instructions to perform said at least one activity as performed in said at least one NF treatment session.

Example 45. A system according to example 44, wherein said personal device and said NF device each comprise a communication circuitry, and wherein said personal device is in communication directly or indirectly with said NF device using said communication circuitry.

Example 46. A system according to any one of examples 44 or 45, wherein said memory of NF device comprise information on a state of said subject.

Example 47. A system according to example 46, wherein said control circuitry of said NF device generates, selects and/or modifies said at least one sensory indication according to said information stored in a memory of said NF device.

Example 48. A system according to any one of examples 46 or 47, wherein said control circuitry of said NF device selects or replaces said at least one protocol for teaching said subject to modulate a different specific brain region, according to said stored information.

Example 49. A system according to any one of examples 45 to 48, wherein said information comprises information that said subject is diagnosed with a mental or a brain disorder, or presents a symptom of a mental or a brain disorder.

Example 50. A system according to example 49, wherein said mental or brain disorder comprises at least one of, a stress disorder, post-traumatic stress disorder (PTSD), depression, Attention deficit hyperactivity disorder (ADHD), substance use disorder (SUD), dementia, Alzheimer's disease, Mild Cognitive Impairment, Autism.

Example 51. A system according to any one of examples 46 to 50, wherein said at least one indication is a modification of said at least one sensory indication.

Example 52. A system according to any one of examples 44 or 45, comprising:
a server in communication with said NF device and said personal device, comprising: a memory which stores at least one NF treatment protocol and wherein said at least one protocol stored in said NF device memory and said maintenance protocol stored in said personal device memory are part of said NF treatment protocol.

Example 53. A system according to example 52, wherein said server stores information about a state of said subject and/or information that said subject expects to have difficulties in daily activities.

Example 54. A system according to example 53 wherein said information about said state of said subject comprises information on at least one of, a mental state, a cognitive state, a behavioral state.

Example 55. A system according to any one of examples 53 or 54, wherein said information stored in said server comprises information that said subject is diagnosed with a mental or a brain disorder, or presents a symptom of a mental or a brain disorder.

Example 56. A system according to example 55, wherein said mental or brain disorder comprises at least one of, a stress disorder, post-traumatic stress disorder (PTSD), depression, Attention deficit hyperactivity disorder (ADHD), substance use disorder (SUD), dementia, Alzheimer's disease, Mild Cognitive Impairment, Autism.

Example 57. A system according to any one of examples 53 to 56, wherein said server is configured to at least one of, reschedule said at least one NF treatment session and/or said at least one maintenance session, schedule a meeting with an expert, and/or deliver instructions to said subject via said personal device, according to said stored information.

Example 58. A system according to example 57, wherein said instructions comprise perform at least one maintenance session.

Example 59. A system according to any one of examples 57 or 58, wherein said instructions comprise instructions to initiate, modify or stop at least one additional treatment.

Example 60. A system according to example 59, wherein said at least one additional treatment comprises at least one of, a drug treatment, a psychological treatment, and/or a behavioral treatment.

Example 61. A system according to any one of examples 53 to 60, wherein said server modifies at least one parameter of said NF treatment session according to said stored information.

Example 62. A system according to example 61, wherein said at least one parameter comprises at least one of, duration of at least one NF session to be delivered by said NF device, duration of a maintenance session to be delivered by said personal device, time between said at least one NF session and said at least one maintenance session, time between two consecutive NF sessions, number of NF sessions to be delivered to said subject, number of maintenance sessions to be delivered to said subject, content of said at least one maintenance session, content of said at least one NF session.

Example 63. A system according to any one of examples 53 to 62, wherein said information is received from said NF device and/or said personal device.

Example 64. A system, comprising:
a device configured to deliver a neurofeedback (NF) treatment to a subject for teaching said subject to modulate an activity of at least one specific brain region, comprising:
a memory storing at least one protocol including at least one sensory indication, and instructions to be delivered to said subject by said NF device, wherein said memory further comprises information regarding a mental state of said subject;
a user interface;
a control circuitry, wherein said control circuitry is configured to:
deliver said at least one visual and/or audio interface to said subject using said user interface, instruct said subject to modulate said delivered at least one visual and/or audio interface by performing at least one activity;
receive electrical signals from one or more electrodes positioned on a body of said subject, measure using said electrical signals indications regarding an activity or changes thereof of at least one specific brain region, and
provide a feedback signal to said subject during said delivery of said at least one first visual and/or audio interface, according to said measured indications;
wherein said control circuitry is further configured to generate based on said received electrical signals a progress indication which indicates a progress of said subject to modulate an activity of said at least one brain region, and a subject state indication based on said information stored in said memory.

Example 65. A system according to example 64, comprising a supervisor interface functionally coupled to said device and configured to generate a human detectable indication, wherein said control circuitry signals said supervisor interface to display said progress indication and said subject state indication.

Example 66. A system according to example 65, wherein said control circuitry is configured to determine a relation between said progress indication and said subject state indication, and to signal said supervisor interface to display said determined relation.

Example 67. A system according to any one of examples 65 or 66, wherein said control circuitry is configured to generate a trend of a change of said progress indication, and a trend of a change of said subject state indication over a specific time period and/or predictions thereof, and to signal said supervisor interface to display said generated said trend of said change of said progress indication and said trend of said change of said subject state indication, and/or of said predictions thereof.

Below are some additional examples of some embodiments of the invention (an embodiment of the invention may include features from more than one example and/or fewer than all features of an example):
Example 1. A method for delivery of a neurofeedback (NF) process to a subject, comprising:
teaching a subject to modify activity of at least one specific brain region or at least one specific brain network, by performing at least one NF session of said treatment, said performing comprises:
presenting to said subject a sensory indication, instructing said subject to modify said sensory indication by performing a specific activity, monitoring changes in activity of said at least one brain region or said at least one brain network during said presenting, and providing to said subject a feedback signal indicating said monitored changes during said presenting;
delivering to said subject after said NF session is completed, a maintenance session of said NF process, wherein said maintenance session comprises presenting to said subject a trigger selected to evoke said sensory indication used during said NF process session, and instructing said subject to perform said specific activity used during said NF process session in response to said presenting.

Example 2. A method according to example 1, wherein said teaching is performed in a therapy center, and wherein said delivering is performed outside said therapy center, wherein said therapy center comprises a clinic.

Example 3. A method according to any one of the previous examples, wherein said teaching comprises measuring electrical signals from said subject brain during said teaching, and wherein said monitoring comprises monitoring said changes in activity of said at least one brain region or said at least one brain network based on said measured electrical signals.

Example 4. A method according to example 3, wherein said delivering said maintenance session does not include measuring of electrical signals from said subject brain.

Example 5. A method according to any one of examples 3 or 4, wherein said electrical signals comprise electroencephalogram (EEG) signals.

Example 6. A method according to any one of the previous examples, wherein said teaching comprises teaching said subject to modify said activity of said at least one specific brain region or said at least one specific brain network by performing at least two NF sessions of said process separated by an interval of at least 24 hours, and wherein said delivering comprises delivering said maintenance session to said subject during said interval.

Example 7. A method according to any one of examples 1 to 5, comprising receiving an input from said subject following said teaching, and wherein said delivering is in response to said received input.

Example 8. A method according to example 7, wherein said input comprises information that said subject currently experiences or is expected to experience at least one of, cognitive difficulties, behavioral difficulties and/or mental difficulties.

Example 9. A method according to any one of the previous examples, comprising: diagnosing said subject with a mental or a brain disorder prior to said teaching and/or identifying that said subject presents at least one symptom of a said mental or brain disorder prior to said teaching.

Example 10. A method according to example 9, wherein said mental or brain disorder comprises a stress disorder, post-traumatic stress disorder (PTSD), depression, Attention deficit hyperactivity disorder (ADHD), substance use disorder (SUD), dementia, Alzheimer's disease, Mild Cognitive Impairment, Autism.

Example 11. A method according to example 10, comprising adjusting a timing for performing said teaching and/or of said delivering, according to a severity of said mental disorder or said severity of said at least one symptom.

Example 12. A method according to any one of the previous examples, wherein said trigger has at least a partial similarity with said sensory indication presented during the NF session to said subject.

Example 13. A method according to any one of the previous examples, wherein said at least one specific brain region comprises an amygdala and/or a ventral striatum.

Example 14. A method according to any one of the previous examples, wherein said at least one specific brain network comprises a limbic network and/or an award network.

Example 15. A method for delivery of a neurofeedback (NF) process to a subject, comprising: teaching a subject to modify activity of at least one specific brain region or at least one specific brain network, by performing at least one NF session of said process, said performing comprises:
presenting to said subject a sensory indication, instructing said subject to modify said sensory indication by performing a specific activity, monitoring changes in activity of said at least one brain region or said at least one brain network during said presenting, and providing to said subject a feedback signal indicating said monitored changes during said presenting;
determining a state of said subject after completing said at least one NF session, wherein said state of said subject comprises at least one of, a mental state, a behavioral state, and/or a cognitive state of said subject.

Example 16. A method according to example 15, comprises receiving information about a state of said subject after completing said at least one NF session, wherein said information comprises results of at least one assessment questionnaire, self-reported information, and/or input from an expert about the state of the subject.

Example 17. A method according to example 16, wherein said at least one assessment questionnaire comprises at least one of, a Clinician-Administered PTSD Scale for DSM-5 (CAPS-5) and/or parameters or domains of the CAPS-5 or derivatives thereof, a PTSD Checklist for DSM-5 (PCL-5) or derivatives thereof, a Patient Health Questionnaire-9 (PHQ-9) or derivatives thereof, a Hamilton Depression Rating Scale or derivatives thereof, a Snaith-Hamilton Pleasure Scale (SHAPS) or derivatives thereof, a Montgomery-Asberg Depression Rating Scale (MADRS) or derivatives thereof, a Patient Health Questionnaire (PHQ) or derivatives thereof, a Clinical Global Impression (CGI) or derivatives thereof, an Adult Attention deficit hyperactivity disorder (ADHD) ADHD Investigator Symptom Rating Scale (AISRS) or derivatives thereof, an Adult ADHD Self-Report Scale (ASRS) or derivatives thereof, a Test of Variables of Attention (TOVA) or derivatives thereof, a Patient Health Questionnaire (PHQ) or derivatives thereof, an Alzheimer's Disease Assessment Scale-Cognitive Subscale (ADAS-Cog) or derivatives thereof, a mini-mental state examination (MMSE) or Folstein test or derivatives thereof.

Example 18. A method according to any one of examples 15 to 17, comprising scheduling at least one additional NF session for repeating said teaching if said determined state of said subject is worse than a target state.

Example 19. A method according to any one of examples 15 to 18, comprising modifying according to said determined state at least one of, said sensory indication, said specific activity, instructions delivered to said subject during said teaching, and/or said specific activity to be applied by said subject, if said determined state of said subject is worse than a target state.

Example 20. A method according to any one of examples 15 to 19, comprising:
performing or scheduling, a maintenance session of said NF treatment, according to said determined state, wherein said maintenance session comprises presenting to said subject an indication configured to evoke said sensory indication used during said teaching, and instructing said subject to perform said specific activity used in said teaching.

Example 21. A method according to any one of examples 15 to 20, comprising:
transmitting suggestions to said subject or to an expert monitoring said treatment according to said determined subject state, wherein said suggestions include suggestions to initiate or to stop or to modify at least one additional treatment, wherein said at least one additional treatment comprises at least one of, a drug treatment, a psychological treatment, and a behavioral treatment.

Example 22. A method according to any one of examples 15 to 21, comprising:
diagnosing said subject with a mental disorder or at least one symptom thereof, prior to said teaching, wherein said mental disorder comprises at least one of, a stress disorder, post-traumatic stress disorder (PTSD), depression, Attention deficit hyperactivity disorder (ADHD), substance use disorder (SUD), dementia, Alzheimer's disease, Mild Cognitive Impairment, Autism.

Example 23. A method for delivery of a neurofeedback (NF) treatment to a subject, comprising:
delivering a NF treatment to a subject for teaching said subject to modulate an activity of at least one specific brain region, wherein said delivering comprises delivering two or more sessions of said NF treatment with an interval of at least 1 day therebetween;
generating during said delivering of said NF treatment, a progress indication regarding a progress in an ability of said subject to modulate an activity of said at least one specific brain region, and a subject state indication regarding a mental and/or behavioral state of said subject;
displaying said progress indication and said subject state indication in a shared visual interface.

Example 24. A method according to example 23, wherein said progress indication and said subject state indication are displayed side by side in said shared visual interface.

Example 25. A method according to any one of examples 23 or 24, comprising modifying said NF treatment delivered to said subject based on said generated progress indication and/or based on said subject state indication.

Example 26. A method according to any one of examples 23 to 25, comprising determining a correlation between said progress indication and said subject state indication, and modifying said NF treatment delivered to said subject based on said determined correlation.

Example 27. A method according to any one of examples 23 to 26, wherein said generating a progress indication comprises calculating a progress score indicting said progress of said subject, and at least one subject state score indicating a state of said subject, and wherein said displaying comprises displaying said progress score and said at least one subject state score in said shared visual interface.

Example 28. A method according to example 27, wherein said displaying comprises displaying changes in said progress score and changes in said subject state score in said shared visual interface. Example 29. A method according to any one of examples 23 to 28, wherein said subject state comprises at least one of, mental state, cognitive state, and functional state, of the subject.

Example 30. A method according to any one of examples 23 to 29, comprising receiving information about said state of said subject, and wherein said subject state indication is generated based on said received information.

Example 31. A method according to example 30, wherein said received information comprises results of at least one assessment questionnaire, input received from said subject and/or input received from an expert.

Example 32. A method according to any one of examples 23 to 31, wherein said generating comprises generating a predicted progress indication and a predicted subject state indication, and wherein said displaying comprises displaying said predicted progress indication trend and said predicted subject state indication.

Example 33. A method for determining that an electrode has a target signal quality, comprising:
recording electrical signals by at least two electrodes coupled to a subject body;
processing said recorded electrical signals by:
identifying at least one electrode of said at least two electrodes which generates an electrical signal with a target peak to peak value;
setting a target range of values relative to said identified peak to peak value;
determining that a signal recorded by at least one additional electrode of said at least two electrodes has a target signal quality if said signal recorded by said at least one additional electrode has a peak to peak value within said set target range of values and if an amplitude of said signal is within a predetermined range of values or higher than a predetermined amplitude value;
delivering a human detectable indication based on said determining.

Example 34. A method according to example 33, wherein said recording said processing, said determining and said delivering are performed prior to and/or during biofeedback treatment.

Example 35. A neurofeedback (NF) system for delivery of a NF treatment which includes at least one NF treatment session and at least one maintenance session, to a subject, comprising:
(a) a NF device configured to deliver said at least one NF treatment session to said subject, wherein during said at least one NF treatment session at least one sensory indication is delivered to said subject with instructions to modulate said delivered at least one sensory indication by performing at least one specific activity;
(b) a personal device of said subject in communication with said NF device, configured to deliver at least one maintenance session of said NF treatment, wherein said at least one maintenance session is configured to evoke at least part of said at least one NF treatment session in said subject, said personal device comprising:
a memory storing a maintenance protocol which includes at least one trigger configured to evoke said sensory indication delivered by said NF device during said at least one NF treatment session; a user interface;
a control circuitry, wherein said control circuitry is configured to deliver using said user interface, said at least one trigger and instructions to perform said at least one specific activity used during said at least one NF treatment session.

Example 36. A system according to example 35, wherein said sensory indication comprises a visual and/or an audio interface, and wherein said trigger has at least a partial similarity with said visual and/or audio interface.

Example 37. A system according to any one of examples 35 or 36, wherein said memory of said personal device comprises a timing schedule for performing said at least one maintenance session, and wherein said control circuitry of said personal device is configured to deliver said at least one maintenance session according to said timing schedule.

Example 38. A system according to any one of examples 35 to 37, wherein said NF device comprises
a memory storing at least one NF treatment protocol with said at least one sensory indication, and instructions to be delivered to said subject by said NF device;
a user interface;
a control circuitry, wherein said control circuitry is configured to:
deliver said at least one sensory indication to said subject using said user interface, instruct said subject to modulate said delivered at least one sensory indication by performing at least one activity;
receive electrical signals from one or more electrodes coupled to a body of said subject during the delivery of the at least one sensory indication,
measure using said electrical signals indications regarding an activity or changes thereof of at least one specific brain region or at least one specific brain network, and
provide a feedback signal to said subject indicating activity or changes thereof of said at least one brain region or said at least one brain network by continuously or intermittently modifying said at least one sensory indication, according to said measured indications.

Example 39. A system according to example 38, wherein said control circuitry is configured to measure electroencephalogram (EEG) signals based on said received electrical signals, wherein said EEG signals indicate said activity or changes thereof of said at least one specific brain region or at least one specific brain network.

Example 40. A system according to example 39, wherein said at least one specific brain region comprises at least one subcortical brain region, wherein optionally said at least one subcortical brain region comprises an amygdala, a ventral striatum, a subcortical brain region of a limbic system and/or a subcortical brain region of a reward system.

Example 41. A system according to any one of examples 38 to 40, wherein said memory of said personal device and/or said memory of said NF device store information about a mental state of said subject, and wherein said control circuitry of said personal device initiates said at least one maintenance session or determines a timing schedule for initiating said at least one maintenance session based on said mental state information.

Example 42. A system according to example 41, wherein said control circuitry of said personal device is configured to modify said at least one NF maintenance session according to said information about said mental state of said subject.

Example 43. A system according to any one of examples 41 or 42, wherein said information comprises information that said subject is diagnosed with a mental or a brain disorder, or presents a symptom of a mental or a brain disorder.

Example 44. A system according to example 43, wherein said mental or brain disorder comprises at least one of, a stress disorder, post-traumatic stress disorder (PTSD), depression, Attention deficit hyperactivity disorder (ADHD), substance use disorder (SUD), dementia, Alzheimer's disease, Mild Cognitive Impairment, Autism.

Example 45. A system according to any one of examples 41 to 44, comprising a remote device in communication with said NF device and said personal device, wherein said remote device comprises:
a memory with subject-related information comprising said information about a mental state of said subject and/or information about daily activities of said subject;
a communication circuitry configured to communicate with said NF device and said personal device;
a control circuitry, wherein said control circuitry is configured to determine a schedule of at least one additional maintenance session and/or of at least one additional NF treatment session according to said stored mental state information, and to signal said communication circuitry to deliver said schedule to said NF device and/or to said personal device.

Example 46. A system according to example 45, wherein determining said schedule by said control circuitry of said remote device comprises preceding said at least one additional maintenance session and/or said at least one additional NF treatment session if said subject-related information indicates that said subject is expected to have difficulties in said daily activities.

Example 47. A system according to any one of examples 45 or 46, wherein determining said schedule by said control circuitry comprises delaying said at least one additional maintenance session and/or said at least one additional NF treatment session if said subject-related information indicates that a mental state of said subject deteriorates.

Example 48. A system according to any one of examples 35 to 47, wherein said instructions delivered to said subject by said personal device comprise instructions to apply at least one cognitive activity, shown to modulate said delivered at least one sensory indication when applied by the subject during the at least one NF treatment session.

Example 49. A system, comprising:
a neurofeedback (NF) device configured to deliver a NF treatment to a subject for teaching said subject to modulate an activity of at least one specific brain region or at least one specific brain network, comprising:
a memory storing at least one NF protocol, a progress indication indicating a progress in an ability of said subject to modulate an activity of said at least one specific brain region or said at least one specific brain network, and a mental state indication with information about a mental state of the subject during the NF treatment;
a supervisor interface in communication with said NF device, configured to generate and display at least one visual interface, wherein said NF device is configured to signal said supervisor interface to generate and deliver said at least one visual interface with said progress indication and said mental state indication.

Example 50. A system according to example 49, wherein said at least one protocol stored in said memory comprises at least one sensory indication and instructions to be delivered to said subject by said NF device, wherein said NF device further comprises a user interface and a control circuitry, wherein said control circuitry is configured to:
deliver said at least one sensory indication to said subject using said user interface; instruct said subject to modulate said delivered at least one sensory indication by performing at least one activity;
receive electrical signals from one or more electrodes coupled to a head of said subject;
measure using said electrical signals indications regarding an activity or changes thereof of said at least one specific brain region or said at least one specific brain network, and
provide a feedback signal to said subject during said delivery of said at least one sensory indication, according to said measured indications;
wherein said control circuitry is further configured to generate based on said received electrical signals said progress indication and to generate said mental state indication based on received information about said mental state of said subject.

Example 51. A system according to example 50, wherein said control circuitry is configured to receive input signals with said information about said mental state of said subject, wherein said input signals comprise results of at least one assessment questionnaire, self-reported information, and/or input from an expert or a supervisor about said state of the subject.

Example 52. A system according to example 51, wherein said at least one assessment questionnaire comprises at least one of, a Clinician-Administered PTSD Scale for DSM-5 (CAPS-5) and/or parameters or domains of the CAPS-5 or derivatives thereof, a PTSD Checklist for DSM-5(PCL-5) or derivatives thereof, a Patient Health Questionnaire-9 (PHQ-9) or derivatives thereof, a Hamilton Depression Rating Scale or derivatives thereof, a Snaith-Hamilton Pleasure Scale (SHAPS) or derivatives thereof, a Montgomery-Asberg Depression Rating Scale (MADRS) or derivatives thereof, a Patient Health Questionnaire (PHQ) or derivatives thereof, a Clinical Global Impression (CGI) or derivatives thereof, an Adult Attention deficit hyperactivity disorder (ADHD) ADHD Investigator Symptom Rating Scale (AISRS) or derivatives thereof, an Adult ADHD Self-Report Scale (ASRS) or derivatives thereof, a Test of Variables of Attention (TOVA) or derivatives thereof, a Patient Health Questionnaire (PHQ) or derivatives thereof, an Alzheimer's Disease Assessment Scale-Cognitive Subscale (ADAS-Cog) or derivatives thereof, a mini-mental state examination (MMSE) or Folstein test or derivatives thereof.

Example 53. A system according to any one of examples 50 to 52, wherein said control circuitry is configured to determine a correlation between said progress indication and said subject mental state indication, and to signal said supervisor interface to display said determined correlation.

Example 54. A system according to any one of examples 50 to 53, wherein said control circuitry is configured to generate a trend of a change in said progress indication, and a trend of a change in said subject mental state indication over a selected time period of said NF treatment and/or predictions thereof, and to signal said supervisor interface to display said generated trend of said change in said progress indication and said trend of said change in said subject state indication, and/or of said predictions thereof.

Example 55. A system according to any one of examples 49 to 54, wherein said supervisor interface is configured to display said progress indication and said mental state indication side by side in said at least one visual interface.

Example 56. A system, comprising:
a neurofeedback (NF) device configured to deliver a NF treatment to a subject for teaching said subject to modulate an activity of at least one specific brain region or at least one specific brain network, comprising:
a memory storing at least one NF protocol, a mental state indication with information about a mental state of the subject during the NF treatment;
a supervisor interface in communication with said NF device, configured to generate and deliver at least one human detectable indication, wherein said NF device is configured to signal said supervisor interface to generate and deliver said at least one human detectable indication with said mental state indication.

Example 57. A system according to example 56, wherein said NF device comprises a control circuitry, wherein said control circuitry is configured to receive input signals with said information about said mental state of said subject and to generate said mental state indication based on said received input signals, wherein said input signals comprise results of at least one assessment questionnaire, self-reported information, and/or input from an expert or a supervisor about said mental state of the subject.

Example 58. A system according to example 57, wherein said at least one assessment questionnaire comprises at least one of, a Clinician-Administered PTSD Scale for DSM-5 (CAPS-5) and/or parameters or domains of the CAPS-5 or derivatives thereof, a PTSD Checklist for DSM-5(PCL-5) or derivatives thereof, a Patient Health Questionnaire-9 (PHQ-9) or derivatives thereof, a Hamilton Depression Rating Scale or derivatives thereof, a Snaith-Hamilton Pleasure Scale (SHAPS) or derivatives thereof, a Montgomery-Asberg Depression Rating Scale (MADRS) or derivatives thereof, a Patient Health Questionnaire (PHQ) or derivatives thereof, a Clinical Global Impression (CGI) or derivatives thereof, an Adult Attention deficit hyperactivity disorder (ADHD) ADHD Investigator Symptom Rating Scale (AISRS) or derivatives thereof, an Adult ADHD Self-Report Scale (ASRS) or derivatives thereof, a Test of Variables of Attention (TOVA) or derivatives thereof, a Patient Health Questionnaire (PHQ) or derivatives thereof, an Alzheimer's Disease Assessment Scale-Cognitive Subscale (ADAS-Cog) or derivatives thereof, a mini-mental state examination (MMSE) or Folstein test or derivatives thereof.

Example 59. A system according to any one of examples 56 to 58, wherein said human detectable indication regarding said subject mental state comprises at least one suggestion to reschedule at least one additional NF treatment session, to stop said NF treatment and/or to modify at least one parameter of said NF treatment protocol.

Example 60. A system according to any one of examples 56 to 59, wherein said human detectable indication regarding said subject mental state comprises at least one suggestion to combine said NF treatment with at least one additional treatment or to modify at least one additional treatment.

Example 61. A system according to example 60, wherein said at least one additional treatment comprises at least one of, a drug treatment, a psychological treatment and/or a behavioral treatment.

Example 62. A biofeedback device comprising:
a user interface configured to generate a human detectable indication;
a memory, wherein said memory stores a predetermined absolute range of signal amplitude values or a threshold of a signal amplitude value;
a control circuitry, wherein said control circuitry is configured to:
receive at least one first electrical signal recorded by at least one first electrode and at least one second electrical signal recorded by at least one second electrode;
process said at least one first electrical signal and said at least one second electrical signal;
identify following said process that said at least one first electrical signal and said at least one second electrical signal are within said stored predetermined absolute range of peak to peak values;
set a relative threshold of peak to peak values or a relative minimal threshold peak to peak value, based on a minimal peak to peak value in said at least one first processed electrical signal;
determine that said at least one second electrical signal has a desired signal quality if a peak to peak average value of said at least one second electrical signal is within said relative threshold of peak to peak values or above said relative minimal threshold peak to peak value;
signal said user interface to generate and deliver a human detectable indication with information whether or not said at least one second electrical signal has said desired signal quality based on results of said determine.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as detect activity or changes thereof of at least one brain region, for example a deeply located brain region, and optionally sub-cortical brain region and/or a brain network, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart of a process for monitoring a state of a subject undergoing NF treatment during the treatment and/or following the treatment, according to some exemplary embodiments of the invention;

FIG. 2 is a schematic illustrations showing interactions between components of a NF system, according to some exemplary embodiments of the invention;

FIG. 3A is a block diagram of a NF device, according to some exemplary embodiments of the invention;

FIG. 3B is a schematic illustration showing interactions between a subject undergoing a NF treatment and a personal device, according to some exemplary embodiments of the invention;

FIG. 3C is a flow chart describing activities performed by a subject using the system and methods described herein during a NF treatment delivered to the subject, according to some exemplary embodiments of the invention;

FIG. 3D is a flow chart describing activities of a subject after ending the NF treatment or at least one session of the treatment, according to some exemplary embodiments of the invention;

FIG. 3E is a flow chart describing activities performed by a system for delivery of a NF treatment, according to some exemplary embodiments of the invention;

FIG. 4A is a general scheme of a NF treatment, according to some exemplary embodiments of the invention;

FIG. 4B is a flow chart of a general NF treatment process, according to some exemplary embodiments of the invention;

FIG. 4C is a flow chart of a process for performing a NF treatment session, according to some exemplary embodiments of the invention;

FIG. 4D is a flow chart of a process for performing a NF training cycle, according to some exemplary embodiments of the invention;

FIG. 5 is a flow chart of a process for performing NF maintenance sessions, according to some exemplary embodiments of the invention;

FIG. 6A is a schematic illustration showing generation of an EFP signal or an electroencephalogram (EEG) signal indicating activity of at least one brain region and/or at least one brain network, according to some exemplary embodiments of the invention;

FIG. 6B is a schematic illustration of an active NF treatment cycle, according to some exemplary embodiments of the invention;

FIG. 7A is a general flow chart of a process for determining signal quality, according to some exemplary embodiments of the invention;

FIG. 7B is a flow chart of a process for determining signal quality, according to some exemplary embodiments of the invention;

FIG. 7C is a schematic illustration of a user interface for displaying signal quality, according to some exemplary embodiments of the invention;

FIG. 7D is a general flow chart of an additional process for determining signal quality, according to some exemplary embodiments of the invention;

FIG. 7E is a flow chart of an additional process for determining signal quality, according to some exemplary embodiments of the invention;

FIG. 7F is a schematic illustration showing signals recorded by 3 electrodes, according to some exemplary embodiments of the invention;

FIGS. 8A-8G are schematic illustrations of NF supervisor interfaces displayed by the NF system to an expert, for example a supervisor of the NF treatment, according to some exemplary embodiments of the invention;

FIG. 9A is a flow chart of a process for determining a relation between progress of a subject in a NF treatment and a state of the subject, according to some exemplary embodiments of the invention;

FIG. 9B is a schematic illustrations of NF supervisor interface showing a graphical representation of a relation between treatment progress scores and a score indicating a state of a subject during a NF treatment, according to some exemplary embodiments of the invention; and

FIG. 9C is a schematic illustration of a visual interface showing a representation of a treatment progress score during NF treatment and scores of mental state assessment questionnaires measured during the NF treatment, according to some exemplary embodiments of the invention.

### DESCRIPTION OF SPECIFIC EMBODIMENTS OF THE INVENTION

The present invention, in some embodiments thereof, relates to treatment of mental disorders and, more particularly, but not exclusively, to treatment of mental disorders using neurofeedback.

An aspect of some embodiments relates to delivering a biofeedback process, for example neurofeedback (NF) treatment to a subject while monitoring the subject state during and/or following the NF treatment and modifying the NF treatment accordingly. In some embodiments, the NF treatment is modified based on information received regarding the subject state following the treatment, for example between two consecutive treatment sessions or after a final planned treatment session. In some embodiments, at least one new NF treatment session is scheduled based on a subject state following the NF treatment. Alternatively or additionally, at least one new treatment is initiated or cancelled based on a subject state following the NF treatment.

According to some embodiments, information regarding the subject state is received using one or more questionnaires, filled by the subject himself. Alternatively, the one or more questionnaires is filled by a third party person, for example a nurse, a caregiver, a physician, a therapist, a social worker, a mental health expert or any other person trained and/or qualified to fill the one or more questionnaires. In some embodiments, the one or more questionnaires comprise the Clinician-Administered PTSD Scale for DSM-5 (CAPS-5) and/or parameters or domains of the CAPS-5, a PTSD Checklist for DSM-5 (PCL-5), and a Patient Health Questionnaire-9 (PHQ-9). Alternatively or additionally, the questionnaires comprises the Hamilton Depression Rating Scale or any derivative, the Snaith-Hamilton Pleasure Scale (SHAPS) or any derivative, the Montgomery-Asberg Depression Rating Scale (MADRS) or derivatives, the Patient Health Questionnaire (PHQ) or derivatives, the Clinical Global Impression (CGI) or derivatives. Alternatively or additionally, the questionnaires comprises the Adult Attention deficit hyperactivity disorder (ADHD) ADHD Investigator Symptom Rating Scale (AISRS) or derivatives, the Adult ADHD Self-Report Scale (ASRS) or derivatives, the Test of Variables of Attention (TOVA), or the Patient Health Questionnaire (PHQ).

In some embodiments, the one or more questionnaires are stored in a memory of a device, for example a device used to deliver the NF treatment and/or a mobile device, for example a cellular device or a tablet. Alternatively or additionally, the one or more questionnaires are stored in a memory of a remote device, for example a remote computer, a server device, a cloud memory storage and/or processing device. In some embodiments, after a subject fills the one or more questionnaires, the filled questionnaires, and/or the subject answers and/or a score of the questionnaires is stored in at least one of, a memory of a remote device, a memory of the mobile device, a memory of the NF device.

According to some exemplar embodiments, the one or more questionnaires are filled using a user interface of the device or mobile device, for example using a software stored in a memory of the device or the mobile device. Optionally, the one or more questionnaires are processed and/or analyzed using a control circuitry of the device, for example the control circuitry of the device is configured to calculate a total score of the one or more questionnaires, and optionally to determine a state, for example a mental state of the subject based on analysis results, for example based on the calculated score.

Alternatively or additionally, the subject mental state is determined based on information, for example signals, received using one or more sensors, for example one or more sensors of the device and/or one or more external sensors optionally communicating with the device. In some embodiments, the one or more external sensors comprise wearable sensors and/or sensors coupled to the subject body. In some embodiments, the one or more sensors and/or the one or more external sensors comprise at least one of, an optic sensor for example a camera, a heart rate sensor, a blood pressure sensor, a skin conductance sensor, an electromyography (EMG) sensor. In some embodiments, the subject mental state of the subject is determined based on signals received from at least one electrode configured to record electrical signals from a brain of the subject, for example EEG signals.

An aspect of some embodiments relates to generating a progress score during and/or at an end of a NF treatment session, indicating an ability of a subject to self-modulate activity of at least one specific brain region. In some embodiments, a subject state score, for example a mental state score is calculated during a NF treatment, and is presented in a shared interface with the progress score. As used herein, at least one specific brain region also includes in some embodiments at least one specific neural network. In some embodiments, the at least one specific brain region is a sub-cortical brain region. In some embodiments the progress score is based on EEG signals, optionally a subset of EEG signals, recorded from the subject brain indicating activity of the at least one specific brain region. In some embodiments, the progress score is based on a difference between EEG signals measured while the subject is engaged in active neurofeedback or a central tendency measured thereof, indicating activity of the at least one specific brain region during NF, and a reference indication stored in a memory. In some embodiments, the reference indication comprises baseline EEG signals or a central tendency measure thereof, measured before active NF, indicating a baseline activity level of the at least one specific brain region. In some embodiments, the progress score is calculated by an average EFP difference between the NF cycle and the local baseline median EFP, standardized by local baseline inter-quantile-range or other statistical grouping.

According to some embodiments, a system delivers the progress score or indications thereof to a supervisor of the NF treatment and/or to a subject treated by the NF, for example a trainee. In some embodiments, the system presents a correlation, for example a graphic correlation, between the calculated progress score and a calculated state score indicating a state, for example a mental and/or a cognitive and/or a functional state of the subject. In some embodiments, the state score is calculated based on a score of specific questionnaires in a memory of the system and/or based on a score given by an expert, for example a physician, a health care professional (HCP) and/or a supervisor of the NF treatment, to the subject and/or based on measurements of at least one physiological parameter performed on the subject. In some embodiments, the score is determined by comparing the questionnaire score of the patient or a derivative of that score, to descriptive statistics of at least one cohort of patients the patient can be associated with. It may be that the patient is associated with multiple such cohorts, in which case multiple comparisons may be performed and/or presented. Same type of display could be done for EFP and modulation performance scores.

According to some embodiments, the system determined a correlation between the progress score and the mental state score and generates and optionally delivers an indication with a correlation score. In some embodiments, the system delivers the correlation score to the supervisor and/or to the subject.

An aspect of some embodiments relates to maintaining an effect of a NF treatment delivered to a subject suffering from a mental disorder, when the subject is outside the clinic and is optionally engaged in daily activities. In some embodiments, the maintenance is achieved by delivering an indication, for example a sensory indication or a trigger, for example a sensory trigger to the subject which evokes at least part of a NF treatment session delivered to the subject. Optionally, the sensory indication comprises at least one trigger selected to evoke at least part of the NF treatment session delivered to the subject. In some embodiments, an effect of the NF treatment comprises an ability of a subject trained with the NF treatment to perform a specific activity, for example, to apply a strategy, for example a mental or a cognitive strategy, in order to control an activity of at least one specific brain region or a specific brain network. Optionally, the specific brain network comprises the specific brain region.

According to some embodiments, the trigger delivered during a maintenance session is selected to evoke at least part of a NF session delivered to the subject, for example in a medical or a therapy center. In some embodiments, the trigger is selected and/or is configured to evoke at least part of the NF session, for example, a sensory indication, a feedback signal, instructions, a specific activity, for example a mental or a cognitive strategy, used during the NF session. In some embodiments, the trigger or at least a portion of the trigger is at least partially similar, to the at least part of the NF treatment session, for example to the sensory indication, to the feedback signal, to the instructions, to the specific activity, for example to the mental or to the cognitive strategy, used during the NF session. In some embodiments, the similarity between the trigger and the at least part of the NF treatment session is similarity in one or more visual and/or audio parameters, shared between the trigger and the at least part of the NF treatment session.

In some embodiments, the degree of similarity between the at least one trigger, used during a maintenance session and the at least part of the NF treatment session is at least 1%, for example at least 10%, at least 20%, at least 40%, at least 50%, at least 70%, at least 80% or any intermediate, smaller or larger percentage value. In some embodiments, the trigger is identical to the at least part of the NF treatment session, for example the trigger is the same sensory indication, feedback signal, visual interface, audio interface and/or instructions used in NF treatment session.

According to some embodiments, the effect is maintained by presenting to the subject a sensory indication which optionally includes a scenario that was presented during at least one NF treatment session in the clinic, and optionally instructing the subject to apply a mental strategy that was previously used in the at least one NF treatment session. In some embodiments, the subject is instructed to try and attenuate a mental and/or an emotional reaction to the scenario by applying the mental strategy. Optionally, the presented scenario is a new scenario that was not presented to the subject in the treatment session. Additionally or optionally, the new scenario is a variation of the scenario presented to the subject during the NF treatment session.

According to some embodiments, the maintaining is performed during one or more maintaining sessions. In some embodiments, the one or more maintaining sessions are performed according to a predetermined schedule. In some embodiments, the predetermined schedule is modified based on a mental state of the subject. Alternatively or additionally, the one or more maintaining sessions are scheduled or performed when determining that a mental state of the subject is not a target, for example a desired, mental state, for example when determining that the mental state of a subject is lower or higher than a predetermined value. Alternatively or additionally, at least one maintenance session is scheduled or is performed, based on a predicted mental state of the subject. According to some embodiments, at least one maintenance session is performed or scheduled based on input received from the subject. Optionally, at least one additional NF treatment session is performed or scheduled based on input received from the subject or from at least one of, an expert, a supervisor of the NF treatment and/or a caregiver. Alternatively or additionally, the input is received from at least one device or sensor measuring at least one physiological parameter, for example heart rate, EFP signal, blood pressure, skin conductance, pupil dilation. In some embodiments, the input comprises information or an indication that said subject is currently experiencing or expects to have difficulties, for example mental difficulties, cognitive difficulties and/or behavioral difficulties. In some embodiments, the input comprises results of one or more assessment exams, for example one or more questionnaires.

According to some exemplary embodiments, the system for delivery of the NF treatment performs assessment of the subject state during the NF treatment, before, during and after the subject is actively engaged in NF sessions. In some embodiments, the assessment is based on input received from the subject or an expert. Alternatively or additionally, the assessment is based on input generated or measured by the system, for example base on a change in an EFP signal or any other indication signal generated by the system indication an activity or a change in activity of at least one specific brain region or brain network. In some embodiments, the system modifies, or deliver suggestions to modify, the NF treatment delivered to the subject based on the results of the subject state assessment. Alternatively, the system delivers suggestions to modify an overall treatment delivered to the subject, for example adding, modifying or stopping a psychotherapy or a pharmacotherapy treatment.

According to some embodiments, an assessment of a subject state comprises an assessment of a current subject state relative to a desired, for example a target state or a reference state. Alternatively or additionally, an assessment of a subject state comprises an assessment of a deterioration or improvement in the subject state. In some embodiments, identifying a deterioration or improvement in a subject state or any other indication regarding the current state of the subject is used as an input to the system for determining whether to change or modify a treatment given to the subject, and/or for determining if to conduct additional tests.

An aspect of some embodiments relates to determining a quality of a signal measured by an electrode coupled to a body. In some embodiments, an indication is generated regarding a quality of the signal. Optionally, the indication is a human detectable indication, which optionally indicates or includes instructions to improve apposition and/or contact of the electrode with the body in case that the determined signal quality is lower than a desired, for example a target quality level. In some embodiments, the signal quality is determined prior to initiating a biofeedback process, for example biofeedback treatment or training process when one or more electrodes are coupled to the subject body, and/or during the biofeedback process, for example prior to and/or during a neurofeedback process.

According to some exemplary embodiments, the quality of the signal is determined by comparing a parameter of the recorded signal, for example peak to peak value, optionally within a selected epoch, to a predetermined absolute value threshold, which is optionally a predetermined range of values, and then optionally to a relative threshold. In some embodiments, the relative threshold is determined with respect to a peak to peak value of a selected electrode. In some embodiments, the selected electrode is an electrode which records a signal with the minimal peak to peak value. Optionally, in case that the relative threshold is too close to a minimal value of the absolute threshold, a second absolute threshold is used, which defines a narrower subrange within the absolute value range or a larger lower limit of the absolute threshold.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Exemplary general process for monitoring patient state According to some exemplary embodiments, a subject diagnosed with a mental disorder and/or that suffers from one or more symptoms of a mental disorder is treated using a NF treatment, for example a NF training. In some embodiments, the NF treatment is used to train the subject to control activity, for example upregulate or downregulate activity of at least one brain region or at least one brain network. In some embodiments, the brain region and/or the brain network are related to the mental disorder. Alternatively or additionally, the brain region and/or the brain network are related to the one or more symptoms. In some embodiments, modifying an activity of the brain region or the brain network in a desired direction, for example upregulate or downregulate), attenuates the appearance of the one or more symptoms.

According to some exemplary embodiments, the NF treatment, for example the NF training, teaches the subject to control the activity of the at least one brain region or the at least one brain network or biomarker associated with these regions or networks, by instructing the subject to apply a specific strategy, for example a task or a cognitive task, and providing feedback to the subject regarding the activity of the at least one brain region and/or the at least one brain network while applying the strategy. In some embodiments, the provided feedback allows the subject to personalize a strategy or to select a specific strategy out of two or more strategies that is better in directing the activity of the brain region or the brain network in the desired direction, when applied.

According to some exemplary embodiments, during the NF treatment, the subject state is determined either by receiving subjective input or objective input. In some embodiments, the subject state is determined during the NF treatment, for example to allow personalization of the NF treatment to the subject, optionally by modifying the NF treatment according to the subject state prior to a NF treatment session and/or during a NF treatment session. Optionally, an overall treatment plan which optionally includes other treatments other the NF treatment is modified according to the subject state.

According to some exemplary embodiments, a system for providing the NF treatment, provides the treatment at a treatment center and outside a treatment center, for example at a home of a subject receiving the treatment, for example a patient or a trainee. In some embodiments, the system monitors the reaction of the subject to the treatment during the treatment. Alternatively or additionally, the system monitors the subject state, for example mental and/or cognitive and/or functional state, between treatment sessions. Alternatively or additionally, the system is responsive to input from the subject, for example when the subject is not feeling well, or when the subject is expected not to feel well in the future optionally due to exposure to a trigger of a mental condition and/or due to a planned situation.

According to some exemplary embodiments, the system provides the NF treatment, for example treatment sessions that involve providing a feedback signal to the patient or trainee, in a treatment center and outside the treatment center. Optionally, the system provides the NF treatment, for example treatment sessions that involve providing a feedback signal to the patient or trainee, outside the treatment center, where optionally the patient is invited one or more times to meet an expert in the treatment center.

According to some exemplary embodiments, the system automatically modifies or suggest to modify the NF treatment provided to the subject, at the treatment center and/or at home in response to at least one of, monitored subject state during NF treatment, monitored subject state between treatment sessions, and/or input received from the subject. In some embodiments, the system suggest to modify, stop or initiate at least one additional treatment, for example a drug, a psychological or a behavior therapy, in response to at least one of, monitored subject state during NF treatment, monitored subject state between treatment sessions, and/or input received from the subject. Alternatively or additionally, the system invites the subject to a meeting with an expert, for example a physician or a therapist, in response to at least one of, monitored subject state during NF treatment, monitored subject state between treatment sessions, and/or input received from the subject.

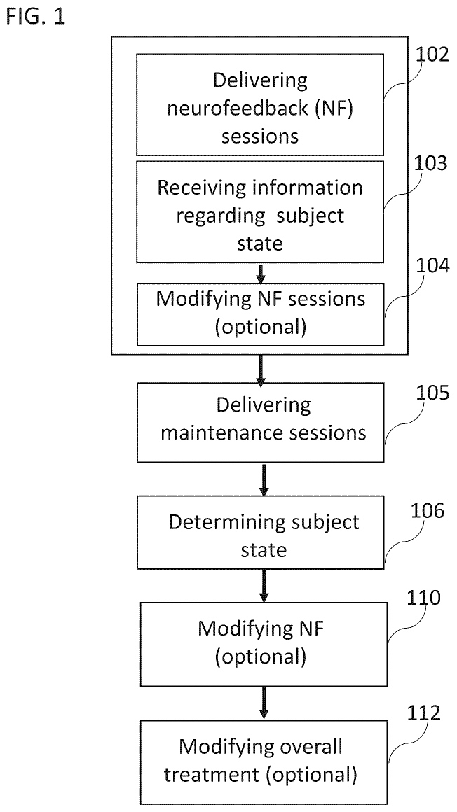

Reference is now made to FIG. 1, depicting a general process for monitoring a state of a subject undergoing a NF treatment during the treatment and/or following the treatment, according to some exemplary embodiments of the invention;

According to some exemplary embodiments, a NF treatment is delivered at block 102. In some embodiments, the NF treatment is delivered in one or more, for example two or more treatment sessions. In some embodiments, the NF treatment sessions are performed in a treatment center, for example an office, or a clinic. Alternatively, the NF treatment sessions are performed at home, for example using a device that measured EEG signals. In some embodiments, during a NF treatment session, a scenario is presented to the subject. In some embodiments, the scenario is specific to the subject. Alternatively, the scenario is non-specific. In some embodiments, the presented scenario is selected according to the mental disorder of the subject and/or according to the one or more symptoms.

According to some exemplary embodiments, a feedback signal indicating an activity of at least one brain region or brain network or biomarker thereof, or changes thereof is delivered to the subject while presenting the scenario to the subject. Optionally, the scenario, for example at least one parameter of the scenario is modified according to the indicated activity or changes thereof. In some embodiments, the subject is instructed to apply at least one mental strategy, for example a cognitive task, to try and modulate the activity of the at least one brain region or brain network or biomarker thereof. In some embodiments, modifying the activity of the at least one brain region or the at least one brain network, modifies the presented scenario, for example to provide covert feedback to the subject regarding his success in modifying the activity of the brain region or brain network in a desired direction. In some embodiments, the neurofeedback is delivered as described in international patent applications publication numbers WO2020121299A1, WO2020100144A1, and WO2021260697A1, incorporated herein as a reference in their entirety.

According to some exemplary embodiments, during a treatment session, information regarding a state of the subject is received, at block 103. In some embodiments, the information is received at a beginning of a treatment session, in between neurofeedback blocks in each session and/or at the end of the treatment session. In some embodiments, the information is received using one or more questionnaires, filled by either the subject, for example a trainee of the NF training, or by a therapist, for example a nurse, a caregiver or an expert who is optionally qualified to fill the questionnaires. Alternatively or additionally, the information is received using one or more sensors, configured to sense at least one physiological parameter of the subject, for example heart rate, blood pressure, skin conductance, and/or oxygen saturation. Optionally, the information is received using a camera.

According to some exemplary embodiments, the NF treatment sessions are optionally modified, at block 104. In some embodiments, at least one parameter of the NF treatment is modified at block 104 based on the information received at block 103. In some embodiments, the at least one parameter comprises at least one of, timing of NF treatment sessions, duration of one or more treatment sessions, duration of an interval between treatment sessions, and interface displayed to the subject.

In some embodiments, the NF sessions are modified based on information received during at least one NF session and/or based on information received between treatment sessions.

According to some exemplary embodiments, maintenance sessions are delivered ay block 105. In some embodiments, maintenance sessions are delivered to the subject without providing a feedback signal to the subject. In some embodiments, the maintenance sessions are delivered when the subject is outside the medical center, for example when the subject is at home and/or at work.

According to some exemplary embodiments, a state of the subject is determined at block 106. In some embodiments, the state, for example mental state, of the subject is determined at block 106 based on the information received at block 104. In some embodiments, the mental state of the subject is determined at block 106 during a training session. Alternatively or additionally, the mental state of the subject is determined between treatment sessions, for example based on information received when the subject is outside the treatment center, for example when the subject is at home and/or is involved in daily activities.

According to some exemplary embodiments, the NF treatment is optionally modified at block 110. In some embodiments, the NF treatment is optionally modified at block 110 based on the determined mental state of the subject. In some embodiments, if a determined mental state of the patient during a NF treatment session indicates an increase in severity of at least one symptom of the mental state and/or a deterioration in the mental state, the NF treatment session is stopped or shortened. Alternatively or additionally, a sequence of NF treatment sessions is altered, for example timing of one or more blocks or portions of a treatment session is changed, and/or an order of treatment session blocks or portions is modified. Alternatively or additionally, at least one parameter of the NF treatment session is modified, for example duration of each NF block, interval between two consecutive NF blocks, type of scenario presented to the subject in each NF block, response rate of interface, for example visual and/or audio interface, type of interface, complexity of interface, color of interface or other natural behavior of the interface, and/or type of strategy to be used by the subject. For example, if a determined mental state of the patient during a NF treatment session indicates an increase in severity of at least one symptom of the mental state and/or a deterioration in the mental state, the subject is instructed to at least one of, apply a different mental strategy, modify an existing mental strategy, or select a different mental strategy out of a group of mental strategies offered to the subject.

According to some exemplary embodiments, if a determined mental state of the patient between NF treatment sessions indicates an increase in severity of at least one symptom of the mental state and/or a deterioration in the mental state, the subject is instructed to at least one of, set a meeting or arrive to a meeting with a therapist, set a NF treatment session or arrive at a NF treatment session, and/or initiate a maintenance NF session outside the treatment center.

According to some exemplary embodiments, if a determined mental state of the patient between during NF treatment sessions indicates a reduction in severity of at least one symptom of the mental state and/or an improvement in the mental state, the NF treatment session is shortened. Alternatively, the NF treatment session proceeds as planned.

According to some exemplary embodiments, an overall treatment delivered to the subject is optionally modified based on the subject state determined during the NF treatment session and/or between treatment sessions, at block 112.

According to some exemplary embodiments, if a determined mental state of the patient between or during NF treatment sessions indicates an increase in severity of at least one symptom of the mental state and/or a deterioration in the mental state, the subject is instructed to at least one of, set a meeting or arrive at a meeting with a therapist, and/or modify a regime of at least one drug, for example increase a dosage of the at least one drug.

According to some exemplary embodiments, if a determined mental state of the patient between or during NF treatment sessions indicates a reduction in severity of at least one symptom of the mental state and/or an improvement in the mental state, the subject is instructed to reduce a dosage of at least one drug.

### Exemplary NF System

According to some exemplary embodiments, the NF system is configured to be used for treating, for example training subjects diagnosed with a mental disorder according to the Diagnostic and Statistical Manual of Mental Disorders (DSM) and/or subjects that suffer from one or more symptoms of a mental disorder. In some embodiments, the NF system is configured to deliver an overall therapeutic environment for the subjects that optionally provides treatment, monitoring, data driven-decision assistance and guidance for patients an therapists. In some embodiments, the system is configured to provide a personalized and remote treatment and monitoring.

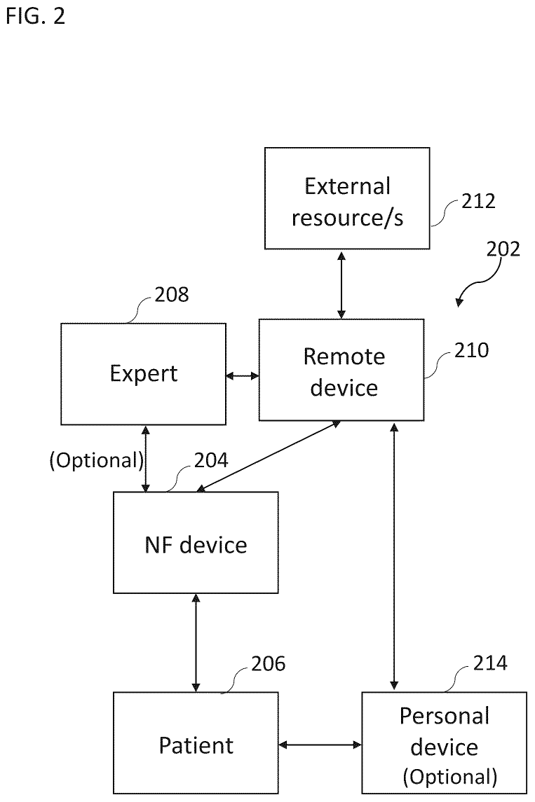

Reference is now made to FIG. 2, depicting interactions between components of a NF treatment system, according to some exemplary embodiments of the invention.

According to some exemplary embodiments, a NF system, for example system 202 comprises a NF device 204, configured to deliver a NF treatment to a subject, for example patient 206. In some embodiments, the NF device 204 is stationary, and is located at a treatment center. Alternatively, the NF device 204 is portable. Optionally the NF device 204 comprises a pod which is size and shaped to allow the subject to receive the NF treatment while being located inside the pod. In some embodiments, the pod is configured to generate an isolated environment, for example an acoustic isolated environment. In some embodiments, the pod comprises a door that is configured to be closed when the subject is within the pod, for example during the NF treatment session.

According to some exemplary embodiments, the NF device 204 comprises at least one user interface for interacting with the patient 206 during the NF treatment session. In some embodiments, the user interface presents the patient information regarding the NF training, and/or during the NF training, for example presents at least one scenario or a stimulus to the patient. Alternatively or additionally, the patient interface delivers instructions to the patient 206, before, during and/or after the NF training. In some embodiments, the user interface receives input from the patient 206, for example the patient 206 stops the NF training and/or request help during the NF training. According to some exemplary embodiments, the NF device 204 optionally comprises at least one expert interface, for example a supervisor interface, for interacting with an expert 208. In some embodiments, interacting, for example communicating with the Expert 208 comprises providing information on the patient 206 before, during and/or after a NF treatment session. Alternatively or additionally, interacting with the Expert 208 comprises providing information about the progress of the NF treatment sessions and/or about the performance of the patient 206 during the NF treatment session. In some embodiments, the expert interface receives input from the expert 208. In some embodiments, the expert 208 modifies one or more parameters of the NF treatment, or stops the NF treatment using the expert interface.

According to some exemplary embodiments, the system 202 comprises a remote device 210, for example a server, a remote computer, a cloud for example a cloud data storage and/or a cloud data processing. In some embodiments, the remote device is optionally located outside the treatment center in which the NF device is positioned, for example at home. In some embodiments, the remote device 210 is in communication with the NF device via wireless and/or wired communication. Optionally, the remote device 210 is in communication with a plurality of NF devices, each is located at a different treatment center. In some embodiments, the remote device 210 generates and/or stores one or more databases in a memory of the remote device. In some embodiments, the databases are generated based on the information received from the expert and/or from the patient 206. In some embodiments, the remote device stores at least one of, a formula, an algorithm and/or a lookup table, for example, for processing information received from the patient and/or information received from the expert 208.

According to some exemplary embodiments, the remote device 210 stores one or more NF treatment protocols, and/or one or more overall treatment procedures, for example psychological procedures and/or drug-related procedures, for treating one or more mental disorders. Optionally, an expert, for example a health care provider inserts a protocol into a memory of the device, for example a basic protocol that can be modified during the NF treatment. Alternatively, the remote device stores a basic protocol or a model for a protocol that is optionally modified to at least one active protocol based on information received from the user and/or a NF device and/or an expert for example a health care provider. Optionally, the information is collected when performing at least part of a NF treatment. In some embodiments, the remote device 210 uses the one or more formula, algorithm and/or a lookup table, to generate new NF treatment protocols, for example NF treatment protocols personalized for a specific patient or a group of patients, based on information stored in the remote device 210. Alternatively, the remote device 210 uses the one or more formula, algorithm and/or a lookup table, to modify an existing NF treatment protocol, for example to personalize a NF treatment protocol for a specific patient or a group of patients, based on information stored in the remote device 210.

According to some exemplary embodiments, the remote device 210 uses the one or more formula, algorithm and/or a lookup table, to generate a new overall treatment plan, for example a treatment plan personalized to a specific patient or group of patients that includes a NF treatment protocol and at least one additional treatment, based on information stored in the remote device 210. Alternatively, the remote device 210 uses the one or more formula, algorithm and/or a lookup table, to modify an existing treatment plan, for example to personalize the existing treatment plan for a specific patient or a group of patient, based on information stored in the remote device 210. In some embodiments, the at least one additional treatment comprises at least one of, a psychological treatment, a cognitive behavioral treatment (CBT), and a drug treatment.

According to some exemplary embodiments, the remote device 210 uses the one or more formula, algorithm and/or a lookup table, to generate suggestions or recommendations for at least one NF treatment protocol and/or at least one treatment plan, for example an overall treatment plan, based on information stored in the remote device 210. In some embodiments, the remote device 210 is configured to remotely modify and/or replace an existing protocol, for example a NF protocol, stored in the NF device.

According to some exemplary embodiments, the remote device 210 is in communication with the expert 208. In some embodiments, the expert 208 receives information from the remote device 210 and/or provides information to the remote device 210. Optionally, the remote device 210 is in communication with the expert 208 via a personal device of the expert, for example a mobile device or a personal computer.

According to some exemplary embodiments, the system 202 further comprises a software program, for example an application software or a software suite, installed on a personal device 214 of the patient. In some embodiments, the personal device 214 comprises a computer, a cellular device, a mobile device, a tablet, and/or a wearable device. In some embodiments, the personal device 214 is configured to receive information from the patient 206 and//or to deliver information to the patient 206, for example using the software program installed in a memory of the personal device 214. Optionally, the personal device 214 is configured to detect or measure at least one parameter related to a behavior of the patient 206, for example posture of the patient 206, sleeping time of the patient 206, content created, visualized, engaged and/or searched by the patient 206 using the personal device, and operating habits of the device 214. Optionally or additionally, the personal device 214 measures at least one physiological parameter of the patient 206, optionally using one or more sensors of the device and/or using one or more sensors of a devices that are in communication with the personal device 214. In some embodiments, the persona device 214 stores one or more questionnaires, for example assessment questionnaires. In some embodiments, the patient fills at least one questionnaire stored in the memory of the personal device 214, according to a planned schedule and/or on demand, for example when receiving a request from the remote device 210 and/or the expert to fill the questionnaire, or to modify the questionnaires filling schedule.

According to some exemplary embodiments, the personal device 214 measures and/or collects data related to the patient 206 behavior or state, when the patient 206 is at the treatment center and/or when the patient 206 is outside the treatment center, for example when the patient 206 is at home, at work and/or involved in daily activities. In some embodiments, the personal device collects the information from the patient using an artificial intelligence driven software application stored in the memory of the personal device 214 that is configured to generate discussions with the patient 206 on general subjects and/or on subjects related to the mental disorder, to analyze the patient 206 response during the discussions, and to determine a patient state based on the analysis results. In some embodiments, the application software analyzed the patient response by identifying words or phrases indicating a specific state, for example a mental state of the patient. In some embodiments, the application software calculates if a frequency of the words or phrases appearance is higher than a predetermined threshold, indicating a severity level of a mental state of the patient.

According to some exemplary embodiments, measurements measured or collected by the personal device 214, optionally using the software program, is transmitted, optionally anonymously, to the remote device 210. In some embodiments, the remote device 210 uses the information received from the personal device 214 and/or from the NF device 204, to determine and/or predict a state, for example a mental state, of the patient 206. In some embodiments, the remote device 210 uses the information received from the personal device 214 and/or from the NF device 204 to at least one of, determine if a NF treatment protocol fits a current or a predicted patient state, determine if an overall treatment provided to the patient fits a current or a predicted patient state, suggest modifications of an existing NF treatment protocol and/or an existing overall treatment, suggest a new NF treatment protocol and/or a new overall treatment, and generate a new NF treatment protocol and/or a new overall treatment.

According to some exemplary embodiments, the remote device 210 is configured to deliver an indication regarding the current state and/or the predicted state of the patient 206, to the expert 208 and/or to the patient via the application software installed in personal device 214, or an application software installed in a personal device of the expert 208. In some embodiments, the indication includes suggestions how to modify a NF treatment of the patient and/or how to modify an overall treatment of the patient 206. Alternatively or additionally, the indication comprises predictions of a future patient state, if proceeding with an existing NF treatment and/or an overall treatment plan. Alternatively or additionally, the indication comprises predictions of a future patient state, if the existing NF treatment and/or the existing overall treatment is modified according to delivered suggestions. Alternatively or additionally, the indication comprises predictions of a future patient state, if replacing the existing NF treatment and/or the existing overall treatment with a new treatment plan, based on the delivered suggestions.

According to some exemplary embodiments, the personal device is configured to monitor and determine a subject state optionally a mental state, by collecting and/or analyzing data related to the use of one or more applications and software programs installed in a memory of the personal device. For example, social media applications, mental health applications, and/or applications related to the NF treatment. In some embodiments, the one or more parameters comprise content and/or input inserted or posted by the subject and/or timing parameters related to the use of the applications and/or software programs.

According to some exemplary embodiments, the remote device 210 is in communication with one or more external resources 212, for example health providers, health maintenance organizations (HMO's), insurance companies, hospitals, and clinics. In some embodiments, the remote device 210 delivers information to the one or more external resources about at least one of, the progress of the patient receiving the NF treatment, the progress of the NF treatment, for example number of sessions performed and/or planned sessions, suggestion to stop, delay or initiate a NF therapy in a patient, suggestion to stop, delay or initiate an additional or a different treatment in the patient, for example a drug treatment, or any suggestion or recommendation generated by the system 202 and/or by the remote device with respect to a patient or a group of patients.

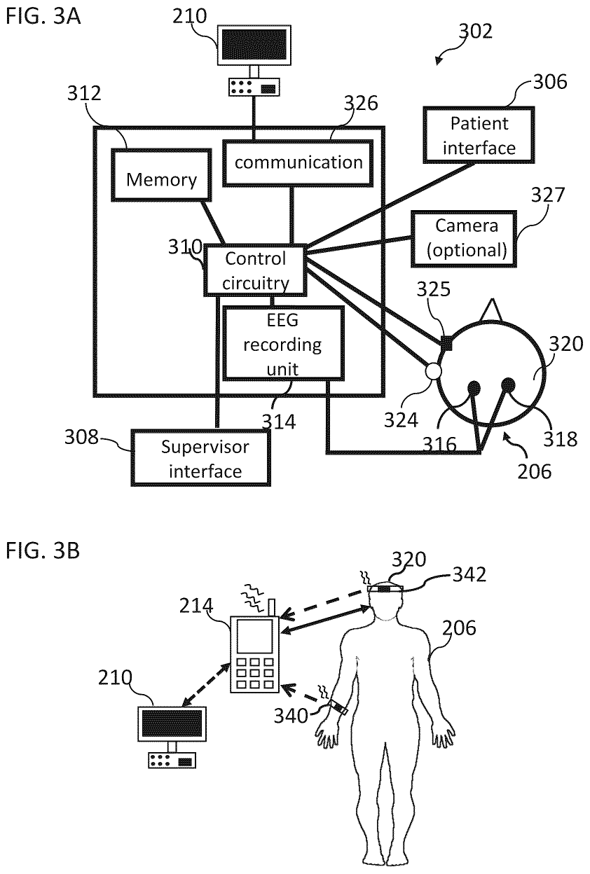

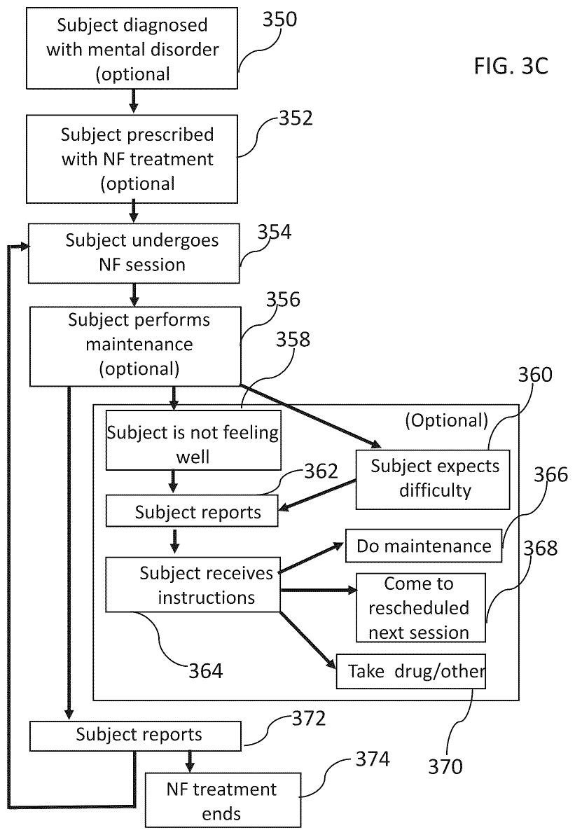

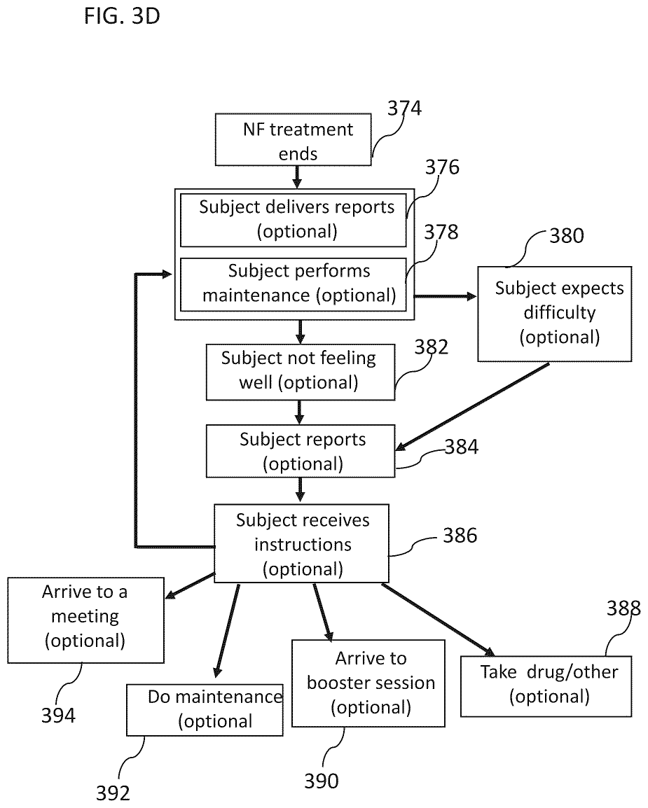

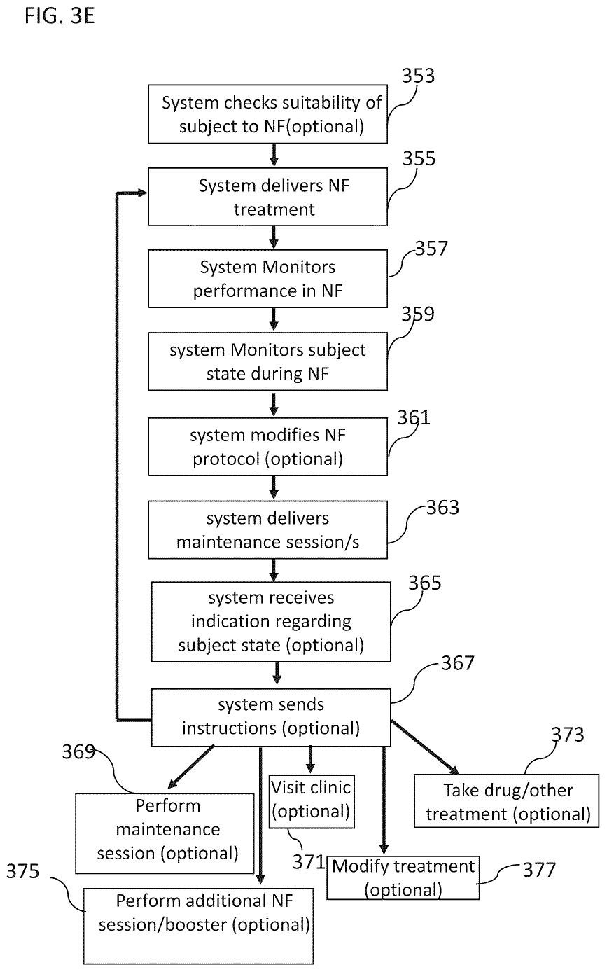

Reference is now made to FIG. 3A depicting a NF device, or a NF system according to some exemplary embodiments of the invention.

According to some exemplary embodiments, a NF system 302, for example NF device 204 shown in FIG. 2, comprises a control unit 304, a patient interface 306 and a supervisor interface 308. In some embodiments, the patient interface 306 and the supervisor interface 308 are functionally coupled to a control circuitry 310 of the control unit 304. In some embodiments, the control unit 304 further comprises a memory 312 which stores at least one algorithm and/or a lookup table, to be used when determining an activity level of at least one brain region, for example a limbic brain region, a mesolimbic brain region, a brain region of the reward system, a brain region of a positive valence system, a brain region of the negative valence system. Alternatively or additionally, the memory 618 comprises at least one EFP, for example an EFP model of the at least one mesolimbic brain region, for example the EFP model described in International Patent Application Publication number WO2021260697A1, incorporated herein as a reference in its entirety, or an EFP model of at least one limbic brain region, for example the amygdala described in International Patent Application Publication number WO2012104853A2, incorporated herein as a reference in its entirely. In other embodiments, the EFP model is stored on the EEG or on the computer. In some embodiments, the EFP model allows to correlate EEG signals recorded from a specific set of electrodes, having a specific frequency range or specific frequency ranges, during a specific time widows, with fMRI-BOLD activity of at least one specific brain region or at least one specific network.

According to some exemplary embodiments, the control unit 304 comprises an EEG recording unit 314 functionally coupled to the control circuitry 310. In some embodiments, the EEG recording unit 314 delivers signals from one or more electrodes coupled to a head of a subject, for example attached to the skull, implanted within the brain in a cortical region or implanted in a sub-cortical region of the brain, to the control circuitry 310 for processing of the received signals and analysis of the processed signals in order to determine an activity level of the at least one brain region, and/or a relation between the determined activity and at least one reference value or indication thereof, stored in the memory 312. In some embodiments, the control circuitry 310 determines the activity level or the relation between the activity level and the at least one reference value or indication thereof, using the at least one of an algorithm, a lookup table and an EFP stored in the memory 312, for example as described in WO2021260697A1 or in WO2012104853A2, incorporated herein as a reference in their entirety.

According to some exemplary embodiments, the EEG recording unit is functionally coupled to at least one electrode, for example a plurality of electrodes 316 and 318 positioned on a head 612 of a patient 206. In some embodiments, the plurality of electrodes comprise 2, 3, 4, 5, 6, 7, 8, 9, 10 or any larger number of electrodes positioned and optionally attached to the head 320 of the patient 206. Optionally, the plurality of electrodes are arranged in an array. In some embodiments, the electrodes are positioned at specific one or more locations on a head of the subject, for example at locations C3, C4, Cz, FCZ, P3, Pz and P4 of the extended 10-20 coordinate system. Alternatively, electrodes are positioned in any location or combination of locations of the extended 10-20 coordinate system. In some embodiments, the control unit 304 is connectable to at least one speaker or earphone 324 configured to deliver an audio signal to the patient 206. In some embodiments, the audio signal is delivered to the patient 206 using the patient interface 306.

According to some exemplary embodiments, the control unit comprises a communication circuitry 326 configured to receive and/or deliver signals, for example wireless signals to a remote device 210 located outside the supervisor clinic, for example to a remote computer, a remote server or a remote cloud. In some embodiments, the remote device 210 stores the at least one algorithm, lookup table and/or the EFP. In some embodiments, the control unit 304 transmits the electrical signals or processed electrical signals to the remote device 210 via the communication circuitry 326 and receives via the communication circuitry 326 signals which indicate an activity level of the brain region and/or a relation between the activity level of the brain region and a reference value indicating a target activation level of the brain region.

According to some exemplary embodiments, the control circuitry 310 signals the patient interface 306 to display on a screen a visual interface and/or to generate an audio interface and/or a tactile interface and/or an olfactory interface which continuously changes in response to an activation level or changes in the activation level relative to a baseline, of the brain region. In some embodiments, the interface is updated every 0.1, 0.5, 1, 2, 5 seconds, every 10 seconds, every 15 seconds, every 20 seconds, every 25 seconds or any intermediate, smaller or larger value, based on the signals received form the electrodes, for example electrodes 316 and 318. In some embodiments, the patient interface 306 changes the visual interface presented to the patient in a delay of about 5 seconds, about 10 seconds, about 15 seconds, about 20 seconds, about 25 seconds, or any intermediate, smaller or larger value, relative to the timing in which the electrical signal is received from the electrodes 316 and 318. In some embodiments, the patient interface comprises a display and/or speakers.

According to some exemplary embodiments, a sensory indication to the patient, for example a patient interface, is delivered in two dimension (2D), or in three dimension (3D) with or without an auditory interface. Alternatively, the interface is an auditory interface only. In some embodiments, the patient interface is delivered using virtual reality, augmented reality, with or without at least one of, audio, olfactory and tactile feedback.

According to some exemplary embodiments, the control circuitry 310 signals the supervisor interface 308 to deliver visual and/or audio indications to a supervisor of the treatment. In some embodiments, the visual and/or audio indications present at least one of, a progress of the patient during the treatment, for example during the NF cycles 240, activity level of the brain region, changes in the activity level of the brain region, difference between a reference value and an activity level of the brain region, stage of the NF session, actions of the system and indications or cues delivered to the patient. Optionally, the supervisor interface 308 is a personal device of the supervisor coupled to the control unit 304 and/or to the remote device 210, via the communication circuitry 326. In some embodiments, the control unit 304 transmits at least one of, data collected from the patient, data transmitted or displayed to the supervisor, to the remote device, for example for generating a database. In some embodiments, the dataset includes information collected from a plurality of systems and/or from a plurality of patients.

According to some exemplary embodiments, the system 302 comprises one or more sensors 325 coupled to the patient body, for example to head 320, during the NF treatment. In some embodiments, the one or more sensors 325 are configured to sense at least one physiological parameter of the patient 206, for example heart rate, blood pressure, and/or skin conductance, before, during and/or after a treatment session, while the patient is in the treatment center. In some embodiments, the system comprises at least one camera 327, configured to capture visual appearance of the patient 206 before, during and/or after a treatment session, while the patient is in the treatment center.

According to some exemplary embodiments, the control circuitry 310 and/or the remote device 210 is configured to determine a patient state, when the patient is in the treatment center, before, during and/or after a treatment session, based on at least one of, signals received from the one or more sensors 325, signals received from the camera 327, and/or base don EEG signals measured using the electrodes 316 and 318.

According to some exemplary embodiments, the signals received from the one or more sensors 316 and 318, the camera 327 and/or the electrodes 316 and 318, are used to determine if the patient 206 is in a state that is suitable to initiate active NF, or that the patient state is not suitable for initiating active NF. Alternatively or additionally, the received signals are used to determine is a patient state is suitable for continuing with active NF, while the patient receiving active NF or that the patient state indicates that the active NF should be stopped or shortened.

Reference is now made to FIG. 3B, depicting, interaction of a patient with a personal device while receiving treatment, for example a treatment for a mental disorder, according to some exemplary embodiments of the invention.

According to some exemplary embodiments, at least one application software sored in a memory of a personal device 214 is used to collect information from the patient, for example using one or more sensors coupled to the patient 206 body. In some embodiments, the one or more sensors are sensors of at least one wearable device, for example a smartwatch 340, a band, a bracelet, and/or a head band 342. In some embodiments, the wearable device is coupled, for example attached, to the patient body and is in communication, for example wireless communication with the personal device 214. In some embodiments, the one or more sensors of the wearable device comprise at least one gyroscope and/or an accelerometer, configured to sense a position, posture and/or orientation of the patient body. Alternatively or additionally, the wearable device is configured to sense at least one physiological parameter, for example hear rate, blood pressure, and/or skin conductance.

According to some exemplary embodiments, the wearable device is configured to receive signals from the one or more sensors and/or to transmit signals to the personal device 214, while the patient 206 is in a treatment center, optionally receiving NF treatment and/or when the patient is outside the treatment center, for example at home and/or engaged in everyday activities. In some embodiments, one or more sensors of the personal device 214 are used to sense the at least one physiological parameter and/or a position, posture and/or orientation of the patient body 206. In some embodiments, the personal device 214 transmits signals or indications thereof, received form the wearable device and/or from sensors of the personal device to the remote device 210. In some embodiments, the remote device 210 and/or the personal device 214 are configured to determine a patient state, based on the received signals or indications thereof. In some embodiments, the remote device 210 and/or the personal device 214 are configured to determine an engagement level of the patient 206 with a treatment, for example with the NF treatment, based on the received signals, by optionally identifying patient everyday habits, for example sleeping, physical exercising, outdoor activities, outdoor time, and social interactions habits.

According to some exemplary embodiments, at least one of control circuitry 310, memory 312, communication circuitry 326, EEG recording unit 314, supervisor interface 308, and/or patient interface 306 are components of system 302 or system 202 shown in FIG. 2.

### Exemplary Flow of Subject Activities

According to some exemplary embodiments, the system for delivery of a NF treatment, is used as a treatment for a subject, for example a human subject, diagnosed with a stress disorder. Alternatively or additionally, the system is used to treat a subject having behavioral, cognitive and/or social difficulties. In some embodiments, the system is used to manage the NF treatment and optionally additional treatments provided to the subject. Additionally, the system is used to monitor the state of the subject when the subject is between NF treatment sessions and/or is engaged in daily activities. Alternatively or additionally, the system is used to provide suggestions and/or recommendations to the subject when the subject experiences mental, social and/or behavioral difficulties, or expects to experience these difficulties in the future.

Reference is now made to FIG. 3C depicting a flow of activities performed by a subject using the system, according to some exemplary embodiments of the invention.

According to some exemplary embodiments, a subject is optionally diagnosed with a mental disorder, at block 350. In some embodiments, the subject is diagnosed with a mental disorder according to the DSM. In some embodiments, the subject is diagnosed by an expert, for example a physician, or a psychiatrist. In some embodiments, diagnosis of the subject comprises characterization of the type, stage and/or severity of the mental disorder.

According to some exemplary embodiments, the subject is optionally prescribed with a NF treatment, at block 352. In some embodiments, the subject is prescribed with the NF treatment based on the diagnosis performed at block 350. In some embodiments, the prescribed NF treatment is adjusted according to the type, stage and/or severity of the mental disorder of the subject. In some embodiments, prescribing the NF treatment for the subject comprises suggesting or advising the subject to perform the NF treatment, optionally without any previous diagnosis of a mental disorder in the subject. In some embodiments, a social worker and/or a therapist advises the subject to perform the NF treatment, for example as a treatment to mental, social, behavioral and/or cognitive difficulties of the subject.

According to some exemplary embodiments, the subject undergoes NF treatment sessions, at block 354. In some embodiments, the subject performs the NF treatment sessions in a treatment center and/or outside a treatment center, for example at home. In some embodiments, a NF treatment comprises one or more, for example 2, 4, 6, 10, 12, 14, 20 or any intermediate, smaller or larger number of treatment sessions. In some embodiments, the treatment sessions are performed with an interval of at least 1 hour, for example at least 6 hours, at least 12 hours, at least 24 hours, at least 48 hours, or any intermediate, smaller or larger time duration between two consecutive NF treatment sessions. In some embodiments, during a NF treatment session, the subject receives a feedback signal, optionally online, indicating an activity level of at least one specific brain region or at least one specific brain network.

According to some exemplary embodiments, the subject optionally performs one or more NF treatment maintenance sessions, at block 356. In some embodiments, the subject performs the NF treatment maintenance sessions, also termed herein as maintenance sessions, between the NF treatment sessions. In some embodiments, during the maintenance sessions the subject is instructed to modulate the activity of the at least one brain region or brain network, as instructed and trained during the NF training sessions. In some embodiments, during the maintenance sessions the subject does not receive a feedback signal regarding the activity of the at least one specific brain region or brain network. Alternatively, the subject receives feedback during maintenance session based on measurements of at least one physiological parameter, for example heart rate, blood pressure, or any measurable physiological parameter that is directly or indirectly affected by the activation level of the at least one brain region and/or brain network.

According to some exemplary embodiments, the subject is engaged in daily activities, for example between two NF treatment sessions and/or when finishing a NF treatment session.

According to some exemplary embodiments, the subject is optionally not feeling well, at block 358. In some embodiments, the subject feels an elevation or increase in one or more symptoms associated with a mental disorder and/or difficulties from which the subject suffers.

Alternatively, the subject optionally expects a difficulty, at block 360. In some embodiments, the subject expects a difficulty in the future due to an event expected to take place in the future. In some embodiments, the expected difficulty comprises behavioral, social, mental and/or cognitive difficulties.

According to some exemplary embodiments, the subjects optionally delivers a report at block 362. In some embodiments, the subject delivers a report regarding current feelings and/or current state. Alternatively or additionally, the subject delivers report regarding the expected difficulty. In some embodiments, the subject delivers a report to the system using a user interface of a remote device, for example remote device 210 shown in FIG. 3A, or via a personal device, for example personal device 214 shown in FIG. 3B, that is in communication with the remote device 210. In some embodiments, the subject delivers the report using an application software, which optionally serves as a user interface of the system. In some embodiments, delivery of the report comprises at least one of, filling one or more questionnaires, measuring at least one physiological parameter, and performing at least one assessment, optionally using the user interface.

According to some exemplary embodiments, the subject optionally receives instructions, at block 364. In some embodiments, the instructions comprise recommendations or suggestions. In some embodiments, the subject receives the instructions from the system via the user interface, for example a user interface of the remote device 210 or the personal device 214.

According to some exemplary embodiments, the instructions comprise instructions to perform one or more maintenance sessions, at block 366. In some embodiments, the instructions comprises instructions to modify one or more parameters of the maintenance session in view of the current feelings or current state, or in view of the expected difficulty. In some embodiments, the one or more parameter comprises at least one of, duration of a maintenance session or a portion thereof, timing of the maintenance session, for example when to perform the maintenance session, and/or content of the maintenance session.

According to some exemplary embodiments, the instructions comprise an invitation to arrive to a NF treatment session, at block 368. In some embodiments, the NF treatment session is rescheduled in view of the current feeling or current state of the subject, or in view of the expected difficulty.

According to some exemplary embodiments, the instructions comprise instructions to initiate a different treatment, for example a drug treatment, at block 370. In some embodiments, the instructions comprise instructions to take a drug, optionally previously prescribed to the subject. Alternatively or additionally, the instructions comprise instructions to perform at least one of, a psychological treatment, a behavioral treatment, a mental exercise, a cognitive exercise, and a motor exercise. Alternatively or additionally, the instructions comprises instructions to arrive to a meeting with an expert or a therapist. Optionally, the system schedules a meeting with the expert or therapist and the instructions comprise instructions to arrive to the scheduled meeting.

According to some exemplary embodiments, the subject reports on the subject state, optionally routinely, at block 372. In some embodiments, the subject reports according to a reporting scheduled determined by the system and/or an expert or a therapist which optionally monitors the NF treatment. In some embodiments, the subject provides the report regarding the subject state, during an interval period between NF treatment sessions. In some embodiments, if the subject report indicates a state or a change in the subject state that is not a desired state or a desired change, the subject receives instructions, for example as described at block 364.

According to some exemplary embodiments, the subject reports at block 372, as described at block 362, for example using a user interface of a remote device, for example remote device 210 shown in FIG. 3A, or via a personal device, for example personal device 214 shown in FIG. 3B, that is in communication with the remote device 210. In some embodiments, the subject delivers the report using an application software, which optionally serves as a user interface of the system. In some embodiments, delivery of the report comprises at least one of, filling one or more questionnaires, measuring at least one physiological parameter, and performing at least one assessment, optionally using the user interface.

Reference is now made to FIG. 3D, depicting a flow of activities performed by a subject using the system, after the NF treatment ends, according to some exemplary embodiments of the invention.

According to some exemplary embodiments, after the NF treatment ends at block 374, the subject optionally delivers reports to the system regarding the subject state, at block. In some embodiments, the subject delivers the reports as part of a planned routine, for example at least once a day, at least once a week, at least once in every two weeks, at least once a month, or any other smaller, intermediate or larger time period. In some embodiments, the subject delivers the report as described at block 372.

According to some exemplary embodiments, after the NF treatment ends at block 374, the subject optionally performs one or more maintenance sessions, at block 378. In some embodiments, the maintenance sessions are performed as described at block 356. In some embodiments, the maintenance sessions are optionally performed according to a predetermined routine or a predetermined plan, for example a post-treatment plan. In some embodiments, the post-treatment plan is determined by the system and/or an expert or a therapist. In some embodiments, the post-treatment plan includes actions the subject should perform after completing the NF treatment, for example when the subject is at home and/or is engaged in daily activities.

According to some exemplary embodiments, if the subject is optionally not feeling well at block 382 or expects a difficulty at block 380, the subject delivers a report at block 384. In some embodiments, the subject delivers the report at block 384, for example as described at block 362. According to some exemplary embodiments, based on the report delivered by the subject at block 384, the subject receives instructions at block 386. In some embodiments, the instructions comprise instructions to perform at least one maintenance session at block 392 and/or instructions to arrive to a booster NF treatment session at block 390, and/or instructions to take a drug, perform a different treatment and/or perform an exercise at block 388, for example as also described in block 370. Alternatively and/or additionally, the instructions optionally comprise instructions to arrive to a meeting with an expert or a therapist, at block 394. Optionally, the meeting is scheduled by or using the system.

### Exemplary Flow of System Activities

According to some exemplary embodiments, the system is configured to at least one of, deliver a NF treatment to a subject in a treatment center and/or outside the treatment center, monitor the subject state during the NF treatment, monitor the subject progress during the NF treatment, modify the NF treatment if needed, deliver a treatment on demand to the subject, be responsive to input from the subject, and provide suggestions and/or recommendations to the subject based on the input received from the subject and/or other input collected by the system.

Reference is now made to FIG. 3E, depicting a flow of activities performed by the system, according to some exemplary embodiments of the invention.

According to some exemplary embodiments, the system optionally checks suitability of a subject to a NF treatment, at block 353. In some embodiments, the system checks suitability of the subject to the NF treatment by reading signals, for example EEG signals from the subject brain while the subject performs at least part of a NF training session, for example NF training.

According to some exemplary embodiments, the system delivers NF treatment to the subject, at block 355. In some embodiments, the system delivers one or more NF training sessions to the subject.

According to some exemplary embodiments, the system monitors the subject performance during the NF treatment sessions, at block 357. In some embodiments, the system monitors the subject ability to modulate the activity of at least one specific brain region or at least one specific brain network, in a desired direction, optionally based on EEG signals measured from the subject brain.

According to some exemplary embodiments, the system monitors the subject state during the NF treatment, at block 359. In some embodiments, the system monitors the subject state, for example mental, cognitive and/or physiological state, during at least one or each NF training session. In some embodiments, the system monitors the subject state based on input received from the subject, for example questionnaires filled by the subject, measurements of electrical signals from the body of the subject for example EEG signals, and/or measurements of at least one physiological parameter. Alternatively or additionally, the system monitors the subject state based on information received by at least one optic sensor, for example a camera. Alternatively or additionally, the system monitors the subject state based on information received from an expert, a therapist, a caregiver, and/or a family member of the subject.

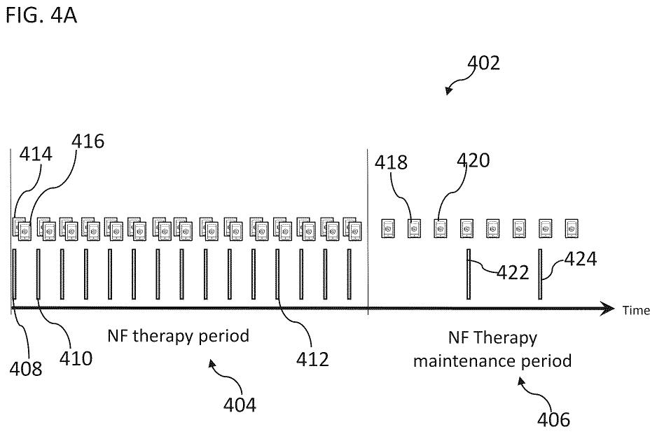

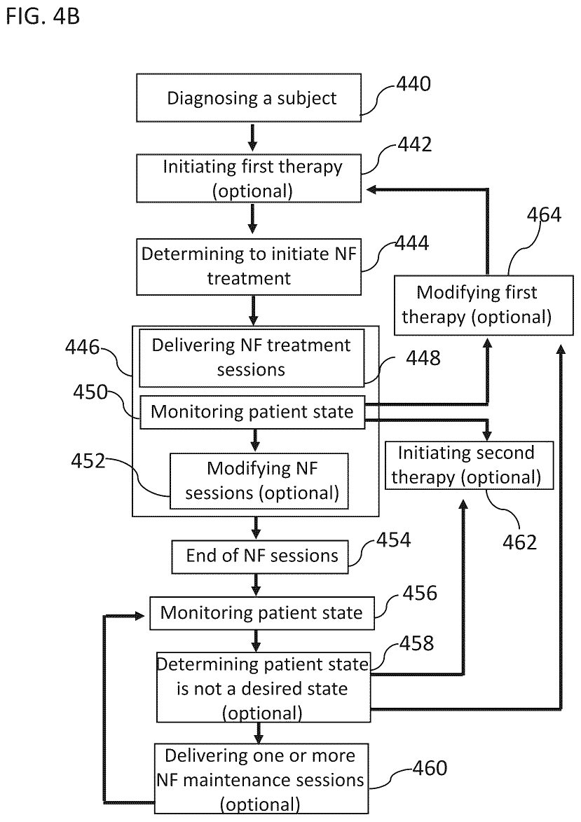

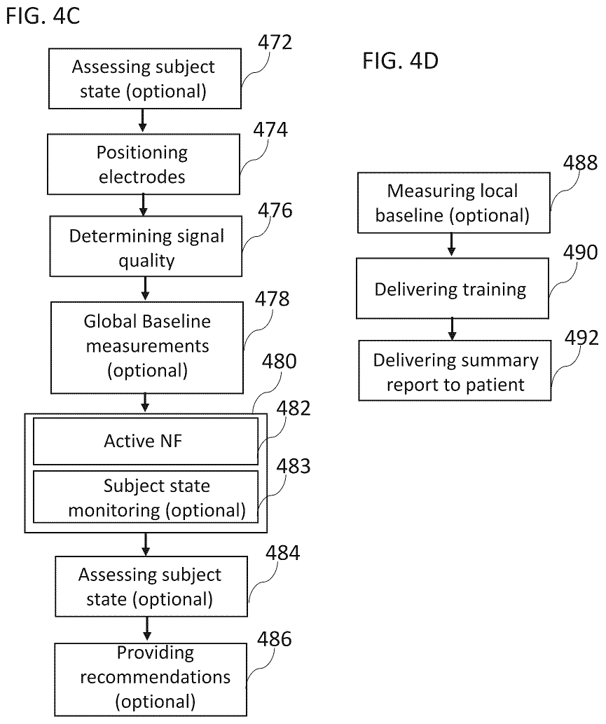

According to some exemplary embodiments, the system optionally modifies the NF treatment, for example at least one parameter of the NF treatment, at block 361. In some embodiments, the system modifies the NF treatment for example by modifying at least one of, duration of one or more treatment sessions, number of treatment sessions, content of one or more treatment sessions, duration between treatment sessions, duration and/or content of a maintenance session, sequence of blocks and/or duration of at least one block of a treatment session, for example the blocks shown in FIGS. 4C and 4D, and/or by shortening or stopping at least one treatment session or the entire NF treatment.

According to some exemplary embodiments, the system delivers one or more maintenance sessions, at block 363. In some embodiments, the system delivers one or more maintenance sessions to the subject, when the subject is outside the treatment center, for example when the subject is at home. In some embodiments, the maintenance sessions include one or more elements, for example features, that are shared with the NF treatment delivered to the subject, for example sound and/or visual interface presented to the subject. In some embodiments, the system provides an indication to the subject prior to initiation of a maintenance session and/or prior to an end of the maintenance.

According to some exemplary embodiments, if the system receives information that the subject cannot participate in a planned maintenance session, the system reschedules the maintenance session and delivers an indication to the subject and/or to a supervisor of the NF treatment, for example to an expert or to a therapist.

According to some exemplary embodiments, the system optionally receives indications regarding the subject state, at block 365. In some embodiments, the indications are based on information received directly from the subject, for example as described at blocks 362, 372 and 384 of FIGS. 3C and 3D, respectively. Alternatively or additionally, the system receives indications regarding the subject state, for example as described at block 376. In some embodiments, the system receives the indications when the subject is outside the treatment center, for example after the subject is released to his home or is at his home or work.

According to some exemplary embodiments, the system optionally sends instructions to the subject, at block 367. In some embodiments, the system sends instructions to the subject based on the indications received at block 365.

According to some exemplary embodiments, the instructions comprise instructions to perform at least one maintenance session at block 369. In some embodiments, the instructions comprise instructions to visit an expert or a therapist, optionally in a clinic, at block 371. In some embodiments, the instructions include taking a drug and/or performing at least one additional treatment, at block 373. In some embodiments, the instructions include modify a maintenance session and/or an additional treatment, at block 377. In some embodiments, the instructions include instructions to perform at least one additional NF session, for example a NF booster session.

### Exemplary NF Treatment

According to some exemplary embodiments, a NF treatment delivered to a patient is designed to train the patient how to self-control an activity of a specific brain region or an activity of a brain network, for example, a brain network that comprises the brain region or is related to the brain region. In some embodiments, self-controlling of the activity, for example upregulating or downregulating the activity allows, for example, to attenuate or to prevent an appearance of at least one symptom of a mental disorder.

According to some exemplary embodiments, the NF treatment includes one or more NF sessions in which the patient is instructed to apply at least one strategy, for example a cognitive task, while receiving feedback on an activity of the at least one brain region and/or the at least one brain network during the application of the strategy. In some embodiments, the received feedback allows the patient to find the best strategy for modulating the activity of the brain region and/or brain network in the desired direction. In some embodiments, feedback regarding the activity of the brain region or network is received by measuring EEG signals only, optionally using an electrical fingerprint of the brain region or network generated by correlating Blood Oxygenation Level Dependent (BOLD) activity of the brain region or network with a specific set of EEG signals. Reference is now made to FIG. 4A, depicting a general scheme of a NF treatment, according to some exemplary embodiments of the invention.

According to some exemplary embodiments, a NF treatment 402 comprises a NF therapy period 404, followed by a NF therapy maintenance period 406. In some embodiments, the NF therapy period 404 comprises two or more treatment sessions, for example treatment sessions 408, 410 and 412, over a time period of at least one week, at least two weeks, at least 4 weeks, at least 1 month, at least two months, at least 4 months, or any intermediate, shorter or longer time period. In some embodiments, the treatment sessions are conducted in a frequency of a treatment session per week, two treatment sessions per week, three treatment sessions per week, four treatment sessions per week, a treatment session per day, or any intermediate, smaller or larger number of treatment sessions per week. In some embodiments, the length of the NF therapy period and/or the number of treatment sessions during this time period is personalized for a specific subject, for example based on a state of the subject prior to the initiation of the NF treatment, and/or based on a state of the subject during the NF therapy period 404, based on a state of the subject during one or more treatment sessions 408, and/or based on the subject state between the treatment sessions.

According to some exemplary embodiments, during the NF therapy period 412, a personal device of the subject, for example personal device 214 shown in FIGS. 2 and 3B is used to receive input, for example feedback, from the subject, during a treatment session, for example at 414, and/or between treatment sessions, for example at 416. In some embodiments, the personal device 214 receives information form the subject using a software program installed in the memory of the personal device 214. In some embodiments, the personal device 214 receives information form the subject, for example information regarding the subject state, in an overt way and/or in covert way. In some embodiments, the personal device 214 receives information from the subject in an overt way, for example by instructing the subject to fill one or more questionnaires stored in the memory of the device 214. In some embodiments, the personal device 214 receives information from the subject in a covert way by at least one of, using signals from one or more sensors of the personal device or sensors of a device coupled to the personal device, using signals from a camera of the personal device or a camera coupled directly or indirectly to the personal device, by monitoring usage habits of the personal device 214 by the subject, by identifying words and/or phrases in conversations via the device 214, by monitoring written discussions or conversations and/or audio calls performed by the personal device 214, and/or using one or more sensors of the personal device 214.

According to some exemplary embodiments, the personal device 214 is used to receive information form the subject when the subject is at the treatment center during a NF treatment session, and/or when the subject is outside the treatment center, for example at home and/or is engaged in everyday activities.

According to some exemplary embodiments, after finishing the NF therapy period 404, the subject optionally enters a NF therapy maintenance period 406. In some embodiments, during period 406 the subject is at home and/or is engaged in everyday activities. In some embodiments, during period 406, the at least one software application stored in the memory of the personal device 214 is used to maintain the effect of the NF treatment, for example NF training, delivered in 404, in one or more maintenance sessions, for example sessions 418 and 420, for example by exposing the subject to a similar or an identical scenario as the scenario practiced during the sessions 408, 410 and 412, and instructing the subject to apply the strategy he used during the sessions 408, 410 and 412.

According to some exemplary embodiments, the maintenance sessions 418 and 420 are delivered according to a planned schedule, for example twice a day, once a day, twice a week, 3 times per week, or any smaller or larger number of maintenance sessions per week. Alternatively, the maintained sessions are delivered per demand, or according to information or signals transmitted to the personal device 214 from a remote device, for example remote device 210 shown in FIGS. 2, 3A and 3B.

According to some exemplary embodiments, during the maintenance period 406 the personal device 214 is used for receiving information from the subject, for example as described with regard to the therapy period 404, optionally to allow determining of the subject state. In some embodiments, the personal device 214 receives information from the subject before, during and/or after performing the maintenance sessions.

According to some exemplary embodiments, a state of the subject is determined during the period 406 based on the information received by the personal device. Alternatively or additionally, a capacity of the NF therapy effect during the period 406 is determined based on the information received by the personal device. In some embodiments, if the determined NF therapy effect capacity is lower than and/or above a predetermined level, the subject is invited to one or more additional NF therapy sessions, for example booster NF sessions 422 and 424. In some embodiments, during the booster sessions 422 and 424, a NF therapy session is delivered to the subject in a treatment center, or outside the treatment center, for example when using a portable NF device.

According to some exemplary embodiments, the personal device 214 is used to monitor a state of the subject after the NF therapy period 404, and to instruct the subject to at least one of, participate in one or more NF booster sessions, schedule a meeting with a therapist, for example a psychologist or a Psychiatrist, stop administering at least one drug, initiate administering of at least one drug, optionally based on the subject state and/or based on the determined NF capacity.

Reference is now made to FIG. 4B depicting a general NF treatment process, according to some exemplary embodiments of the invention.

According to some exemplary embodiments, a subject is diagnosed at block 440. In some embodiments, the subject is diagnosed with a mental disorder according to standard classification of mental disorders used by mental health professionals, for example according to the Diagnostic and Statistical Manual of Mental Disorders (DSM).

According to some exemplary embodiments, at least one first treatment is optionally initiated, at block 442. In some embodiments, the at least one first treatment comprises at least one of, a drug treatment, a psychological treatment, a psychotherapy or CBT or Dialectical Behavior Therapy (DBT) treatment, an Electroconvulsive therapy (ECT). In some embodiments, the first treatment is selected and initiated according to the diagnosed mental disorder.

According to some exemplary embodiments, an expert determines to initiate a NF treatment in the subject, at block 444. In some embodiments, the expert determines to initiate the NF treatment based on the diagnosis performed at block 440, and/or based on the diagnosed mental disorder. Optionally, the expert determines to initiate the NF treatment as a complementary treatment to at least one first treatment, for example when the first treatment alone does not yield desired results. Optionally, the expert determines to initiate the NF treatment as a replacement to at least one first therapy.

According to some exemplary embodiments, during the NF treatment 446, the subject participates in one or more, for example 2, 3, 4 5, 10, 15, 20 or any intermediate, smaller or larger number of treatment sessions, at block 448. In some embodiments, the NF treatment sessions are delivered in a treatment center, for example as described in FIGS. 1 and 4A.

According to some exemplary embodiments, during the NF treatment 446 and/or during the NF treatment sessions 448, a state, for example mental, behavioral, and/or cognitive state of the subject is monitored, at block 450. In some embodiments, the state of the subject is monitored using information and/or feedback received from the subject. Alternatively or additionally, the subject state is monitored based on signals and/or measurements recorded or received from the subject. In some embodiments, the subject state is monitored, for example as described in FIGS. 1, 3A and 3B.

According to some exemplary embodiments, at least one second treatment is optionally initiated at block 462. Optionally, the at least one second treatment is optionally initiated base on the results of the monitoring performed at block 450. In some embodiments, the at least one second treatment comprises at least one of, a drug treatment, a psychological treatment, a CBT treatment, an Electroconvulsive therapy (ECT). In some embodiments, the first treatment is selected and initiated according to the diagnosed mental disorder.

According to some exemplary embodiments, the at least one first treatment is optionally modified at block 464. In some embodiments, the at least one first treatment is modified based on the results of the monitoring performed at block 450. In some embodiments, at least one parameter for the first treatment is modified, for example administration regimen of a drug, dose of a drug, number of drugs, type of drugs, and/or number of sessions of the first treatment.

According to some exemplary embodiments, one or more parameters of the NF treatment sessions are optionally modified based on the results of the monitoring, at block 452. In some embodiments, the one or more of the NF treatment sessions parameters are modified based on the monitored subject state. In some embodiments, the one or more NF treatment parameters comprise at least one of, a length of at least one treatment session, a starting difficulty level of at least one treatment session, interval length between two treatment sessions, number of treatment sessions, scheduling details of at least one treatment session, type of interface presented to the subject in a treatment session, number, timing and/or order of blocks within a treatment session, number and/or position of electrodes used in at least one treatment session, number and/or type of an electric fingerprint (EFP) and/or NF treatment protocol.

According to some exemplary embodiments, modifying NF treatment sessions comprises, stopping at least one treatment session or stopping overall treatment.

According to some exemplary embodiments, the NF treatment sessions end at block 454. In some embodiments, the NF therapy period, for example therapy period 404 described in FIG. 4A, ends at block 454. The length of the NF therapy period 404 is determined at block 444. Optionally, the length of the NF therapy period is modified during the therapy period, for example during the NF treatment 446, optionally, based on the subject state, for example as monitored at block 450.

According to some exemplary embodiments, the subject state is monitored following the end of the NF treatment, at block 456. In some embodiments, the subject state is monitored as described at block 450. In some embodiments, the subject state is monitored according to a specific monitoring schedule. Alternatively, the subject state is monitored on-demand, for example when receiving a signal from the remote device, for example the remote device 210 shown in FIGS. 2, 3A and 3B. In some embodiments, the subject state in monitored at block 456 using a personal device, for example personal device 214. In some embodiments, the subject state is monitored at block 456 when the subject is outside the treatment center, for example when the subject is at home or is engaged in daily activities.

According to some exemplary embodiments, the patient state is determined to be a non-desired state, at block 458. In some embodiments, a non-desired state is a state that is not a target state, for example a target state according to a treatment plan and/or a target state according to known standards in the field of mental disorders treatment and/or diagnosis. In some embodiments, a non-desired state is a state that is not normal according to standards known in the field of mental disorders treatment and/or diagnosis.

According to some exemplary embodiments, if the subject state is not a target state or that the subject state indicates a deterioration of the subject state relative to a reference state, at least one NF maintenance session is optionally delivered to the subject at block 460. In some embodiments, at least one NF maintenance session comprises a session in which the subject is presented with an interface, for example a visual and/or an audio interface, and is instructed to apply a mental strategy. In some embodiments, the mental strategy is a strategy that was shown to modulate an activity of at least one brain region or at least one brain network in the subject during active neurofeedback sessions in the subject or in other subjects. Alternatively or additionally, the interface displayed to the subject during NF maintenance is an interface presented to the subject during the active neurofeedback sessions.

According to some exemplary embodiments, if the subject state is not a target state, then the at least one first therapy is optionally modified at block 464, for example as described above. According to some exemplary embodiments, if the subject state is not a target state, then the at least one second therapy is optionally initiated at block 462, for example as described above. Alternatively, at least one second therapy is optionally modified.

### Exemplary NF Treatment Session

According to some exemplary embodiments, a NF treatment includes at least one, for example two or more NF treatment sessions occurring at different times. In some embodiments, each NF treatment session is performed in a treatment center. Optionally, at least one, some or all of the NF treatment sessions are performed at home or outside a treatment center. In some embodiments, during the NF treatment sessions, a subject received feedback on an activity of at least one brain region or at least one brain network based on EEG signals measured from the subject head. In some embodiments, the subject receives the feedback in less than 1 minute, for example less than 30 seconds, less than 15 seconds, less than 10 seconds, less than 5 seconds, or any intermediate, smaller or larger time period from measuring the EEG signals.

Reference is now made to FIG. 4C, depicting a process for performing a NF treatment session, according to some exemplary embodiments of the invention.

According to some exemplary embodiments, a subject, for example a patient previously diagnosed with a mental disorder, optionally arrives at a treatment center. Alternatively the subject is optionally outside the treatment center, for example the subject is at his home.

According to some exemplary embodiments, a state of the subject is optionally assessed at block 472. In some embodiments, the state of the subject, for example mental and/or cognitive state, is assessed when the subject is at the treatment center. Alternatively the state of the subject is assessed before the subject arrives to the treatment center, for example when the subject is still at home.

According to some exemplary embodiments, the state of the subject is optionally assessed at block 472, by receiving input, for example information from the subject. In some embodiments, the subject state is assessed, for example by performing one or more questionnaires using a NF device at the treatment center and/or using a personal device of the subject. Alternatively or additionally, the subject state is assessed using one or more sensors, for example sensors of the NF device, sensors of a personal device and/or sensors of a device that is in communication with the NF device and/or with the subject personal device. In some embodiments, the subject state is assessed at block 472 as previously described in FIGS. 1, 2, 3A, 3B and 4B.

According to some exemplary embodiments, the subject state is optionally assessed at block 472, for example for establishing a baseline or a reference state. Alternatively or additionally, the subject state is optionally assessed at block 472 for determining if the subject is capable, for example from a mental and/or a cognitive perspective, to participate in a NF treatment session.

According to some exemplary embodiments, the subject state is optionally assessed at block 472 automatically, for example by automatically delivering instructions to the subject. Alternatively, the subject state is optionally assessed based on signals received from an expert, for example a therapist who is responsible and/or monitors the NF treatment session.

According to some exemplary embodiments, one or more electrodes are coupled to the subject body, at block 474. In some embodiments, the one or more electrodes are coupled to the subject head and are configured to record signals from the subject brain. In some embodiments, the one or more electrodes for example electrodes 316 and 318 shown in FIG. 3A are coupled, for example attached to the subject scalp. In some embodiments, the one or more electrodes are positioned on a subject head at specific locations, optionally according to an EEG coordinate system, for example a 10-20 coordinate system or variations thereof. In some embodiments, at least one electrode, for example a reference electrode is coupled to the subject head, for example to an ear or an ear lobe of the subject. Optionally, a conductive material, for example gel is applied to the electrodes. Optionally, the electrodes are coupled to the subject head using an alignment cap which optionally has openings based on the EEG coordinate system.

According to some exemplary embodiments, signal quality is determined at block 476. In some embodiments, determining signal quality comprises determining that the one or more electrodes are in contact with the head, for example with the scalp of the subject. Alternatively or additionally, determining signal quality comprises determining that the contact between the one or more electrodes with the body of the subject, for example with the scalp of the subject, is sufficient to record signals that can be used during the NF session, optionally for EEG measurements. Alternatively or additionally, determining signal quality comprises determining that a signal generated by the one or more electrodes is suitable for EEG measurements. Optionally, determining signal quality comprises determining that wiring between the electrodes to a NF device and/or to an amplifier is connected and operated in a way that allows recording of electrical signals by the one or more electrodes.

According to some exemplary embodiments, a global baseline is optionally measured at block 478. In some embodiments, the global baseline is measured while a non-relevant and/or a static interface, for example an audio and/or a visual interface is provided to the subject. In some embodiments, the interface is displayed to the subject. In some embodiments, the interface is an interface that will not be used during actual NF training and/or is an interface that is not relevant and/or is not in a context of the subject mental disorder and/or the interface is a static interface that does not change during the global baseline measurement. In some embodiments, the non-relevant interface is presented to the subject during a time period in a range between 10 seconds and 5 minutes, for example during a time period between 1 minute and 3 minutes, a time period between 2 minutes and 3 minutes, or any intermediate, smaller or larger time period.

Optionally or alternatively, the global baseline measurements are used to determine the state of the subject.

According to some exemplary embodiments, during global baseline measurements at block 478, a feedback regarding the activity level of at least one brain region or at least one brain network or biomarker thereof, is not delivered to the subject. In some embodiments, the measured global baseline is a baseline of a signal, for example an electrical fingerprint signal (EFP), that indicates a baseline activity level of the at least one brain region and/or the at least one brain network when the subject is at rest and is not engaged in process that tries to modulate the activity of the at least one brain region or the at least one brain network, or biomarker thereof. In some embodiments, the EFP signal is based on EEG measurements generated from EEG signals recorded from the subject that were processed using a model (the EFP) that allows to correlate EEG signals recorded from a specific set of electrodes, having a specific frequency range or specific frequency ranges, during a specific time widows, with fMRI-BOLD activity of the at least one specific brain region or the at least one specific network.

According to some exemplary embodiments, the global baseline is, or is generated, based on at least one central tendency measurement of the EFP or EEG signals measured at block 478. In some embodiments, the at least one central tendency measurement comprises at least one of, mean, median, and/or mode of the measured EFP or EEG signal. Optionally, the central tendency can be extracted for each frequency band in correspondence to the frequency bands used in the generation of the EFP. In some embodiments, the global baseline is measured while the subject looks at a fixed visual indication, for example a fixed image of a cross, alternatively, the global baseline is measured while the subject sits with eyes closed until a tone signal is played indicating the end of the global baseline.

According to some exemplary embodiments, once a global baseline signal is optionally measured, one or more sessions 480 of active NF is delivered to the subject. In some embodiments, each session 480 comprises a session of active neurofeedback training 482. In some embodiments, during the one or more sessions 480, for example during the active NF training 482, the subject state is monitored at block 483. In some embodiments, the subject state is monitored and/or assessed based on at least one of, behavior of the subject during training 482, signals received from one or more sensors, for example optic sensors or EMG sensors, EEG signals measured from the subject brain during the training 482, feedback received from an expert, for example a therapist monitoring the NF treatment session.

According to some exemplary embodiments, the training 482 is modified based on the subject state assessed at block 483, for example, training 482 is stopped or shortened if subject state assessment at block 483 indicates that the subject is not sufficiently engaged in the NF training 482 or has lack of interest in proceeding with the training 482. Alternatively or additionally, the training 482 is stopped if the patient state assessment at block 483 indicates appearance of one or more symptoms of a mental disorder during the training 482, and/or if the cognitive and/or mental state of the subject is not a desired, for example a target state when performing the training 482. According to some exemplary embodiments, after completing the active NF sessions 480, the state of the subject is optionally assessed, at block 484. In some embodiments, the subject state is assessed, for example as described at block 472. In some embodiments, the state of the subject is optionally assessed at block 484, for example how and/or to which extent, the active NF sessions performed at block 480 affected the subject state, optionally in comparison to the subject state assessed at block 472.

According to some exemplary embodiments, suggestions, for example recommendations, are optionally provided to the subject at block 486, optionally, at the end of the NF treatment session. In some embodiments, the suggestions include suggestions regarding to the next NF treatment sessions and/or suggestions regarding performing activities outside the treatment center and/or between consecutive NF treatment sessions. Alternatively or additionally, the suggestions provided at block 486 include suggestions regarding management of a therapeutic process outside the treatment center and/or following the NF treatment session, for example instructions regarding behavior when experiencing at last one symptom of the mental disorder and/or when expecting to encounter a trigger of the mental disorder or a trigger of a mental disorder symptom.

Reference is now made to FIG. 4D, depicting an active training process delivered at block 480, according to some exemplary embodiments of the invention.

According to some exemplary embodiments, during active NF sessions, one or more NF training sessions or cycles 482 are performed, for example in each NF treatment session described in FIG. 4C, a subject undergoes 2, 3, 4, 5, 6, 7, 8, 9, 10 or any larger number of active NF training sessions 482, optionally according to a predetermined treatment plan or according to modifications of a predetermined treatment plan.

According to some exemplary embodiments, each active NF training cycle optionally initiates with measurements of a local baseline, at block 488. In some embodiments, measurements of a local baseline are used to calculate a baseline or a reference value of the EFP signal or EEG signal, indicating the activity of the at least one brain region or brain network or biomarker thereof. In some embodiments, the local baseline is optionally used in the specific NF training cycle. Alternatively, a single local baseline may be used as a reference for two or more NF training cycles. According to some exemplary embodiments, the local baseline is measured and optionally set when the subject is presented with an interactive audio/visual interface, optionally displaying avatars. In some embodiments, the audio/visual interface is the interface that will be used during NF training. In some embodiments, the subject is instructed to watch the interface but not to attempt to control it. In some embodiments, during block 488 the interface does not respond according to an EEG signal measured from the subject or changes thereof. In some embodiments, during block 488 feedback regarding the activity of the at least one brain region or the at least one brain network is not delivered to the subject, for example by modifying the interface according to the determined activity of the brain region or brain network.

Alternatively, the global baseline measurements are used instead of the local baseline.

According to some exemplary embodiments, NF training is delivered at block 490. In some embodiments, during NF training, the interface, for example the audio/visual interface is modified according to the activity of the at least one brain region or brain network, optionally based on changes in the EEG signal or EFP signal, during training. In some embodiments, during the NF training, the subject is instructed to try and modify the presented interface by applying one or more strategies, for example mental or cognitive strategies. In some embodiments, applying the one or more mental strategies comprises performing at least one cognitive or mental task while providing feedback, for example covert feedback, to the subject regarding the activity of the at least one brain region or brain network by modifying the audio/visual interface. In some embodiments, during the NF training at block 490, the subject learn to regulate the activity of the at least one brain region or brain network by applying a strategy, for example a strategy that is personalized to the specific subject. In some embodiments, a duration of blocks 488, 490 is within a range of 30 seconds to 6 minutes, for example within a range between 30 seconds and 2 minutes, within a range between 1 minute and 3 minutes, within a range between 2 minutes and 4 minutes, or any intermediate, smaller or larger range of time duration. In some embodiments, a duration of block 492 is within a range between 10 seconds and 3 minutes, for example within a range between 10 seconds and 1 minute, within a range between 30 seconds and 2 minutes, or any intermediate, smaller or larger range of time duration.

According to some exemplary embodiments, summary report is delivered to the subject, at block 492. In some embodiments, during the summary report block, the delivers, for example presents, to the subject a summary optionally comprising a score for the training performed at block 492. In some embodiments, the score is calculated based on changes in the EFP signal or EEG signal measured during the NF training at block 490, optionally relative to the local baseline measured at block 488. Optionally, the calculated score is a central tendency measurement of the changes in the EFP or EEG signal in a desired direction, for example up or down, during the training at block 490, and optionally relative to the local baseline optionally measured at block 488.

### Exemplary Maintenance Session

According to some exemplary embodiments, for example as shown in FIG. 4A, one or more maintenance sessions, for example sessions 418 and 420 are performed, as part of the overall NF treatment 402. In some embodiments, the maintenance sessions are configured to prolong a therapeutic effect generated by active neurofeedback treatment sessions or of other therapies provided to the patient, during the NF therapy period 404. In some embodiments, the maintenance sessions are performed outside the treatment center, for example when the subject is at home and/or is engaged in daily activities. In some embodiments, the maintenance sessions are performed using a software installed in a device of the subject, for example a personal device comprising at least one of, a cellular device, a mobile device, a tablet. In some embodiments, the device of the subject comprises device 214 shown in FIGS. 2 and 3B.

According to some exemplary embodiments, the maintenance sessions are performed while the device of the subject is in communication with a remote device which includes details, for example one or more parameters of the maintenance sessions, for example remote device 210 shown in FIGS. 2, 3A and 3B. Alternatively, the maintenance sessions are performed while the personal device 214 is offline, for example not in communication with the remote device 210.

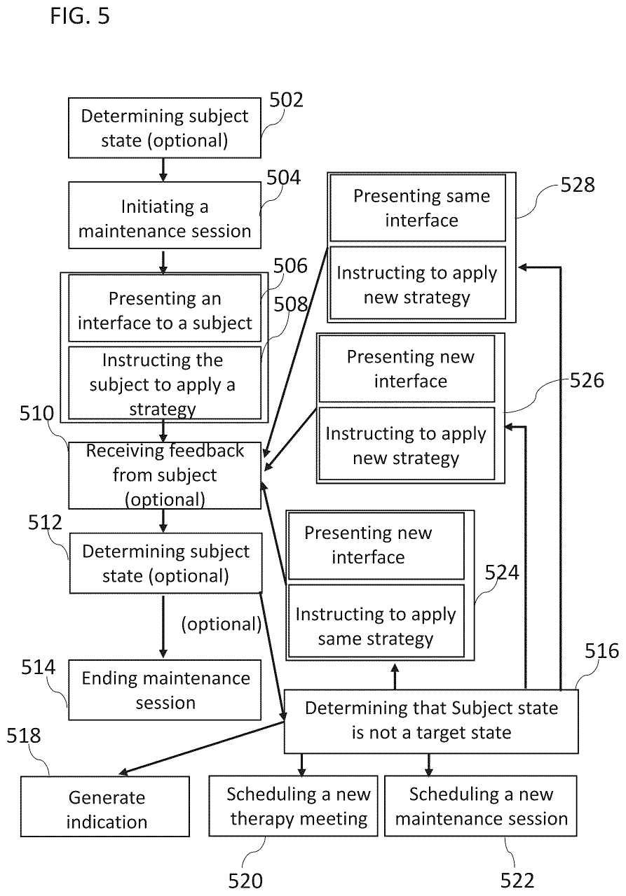

Reference is now made to FIG. 5 depicting a process for performing NF maintenance sessions, according to some exemplary embodiments of the inventions. In some embodiments, the NF maintenance sessions are performed by a control circuitry of a device, for example device 214, using a set of instructions, included in a maintenance software or a maintenance software application installed in a memory of the device.

According to some exemplary embodiments, at least one of, parameter values, timing and/or schedule, of a maintenance session are predetermined, for example according to NF treatment plan. In some embodiments, the NF treatment plan is generated by an expert and/or by a remote device, for example remote device 210. In some embodiments, the NF treatment plan and/or the maintenance sessions are personalized for a specific subject or a group of subjects.

According to some exemplary embodiments, the subject receives a reminder from the device, for example the personal device that a maintenance is about to initiate. In some embodiments, the subject can confirm his availability for the maintenance session or to reschedule the maintenance session. In some embodiments, if the subject reschedules the maintenance session, the maintenance software in the device memory provides a new date and/or a new time for the maintenance session. Optionally, the maintenance software sends an update to the remote device with the decision of the subject and/or with the new date and/or time for the maintenance session.

According to some exemplary embodiments, in the beginning of a maintenance session, a state of the subject is optionally determined at block 502. In some embodiments, the state of the subject is determined in order to make sure that a current state of the subject is a desired state or a target state, for performing the maintenance session, or has not been deteriorated to a state that does not allow performing of the maintenance session. In some embodiments, if the determined state is not a desired state, the maintenance session is rescheduled and/or an indication is generated. In some embodiments, the indication is transmitted to the expert and/or to the remote device 210. Alternatively, if the state of the patient is determined to be a desired state, the maintenance session is initiated at block 504.

According to some exemplary embodiments, the maintenance session is initiated at block 504.

According to some exemplary embodiments, during the maintenance session, an interface, for example maintenance interface is presented to the subject, at block 506. In some embodiments, the maintenance interface comprises an audio and/or a visual interface. In some embodiments, a control circuitry of the device presents the interface according to instructions and/or a maintenance protocol installed in the memory of the device, optionally as part of the maintenance software. In some embodiments, the interface presented at block 506 is the same interface or variations thereof, used during the active NF training sessions, for example sessions 482 shown in FIG. 4C. Alternatively the maintenance interface is an interface, for example that is different from the interface used for the NF training sessions 482.

According to some exemplary embodiments, during the presentation of the maintenance interface, the device delivers instructions to the subject to apply the strategy he used during the NF training sessions 482 that allowed him to modulate the activity of the at least one brain region or brain network, in a desired direction. In some embodiments, the instructions are delivered to the subject at block 508 before presenting the interface to the subject at block 506.

According to some exemplary embodiments, information, for example feedback signal, is optionally received from the subject at block 510. In some embodiments, the information is received using a user interface of the device, which is optionally part of the maintenance software. Alternatively, or additionally, the feedback signal is optionally received using one or more sensors of the device, for example an optic sensor, optionally a camera. Alternatively or additionally, the feedback signal is optionally received using sensors of one or more devices, for example wearable devices, in communication with the device of the subject used for the maintenance sessions. In some embodiments, the feedback signal is received for example as described in FIG. 3B, showing devices 340 and 342 that are in communication with the device 214. In some embodiments, feedback is provided by the subject at block 510 after providing instructions to the subject to fil one or more questionnaires.

According to some exemplary embodiments, the feedback signal is optionally received while or after the interface is presented to the subject at block 506.

According to some exemplary embodiments, a state of the subject is optionally determined at block 512. In some embodiments, the state of the subject is optionally determined based on the feedback received at block 510. Alternatively, the state of the subject is optionally determined as previously described at block 456 shown in FIG. 4B.

According to some exemplary embodiments, if the state of the subject at the end of the maintenance session is not a desired state, for example a target state, an indication is generated at block 518. Optionally, the indication is transmitted to a remote device. Alternatively or additionally, at least one new NF therapy session, for example at least one booster session, for example booster sessions 422 and 424, is scheduled. Alternatively or additionally, at least one new maintenance session is scheduled at block 522.

According to some exemplary embodiments, if the state of the subject at the end of the maintenance session is not a desired state, for example a target state, at block 516, the maintenance session is repeated by presenting a new interface to the subject and instructing the subject to apply the same strategy, at block 524. Alternatively, the maintenance session is repeated by presenting a new interface to the subject and instructing the subject to apply a new strategy, at block 526. Alternatively, the maintenance session is repeated by presenting the same interface as in block 506 to the subject and instructing the subject to apply a new strategy, at block 528.

Optionally, when scheduling a new maintenance session in case the subject state is not a target state, the presented interface and/or the strategy to be applied during the maintenance session, are modified.

### Exemplary Electrical Fingerprint (EFP)

Relative to functional magnetic resonance imaging (fMRI), used for detecting BOLD activity of brain regions, electroencephalogram (EEG) constitutes a much more scalable recording device. However, EEG has low spatial resolution, and therefore cannot detect activity originating from deep brain regions, for example regions of the limbic system, regions of the positive valence system and regions of the negative valence system.

To combine the advantages of the two measurement methods (fMRI and EEG), a tool was developed to predict fMRI BOLD activity in specific deep brain regions (such as the amygdala) based on EEG alone. After simultaneous recording of EEG and fMRI signals, advanced data registration and statistical techniques were applied on the recorded signals to correlate data from the two modalities. The result was a prediction model, also termed EFP, that now allows conversion of an EEG signal into a surrogate indicator of the BOLD fMRI signal in specific brain regions. An EFP of the amygdala is described in International Patent Application Publication number WO2012104853A2, incorporated herein as a reference in its entirely. An EFP of the Ventral Striatum is described in International Patent Application Publication number WO2021260697A1 incorporated herein as a reference in its entirely.

According to some exemplary embodiments, the EFP model is a weight coefficient matrix, optionally of at least 100 coefficients, for EEG transformed to time delay x frequency obtained from statistical correlation between simultaneously acquired EEG and fMRI recordings with a volume of interest (VOI) covering a specific brain region. In some embodiments, the coefficient matrix corresponds to frequency bands, electrodes and one or more time windows.

For example, an EFP of the Ventral Striatum comprises electrical signals, for example EEG electrical signals recorded from EEG electrodes located at positions C4, F7, F8, T7, T8, P8, TP9 and TP10. In some embodiments, the EFP comprises EEG electrical signals in a frequency range between 0 -40 Hz, and in a time delay window between 0 and 30 seconds. Alternatively, an EFP of the Ventral Striatum comprises electrical signals recorded from EEG electrodes located at positions C4, C3, Cz, P3, P4, Pz, filtered in a bandpass filter between 0.5-40 Hz, in a time delay window that range between 0 and 13.1 seconds.

### Exemplary Generation of an EFP Signal

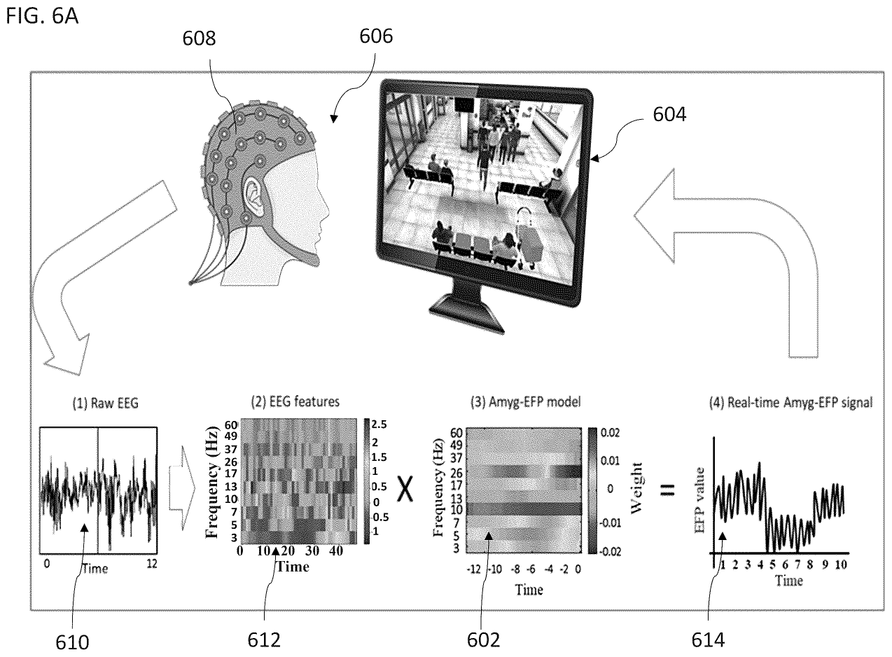

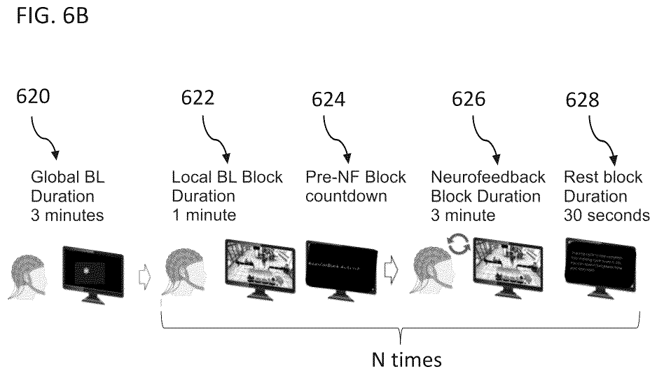

Reference is now made to FIG. 6A, depicting generation of an EFP signal, according to some exemplary embodiments of the invention. It should be noted that although FIG. 6A depicts a generation of an Amygdala-EFP signal predicting BOLD activity of the Amygdala, using an Amygdala-EFP model, generation of other signals predicting BOLD activity of other brain regions, for example deeply located brain regions, is performed in a similar way using an EFP model that is specific to the brain region.

According to some exemplary embodiments, during active NF sessions, for example session 482, an interface, for example a dynamic audio/visual interface 604 is displayed to the subject 606. Optionally, the subject 606 is instructed to try and modify dynamic audio/visual interface 604, for example by applying at least one strategy, for example by self-performing at least one cognitive and/or mental task.

According to some exemplary embodiments, electrical signals are recorded by one or more electrodes 608 positioned on the subject head, optionally, during the display of the dynamic audio/visual interface 604 to the subject 606. In some embodiments, the electrodes, are electrodes 316 and 318 shown in FIG. 3A.

According to some exemplary embodiments, the NF device measures at least one EEG signal, for example a raw EEG signal 610, from the recorded electrical signals.

According to some exemplary embodiments, EEG features 612 are extracted from the raw EEG signal 610. In some embodiments, the EEG features comprise frequency bands, and time delays for each electrode. Optionally, the features and intensities are arranged in a matrix.

According to some exemplary embodiments, the EEG features and their intensities are multiplied by the weights of the EFP model 602, to calculate an EFP signal 614 which indicates a prediction of BOLD-activity of the specific brain region or a specific network. Optionally, the specific brain region is part of the specific brain network.

According to some exemplary embodiments, the NF device, for example a control circuitry of the NF device is configured to modify at least one parameter of the interface 604 according to changes in the calculated EFP signal 614 In some embodiments, a feedback regarding the activity of the at least one brain region or the at least one brain network is delivered to the subject 606 by the interface 604 modifications. Alternatively or additionally, a feedback regarding the ability of the subject to modulate the activity of the at least one brain region or the activity of the at least one brain network is delivered to the subject 606 by the interface 604 modifications.

### Exemplary Active NF Cycle

Reference is now made to FIG. 6B, depicting an active NF cycle process, according to some exemplary embodiments of the invention.

According to some exemplary embodiments, one or more active NF cycles are performed during a NF treatment session, for example an active NF cycle 482 described in FIGS. 4C and 4D.

According to some exemplary embodiments, a global baseline is measured in each active NF cycle or at least one time during a NF treatment session. In some embodiments, in a global baseline measurement stage 620, EEG data is recorded during rest, for example while the subject is presented with a black screen with a white asterisk. Alternatively, the subject is presented with a non-dynamic interface and/or with an interface that is not relevant to the subject mental disorder and/or that cannot trigger one or more symptoms of the mental disorder. In some embodiments, the EEG is then preprocessed to remove noise artifacts. In some embodiments, following noise artifacts removal, a Stockwell transform is applied and its output is resampled, in both the time and frequency domains (to 4 Hz up to 250 Hz and 9 to 10 pre-defined frequency bands). In some embodiments, a central tendency measure, for example an average value for each frequency band is calculated and stored for the rest of the NF treatment session, where optionally they are used to center the time-frequency transformed EEG signals during the EFP signal computation.

According to some exemplary embodiments, after measurements of the global baseline, the subject is presented with a dynamic feedback interface, for example an animated feedback interface, for N times, for example for at least two times. In some embodiments, the subject is presented with the dynamic feedback interface for 3 times, for 4 times, for 5 times, for 6 times, for 7 times, for 8 times, or any larger number of times, repeating cycles of “rest”, when the subject is instructed not to try and modulate the dynamic feedback interface, and “regulate,” where the subject is instructed to try and modulate the dynamic feedback interface, optionally by applying a mental or a cognitive strategy.

According to some exemplary embodiments, during “rest” phases, for example phase 622, 622 and 628, EEG data is recorded while the subject watches the dynamic feedback interface, which in this phase does not respond or being modified to changes in the EEG signal, for example EFP signal (i.e. no feedback). Optionally, a local baseline 622 is measured, for example calculated during “rest” phase 622. In some embodiments, the EFP values are computed for this phase by calculating the resampled Stockwell transform, subtracting the average values calculated from the signal acquired during the Global Baseline phase, and multiplying the result by weights of the EFP model, for example EFP model 602.

According to some exemplary embodiments, EFP values are computed in the same way, as described above with respect to “rest” phase 622, during the active feedback phase 626, termed “regulate.” In some embodiments, a change in the calculated EFP signal between “regulate” and “rest” is then calculated using a summary statistics (mean and standard deviation) of the EFP values calculated during “rest” (Local Baseline). In some embodiments, this calculated change in the EFP signal is then reflected to the subject as a change in the dynamic feedback interface, for example the animated audio-visual interface.

According to some exemplary embodiments, a duration of a global baseline measurement phase 620 is within a range between 5 seconds and 5 minutes, for example in a range between 1 minute and 5 minutes, in a range between 2 minutes and 4 minutes, or any intermediate, shorter or longer time period.

According to some exemplary embodiments, a duration of a local baseline measurement phase 622, for example “rest” phase 622, is within a range between 5 seconds and 5 minutes, for example in a range between 1 minute and 5 minutes, in a range between 30 seconds and 2 minutes, or any intermediate, shorter or longer time period.

According to some exemplary embodiments, a duration of an active NF phase 626, is within a range between 5 seconds and 5 minutes, for example in a range between 1 minute and 5 minutes, in a range between 30 seconds and 2 minutes, or any intermediate, shorter or longer time period.

According to some exemplary embodiments, a duration of a “rest” phase 628, at an end of NF phase 626, is within a range between 5 seconds and 2 minutes, for example in a range between 1 minute and 5 minutes, in a range between 30 seconds and 2 minutes, or any intermediate, shorter or longer time period.

### Exemplary Signal Quality Monitoring

According to some exemplary embodiments, after the coupling of the one or more electrodes to the subject body, for example to the subject head, a quality of the signal recorded by the electrodes is determined. In some embodiments, the signal quality is determined prior to initiating the NF treatment session, and is optionally monitored during the NF treatment session, for example to make sure that the signal quality is sufficient for performing the NF treatment session. In some embodiments, if the signal quality is not sufficient the system automatically stops the NF treatment session. Alternatively, the system delivers instructions or suggestions to stop the NF treatment session and/or instructions of suggestions how to improve signal quality.

In some embodiments, the term “quality of a signal” and derivatives correlate with a level of a noise signal recorded by the electrode. An electrode which records a high quality signal introduces less noise into the recorded signal.

According to some exemplary embodiments, the signal quality is determined and monitored per each electrode positioned on the subject head. In some embodiments, the NF system, for example the NF system 302 shown in FIG. 3A, or a NF device, delivers feedback to the subject or to an expert, for example a supervisor, regarding the signal quality of each or all electrodes. Optionally, the feedback information is color coded feedback and/or a value, indicating a quality level of the recorded signal or the suitability of at least one electrode to be used during the NF treatment session. Optionally, the system presents the user with instructions on how to improve the signal quality.

According to some exemplary embodiments, quality of a signal recorded by one or more electrodes, or all of the electrodes is determined relative to an absolute reference value or to absolute minimum and maximum values of a reference range. Alternatively or additionally, quality of a signal recorded by one or more electrodes, or all of the electrodes is determined relative to at least one selected electrode which records a signal with a target quality level. In some embodiments, the system determines signal quality in at least one first stage in which quality of a signal recorded by each electrode is determined relative to at least one absolute reference value, and in at least one second stage in which quality of the recorded signal is determined relative to a signal recorded by at least one selected electrode which exhibited a target quality level. Alternatively or additionally, quality of a signal is determined relative to a predetermined range of values which relate to at least one parameter of a signal recorded from at least one selected electrode with a target quality level.

According to some exemplary embodiments, signal quality of the electrodes is determined relatively to a selected signal from a specific electrode. In some embodiments, the selected signal has a target signal peak to peak, optionally the highest signal peak to peak relative to other measured signals from other different electrodes. Alternatively or additionally, the signal quality is determined absolutely, for example relative to a predetermined reference value or range of values.

According to some exemplary embodiments, signal quality of the electrodes is determined relatively to a selected signal range from a specific electrode. In some embodiments, the selected signal has a target signal range, optionally within the range of twice the highest signal peak to peak, measured from a different electrode. Alternatively or additionally, the signal quality is determined absolutely, for example relative to a predetermined maximum and/or minimum reference value.

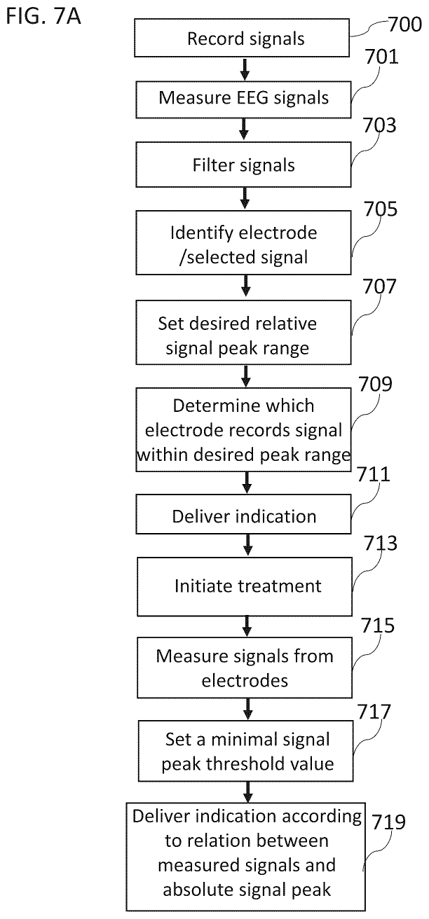

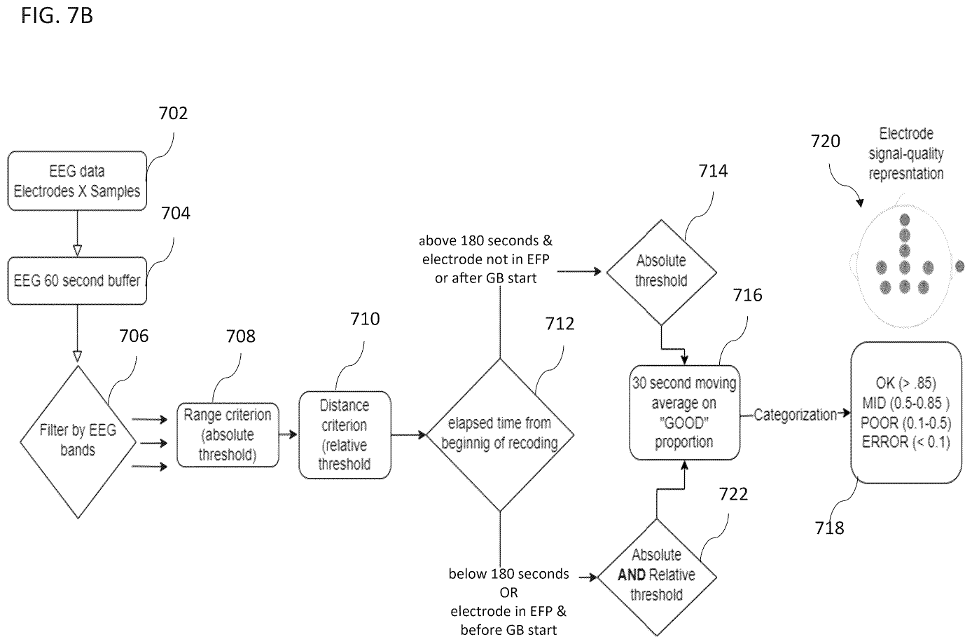

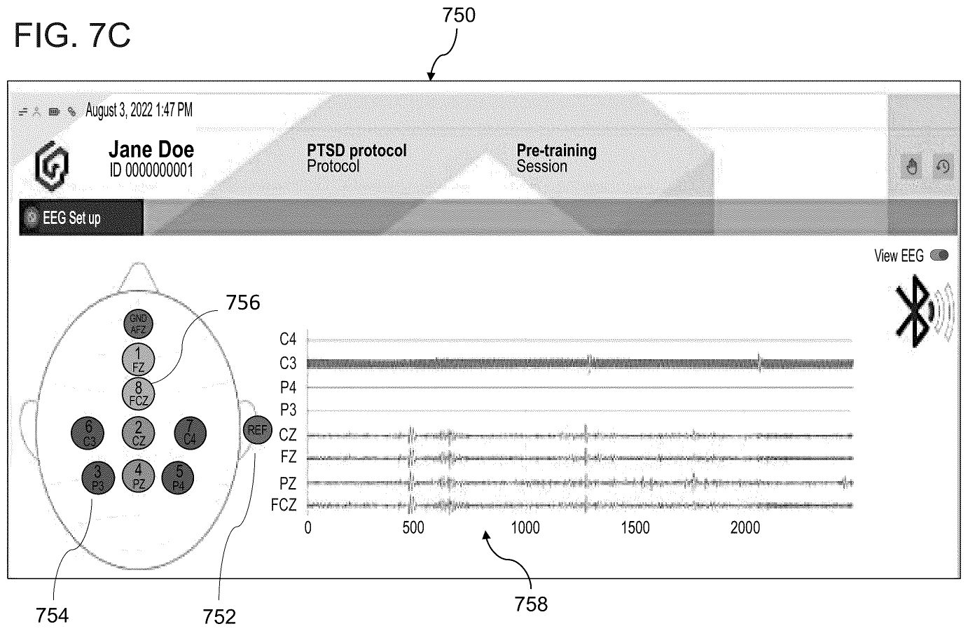

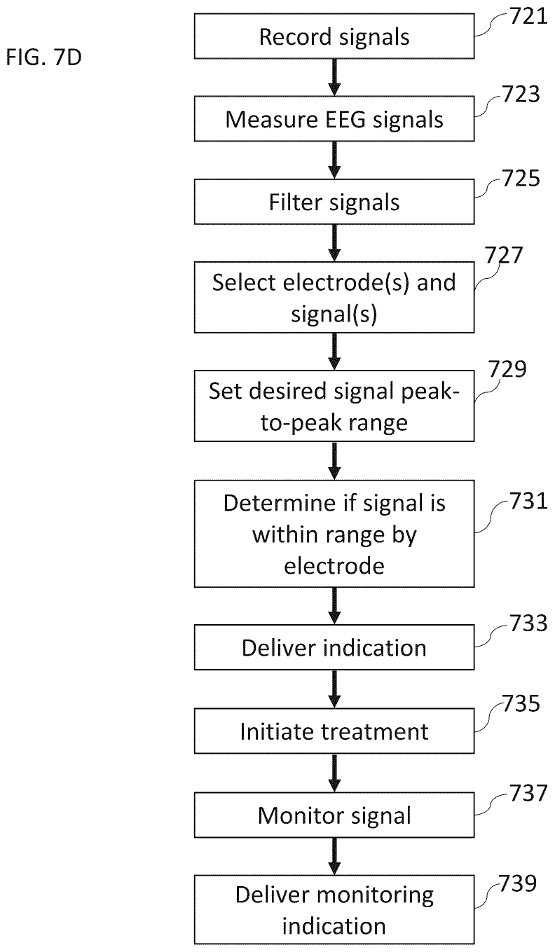

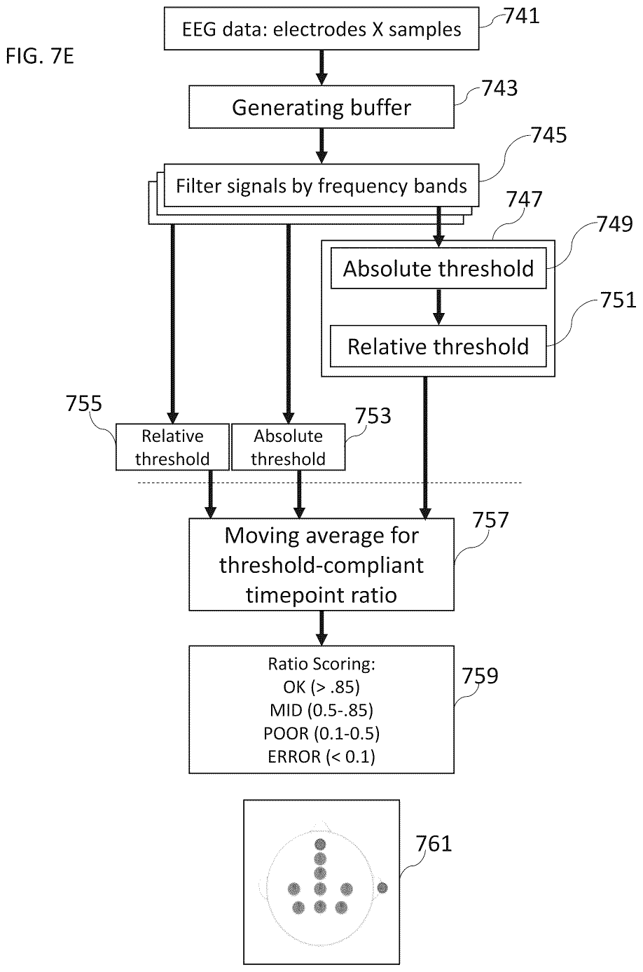

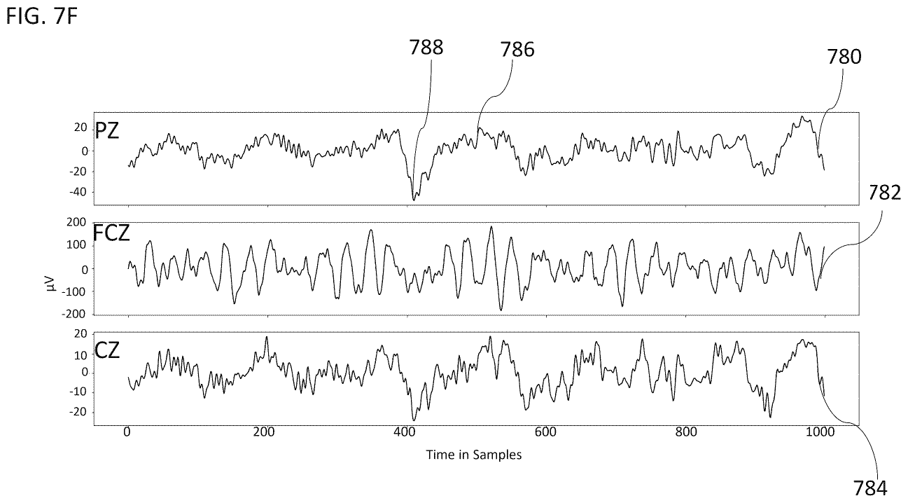

Reference is now made to FIG. 7A, depicting a general process for determining signal quality, according to some exemplary embodiments of the invention.

According to some exemplary embodiments, signals are recorded by electrodes positioned on a subject body, for example on a subject head, at block 700. In some embodiments, the signals are electrical signals recorded by electrodes, for example electrodes 316 and 318 shown in FIG. 3A.

According to some exemplary embodiments, EEG signals are measured at block 701. In some embodiments, the EEG signals are measured from the electrical signals recorded at block 700.

According to some exemplary embodiments, the EEG signals are preprocessed, at block 703. In some embodiments, the preprocessing of the EEG signals comprises filtering of the EEG signals. In some embodiments, the signals are filtered using a band-pass filter of 49 Hz-51 Hz frequencies, for example to remove line noise from the measured signals. Additionally, the filtered or the measured signals are further filtered using a 0.1 Hz-40 Hz band-pass filter, for example a 0.1 Hz-10 Hz filter, a 1 Hz-30 Hz filter, a 0.5 Hz-20 Hz filter, a 1 Hz-40 Hz filter, or any intermediate, smaller or larger range of frequencies.

According to some exemplary embodiments, an electrode which is used to record a selected signal is identified at block 705. In some embodiments, the selected signal has the highest peak value, or a target, for example a desired, peak value.

According to some exemplary embodiments, the system or a user of the system sets a range of peak values relative to the target peak value, at block 707. In some embodiments, the target range comprises peak values that are at least 50% of the target peak value, for example at least 60%, at least 70%, at least 80%, at least 90%, at least 95%, or any intermediate, smaller or larger percentage value or range value relative to the peak value of the selected signal identified at block 705.

According to some exemplary embodiments, the system determines at block 709 which electrode generates a signal which is within the target range determined at block 707.

According to some exemplary embodiments, the system generates and delivers an indication regarding the determining results, at block 711. In some embodiments, the indication includes information about at least one electrode that generates a signal that is not within the predetermined range, and/or about at least one electrode that generates a signal that is within the predetermined range. In some embodiments, the indication is a human detectable indication, optionally a visual indication and optionally a color-coded indication. In some embodiments, the indication comprises instructions how to improve the signals generated by one or more electrodes that generate signals not within the target range, so the generated signals will be within the target predetermined range.

According to some exemplary embodiments, the system initiates the NF treatment, at block 713. In some embodiments, the system initiates the NF treatment by measuring EEG signals and delivering a feedback signal to the treated subject, for example a trainee, indicating an activity or changes thereof of at least one specific brain region or at least one specific brain network based on the measured EEG signals. In some embodiments, the system starts the NF treatment after a sufficient number of electrodes generate EEG signals that are within the predetermined range that was set at block 707.

According to some exemplary embodiments, during the NF treatment, signals are measured by the electrodes, at block 715. In some embodiments, the signals, for example EEG signals, are measured by the electrodes determined at block 709 to generate a signal with a sufficient quality. According to some exemplary embodiments, a minimal signal peak threshold is set, at block 717.

According to some exemplary embodiments, during the NF treatment the system delivers an indication, at block 719 according to a relation between a measured signal peak and the minimal signal peak threshold, at block 719. In some embodiments, the system determines that the signal quality is low or insufficient, if the signal peak value is lower than the threshold value. In some embodiments, the system determines that the signal quality is good, or sufficient, if the signal peak value is higher than the threshold value. In some embodiments, the indication is a human detectable indication. In some embodiments, the indication is a visual indication. Optionally, the indication is a color coded indication.

According to some exemplary embodiments, it should be noted that a quality of a signal is assessed by determining a relation or a function between signals measured between two electrodes or between any group of electrodes, relative to each other or relative to a reference value.

Reference is now made to FIG. 7B, depicting a process for determining signal quality recorded from one or more electrodes, according to some exemplary embodiments of the invention. In some embodiments, the process allows real-time assessment of EEG (electroencephalogram) signal quality during data acquisition and optionally raises an alert when acquisition quality is low. According to some exemplary embodiments, EEG data is provided at block 702. In some embodiments, the EEG data is provided as a stream from one or more electrodes or electrodes at a predefined sample rate in a range between 50 Hz-1000 Hz, for example 100 Hz, 128 Hz, 250 Hz, 256 Hz, 500 Hz, 512 Hz, or any intermediate, smaller or larger range of sample rate frequencies. In some embodiments, the EEG data stream (for example amplitude in μV per sample) is provided from 8 electrodes at 250Hz sample rate. In some embodiments, the EEG data is provided in blocks of 1, 2, 3, 5, 10, 30, 60, 120, 180 seconds and any intermediate, smaller or larger value, to evaluate the signal quality.

According to some exemplary embodiments, the received stream is collected to an EEG Data Buffer, at block 704. In some embodiments, the buffer contains up to 100 seconds, for example up to 80 seconds, up to 60 seconds, up to 40 seconds, up to 20 seconds, up to 10 seconds, of the most recent portion of the EEG stream.

According to some exemplary embodiments, one or more pre-defined frequency band filters are applied on the EEG Data Buffer, at block 706. In some embodiments, the filters break down the EEG signal into artifact specific time-series, each of size Electrodes x Buffer length.

According to some exemplary embodiments, in order to avoid edge-effects when frequency filters are applied, the 2nd most recent single second EEG is extracted from the filtered buffer and the following steps are optionally applied:
a. For each band the selected second or a sequence of seconds (parameter), for example 3 seconds, 5 seconds or any number of seconds, is divided into two (parameter) sections and the amplitude range in each section is compared to a predefined absolute threshold that was calculated on a large sample of data, at block 708. The extracted second or the sequence of seconds, is classified as “GOOD” (1) if the range in—for a specified portion of sections—falls within the precomputed threshold values.
b. For specific frequency bands such as the power-line noise (e.g., 49-51 Hz) a relative threshold-which is an amplitude ratio between each channel to the channel that shows the lowest peak-to-peak amplitude in the time-window of interest-is added to (a), at block 710. Optionally, this can be performed within a restricted time period and applied on specific electrodes, at block 712. For example, at a specific time window of up-to 180 seconds from the beginning of the EEG recordings and for EFP model electrodes only, a second is classified as “GOOD” only if absolute and relative thresholds are met. Otherwise, the time interval is classified as “BAD”, at block 722. Optionally, in the remaining part of the EEG recording, only (a) is applied for the classification, at block 714.

According to some exemplary embodiments, the above per-second classification is then reduced to a time window in a range between 10-60 seconds, for example a time window of 30 second moving average that represent the most recent acquired signal from each channel, at block 716. In some embodiments, at every (1 second or a portion thereof or any different time period) step, the moving average is scored according to the proportion of “GOOD” time-points within the window, ranging from “OK” (above a predetermined value for example 0.85) to “ERROR” (below a predetermined value, for example 0.1), at block 718. The scoring categories can then be presented on an electrode layout visualization to guide the device operator to improve the signal of the bad channel, at block 720.

Reference is now made to FIG. 7C depicting a feedback interface which provides indications regarding the signal quality per electrode, according to some exemplary embodiments of the invention.

According to some exemplary embodiments, signal quality interface 750 comprises at least one indication, for example a color coded indication, optionally using a graphical representation of electrodes locations on a head of a subject, regarding to the signal quality per electrode, for example per electrode coupled to a head of the subject. In some embodiments, an electrode which records a signal with sufficient quality is presented in green, optionally a location of the electrode in an EEG coordinate system is presented in green, for example electrode 752. In some embodiments, an electrode which records a signal with insufficient quality is presented in red, for example electrode 754. In some embodiments, an electrode which records a signal with an intermediate quality is presented in orange, for example electrode 756.

According to some exemplary embodiments, in a visual interface, each of the electrodes is annotated for recording a signal with sufficient quality (optionally with “good” annotation), annotated for recording a signal with insufficient quality (optionally with “bad” annotation), or annotated for not recording a signal or for recording a signal with quality that is lower than a threshold for insufficient quality (optionally with “error annotation).

According to some exemplary embodiments, if all electrodes or most of the electrodes fall in the “ERROR” range (annotated as “error” optionally color coded as red), then only the ground and reference electrodes are visualized in the visual interface to the operator/supervisor, optionally with instructions and/or guidelines for prioritize treating these electrodes initially, as they optionally significantly impact the overall acquisition quality. In some embodiments, only after addressing the ground and reference electrodes should the operator attempt to enhance the signal of the remaining electrodes.

According to some exemplary embodiments, the signal quality interface comprises instructions on how to improve a signal quality recorded by one or more of the electrodes. In some embodiments, the signal quality interface comprises a graphical representation of the EEG signal recorded by each electrode, for example in a form of a graph 758 or a series of graphs.

Reference is now made to FIG. 7D depicting an additional process for determining of signal quality, according to some exemplary embodiments of the invention.

According to some exemplary embodiments, signals are recorded by electrodes positioned on a subject body, for example on a subject head, at block 721. In some embodiments, the signals are electrical signals recorded by electrodes, for example electrodes 316 and 318 shown in FIG. 3A. According to some exemplary embodiments, EEG signals are measured at block 723. In some embodiments, the EEG signals are measured from the electrical signals recorded at block 721.

According to some exemplary embodiments, the EEG signals are preprocessed, at block 725. In some embodiments, the preprocessing of the EEG signals comprises filtering of the EEG signals. In some embodiments, the signals are filtered using a band-pass filter with a frequency of about 49.9 Hz to about 50.1 Hz, for example to evaluate the line noise of the measured signals. Additionally the measured signal can be filtered using an about 2 Hz to about 40 Hz band-pass filter, for example an about 0.1 Hz-about 10 Hz filter, an about 1 Hz-about 30 Hz filter, an about 0.5 Hz-about 20 Hz filter, an about 1 Hz-about 40 Hz filter, or any intermediate, smaller or larger range of frequencies.

According to some exemplary embodiments, at least one electrode, and at least one preprocessed signal is selected and identified at block 727. In some embodiments, the selected signal is a preprocessed signal (a signal that is the result of the preprocessing performed at block 725) filtered using band-pass filter, for example as described at block 725. In some embodiments, the at least one selected electrode is the electrode which records the at least one selected preprocessed signal.

According to some exemplary embodiments, the system or a user of the system sets a target range of peak-to-peak values relative to a target peak-to-peak value, at block 729. In some embodiments, the target range is defined by a maximum and minimum peak-to-peak values within which the selected preprocessed signal identified at block 727 must remain for a specified percentage of time within the target range of peak to peak values, optionally ensuring it neither exceeds the maximum nor falls below the minimum, for example during at least 50%, at least 60%, at least 70%, at least 80%, or at least 90%, or any intermediate, smaller or larger percentage, of the time of the chosen signal.

According to some exemplary embodiments, the system determines at block 731 which electrode generates a signal which is within the target range determined at block 729.

According to some exemplary embodiments, the system generates and delivers an indication regarding the determining results, at block 733. In some embodiments, the indication includes information about at least one electrode that generates a signal that is not within the predetermined peak to peak range, and/or about at least one electrode that generates a signal that is within the predetermined peak to peak range. In some embodiments, the indication is a human detectable indication, optionally a visual indication and optionally a color-coded indication. In some embodiments, the indication comprises instructions how to improve the signals generated by one or more electrodes that generate signals not within the target range, so the generated signals will be within the target predetermined peak to peak range.

According to some exemplary embodiments, the system initiates the NF treatment, at block 735. In some embodiments, the system initiates the NF treatment by measuring EEG signals and delivering of a feedback signal to the treated subject, for example a trainee, indicating an activity or changes thereof of at least one specific brain region or at least one specific brain network based on the measured EEG signals. In some embodiments, the system starts the NF treatment only after a sufficient number of electrodes, or a predefined subgroup of electrodes generate EEG signals that are within the predetermined peak to peak range that was set at block 729. In some embodiments, the predefined group of electrodes comprise electrodes located at specific locations on a head of the treated subject, that are mandatory for recording EEG signals which indicate the activity of the at least one specific brain region or the at least one specific brain network, or changes thereof.

According to some exemplary embodiments, during the NF treatment, signals recorded by each electrode are monitored, at block 737. In some embodiments, the signals, for example EEG signals, are monitored to determine whether they meet the criteria defined at block 729. In some embodiments, the monitoring of the signal comprises monitoring a signal peak-to-peak range. In some embodiments, the monitoring comprises determining that a signal peak to peak value is within the peak to peak range defined at block 729, within a sub-range of the peak to peak range, or within a wider range relative to the defined peak to peak range.

According to some exemplary embodiments, during the NF treatment the system delivers an indication, at block 739 according to a relation determined between a measured signal and a desired target signal, at block 739. In some embodiments, the system determines that the signal quality is low or insufficient, if the monitored signal is not within a target peak to peak range, is not within a target sub-range, or is not within a derived target range, as described at block 737. In some embodiments, the system determines that the signal quality is good, or sufficient, if the monitored signal is within the target peak to peak range, or a target sub-range or any derived target range. In some embodiments, the indication is a human detectable indication. In some embodiments, the indication is a visual indication. Optionally, the indication is a color coded indication. Optionally, the treatment is paused, prior to or during the delivery of an indication about an insufficient quality of at least one signal measured by one or more of the electrodes. In some embodiments, the treatment is resumed, optionally automatically, when the signal quality is improved to a sufficient level.

According to some exemplary embodiments, it should be noted that a quality of a signal is assessed by determining a relation or a function between signals measured between two electrodes or between any group of electrodes, relative to each other or relative to a reference value.

Reference is now made to FIG. 7E, depicting an additional process for determining signal quality recorded from one or more electrodes, according to some exemplary embodiments of the invention.

In some embodiments, the process allows real-time assessment of EEG signal quality during data acquisition and optionally raises an alert when signal quality is lower than a target signal quality, optionally needed to obtain reliable measurements of activity of at least one brain region, of at least one brain network, of at least one brain system, or changes thereof.

According to some exemplary embodiments, EEG data is provided at block 741. In some embodiments, the EEG data is provided as a stream from one or more electrodes or electrodes at a predefined sample rate in a range between about 50 Hz and about 1000 Hz, for example about 100 Hz, about 128 Hz, about 250 Hz, about 256 Hz, about 500 Hz, about 512 Hz, or any intermediate, smaller or larger range of sample rate frequencies. In some embodiments, the EEG data stream (for example amplitude in μV per sample) is provided from at least one electrode or two or more electrodes, for example 2, 4, 6, 8, 10 or any intermediate or larger number of electrodes at a sample rate in a range between about 50 Hz and about 1000 Hz, for example about 100 Hz, about 128 Hz, about 250 Hz, about 256 Hz, about 500 Hz, about 512 Hz, or any intermediate, smaller or larger range of sample rate frequencies. In some embodiments, the EEG data is provided in time blocks of at least 1 second, for example 1, 2, 3, 5, 10, 30, 60, 120, 180 seconds, or any intermediate, smaller or larger value, for example to evaluate the signal quality.

According to some exemplary embodiments, the received stream is collected to an EEG Data Buffer, at block 743. In some embodiments, the buffer contains consecutive epochs of up to 100 seconds, for example up to 80 seconds, up to 60 seconds, up to 40 seconds, up to 20 seconds, up to 10 seconds, or any intermediate, smaller or larger value, of the most recent portion of the EEG stream.

According to some exemplary embodiments, one or more pre-defined frequency band filters are applied on the EEG Data Buffer, at block 745. In some embodiments, the filters break down the EEG signal into artifact specific time-series, each of size Electrodes x Buffer length, optionally, to produce multiple EEG epoch segments for different frequency bands. For example, signals are filtered using a band-pass filter with a frequency of about 49.9 Hz to about 50.1 Hz to evaluate the line noise artifacts. Alternatively or additionally, signals are filtered using a band-pass filter with a frequency of about 0.5 Hz to about 12 Hz to evaluate blink artifacts.

According to some exemplary embodiments, for example in order to avoid edge-effects when frequency filters are applied, the 2nd most recent single second EEG, optionally the second most recent EEG epoch, is extracted from the filtered buffer and the following steps are optionally applied:
a. For each band the selected second or a sequence of seconds (parameter), for example epochs that contain 3 seconds, 5 seconds or any number of seconds, is divided into two (parameter) sections and the amplitude range in each section is compared to a predefined absolute threshold value or a range of values that was optionally calculated based on a large sample of data, at block 749. The extracted second or the sequence of seconds, is classified as “GOOD” (1) if the range in—for a specified portion of sections—falls within the precomputed threshold values. For example the comparison is made for each segment's peak to peak and to the absolute threshold minimum and maximum values. For example we take the peak to peak of 2-40Hz and check if it is within the 5-100 amplitude threshold range in microvolts.
b. In some embodiments, for epochs of specific frequency bands such as the power-line noise (e.g., about 49.9-about 50.1 Hz or about 59.9-about 60.1 depending on local electric power grid frequency) optionally a relative threshold is added, at block 751. In some embodiments, the relative threshold comprises two additional criteria that are added to (a). Optionally, the filtered EEG epoch segment should satisfy one of the predetermined conditions. For example, the peak-to-peak amplitude must not exceed twice the value of the channel with the lowest peak-to-peak amplitude, and/or the filtered EEG epoch segment should fall below 30% of the range within the filter window. In some embodiments, these parameters were selected based on analysis of large datasets of EEG recordings. Optionally, satisfying the one or more predetermined conditions is performed on epochs within a restricted time period and applied per specific electrodes used for recording the EEG epochs, for example, at a specific time window of up-to 180 seconds from the beginning of the EEG recordings and per selected electrodes that are related to an EFP model for example one or more electrodes at positions C4, F7, F8, T7, T8, P8, TP9 and TP10, and/or C4, C3, Cz, P3, P4, Pz). In some embodiments, one or more seconds of an EEG is classified as “GOOD” only if absolute and relative thresholds are met. Otherwise, the EEG epoch is classified as “BAD”, at block 747. Optionally, in the remaining part of the EEG recording, only (a) is applied for the classification of each second, an average of seconds or an entire epoch as having a “GOOD” signal quality or a “BAD” signal quality, at block 753.
c. Alternatively, the relative mechanism outlined in (b) has the potential to replace the decision made in (a), enabling the system to adopt a more relaxed rather than strict approach at block 755.

According to some exemplary embodiments, the above per-second classification is then reduced to a time window, for example an EEG epoch, in a range between about 3 seconds and about 60 seconds, for example a time window of 30 second moving average that represent the most recent acquired signal from each channel (for example each electrode), at block 757. In some embodiments, for every (1 second or a portion thereof or any different time period) step, the moving average is scored according to the proportion of “GOOD” time-points within the window, ranging from “OK” (above a predetermined value for example 0.85) to “ERROR” (below a predetermined value, for example 0.1), at block 759. In some embodiments, scoring indications, for example values, categories and/or annotations are then delivered via a user interface to a subject undergoing the NF treatment and/or to a supervisor of the NF treatment. Optionally, the scoring indications are delivered using a graphical representation of an electrode layout visualization, for example to guide the device operator/supervisor to improve a signal recorded by a channel annotated as “bad”, at block 761. For example, as shown in FIGS. 7F, 3 EEG signals were recorded each by a different electrode, signal 780 recorded by an electrode at a PZ location, signal 782 recorded by an electrode at a FCZ location, and signal 784 recorded by an electrode at a CZ location, on a subject scalp. In some embodiments, the electrodes locations are according to a 10-20 coordinate system or an extended 10-20 coordinate system.

According to some exemplary embodiments, the EEG signals 780, 782 and 784 are filtered with a bandpass filter of about 2 Hz to about 40 Hz. In some embodiments, each of the signals is divided into segments, for example epochs of a predetermined time period. In some embodiments, a peak to peak value, is an absolute value of a difference between a maximal value of an amplitude, for example maximal amplitude value 786 and a minimal value of an amplitude, for example minimal amplitude value 788.

According to some exemplary embodiments, the peak to peak value or average thereof per each signal in each epoch is compared to an absolute threshold, for example an absolute amplitude threshold. Optionally, the absolute amplitude threshold is predetermined and is optionally a range of values. In some embodiments, an amplitude or an average amplitude of each signal in each epoch is compared to an absolute threshold between about 5 μV (microvolts) to about 100 μV.

For example a measured peak to peak value per each epoch or an average thereof, is for electrode PZ (at position PZ which records signal 780) is about 70, for electrode FCZ is about 200 and for electrode CZ is about 40. The measured peak to peak values are then compared to the absolute threshold of about 5 μV (microvolts) to about 100 μV. In the example, signals 780 and 784 are within the absolute threshold, whereas signal 782 has a peak to peak value that is larger than an upper limit of the threshold. Therefore only signals 780 and 784 comply with the absolute threshold.

Then, in some embodiments, compliance of the recorded signals with a relative threshold is examined, optionally compliance of only the signals that complied with the absolute threshold is examined. In the provided example, the electrode with the lowest peak-to-peak value is electrode CZ with a peak-to-peak value or an average thereof of 40. In some embodiments and in the provided example, a relative threshold is set based on the peak to peak value of the electrode with the lowest peak to peak value. In some embodiments, the relative criteria, for example the relative threshold is set to be up to 1.1 times, up to 1.5 times, up to 2 times, up to 2.5 times, up to 3 times or any intermediate, smaller o larger value of the peak to peak value of the electrode with the lowest peak to peak value.

In the example, the peak to peak value or average thereof, optionally per epoch, of the signals measured by electrode PZ is compared to the relative threshold which is based on the peak to peak value of electrode CZ (best electrode signal). For example, the relative threshold can be set to be that a peak to peak value of a signal recorded by each electrode should not exceed twice as much as the best electrode peak-to-peak (which is in our case 40*2=80 microvolts). Based on the provided example, electrode PZ passes the relative threshold.

Optionally, when the relative threshold is slightly above the lower limit of the absolute threshold, for example in a case that the relative threshold is smaller than up to 3 times, up to 4 times, up to 5 times, up to 6 times or any intermediate, smaller or larger value, of the lower limit of the absolute threshold (in the example where the lower limit is 5 μV, 5*3=15, 5*4=20 and 5*5=25, respectively), a second absolute threshold is applied. In some embodiments, the second absolute threshold is that a peak to peak value of a signal recorded by each electrode should be lower than 5%, 10%,15%, 20%, 25%, 30%, 35% or any intermediate, smaller or larger percentage value of a peak to peak value of the high absolute threshold value (for example 30% of 100 is 30). In some embodiments, a comparison of a peak to peak value to an absolute threshold and/or to a relative threshold is repeated for signal peak to peak values of each epoch and for each electrode.

According to some exemplary embodiments, each signal epoch is classified based on a level of compliance with the absolute threshold and the relative threshold and optionally the second absolute threshold, for example before and/or during the performance of a biofeedback process, for example a neurofeedback process. In some embodiments, the classification comprises calculating a compliance score for each epoch of a signal indicating the level or percentage of compliance of the signal in each epoch with the absolute and/or relative thresholds. In some embodiments, a human detectable indication, for example an audio and/or a visual indication optionally using an audio and/or a visual interface is delivered to a supervisor of the biofeedback process. For example, in some embodiments, if a compliance score is at least 0.7 (70 %), the epoch is classified as “GOOD” optionally meaning that the recorded signal in the epoch has a sufficient quality for performing the biofeedback process. For example, in some embodiments, if a compliance score is between about 0.4 (40 %) and about 0.7 (70 %), or any sub range, the epoch is classified as “MID”, optionally meaning that the recorded signal in the epoch has a quality that is less of sufficient quality. For example, in some embodiments, if a compliance score is between about 0.1 and about 0.4, the epoch is classified as “POOR”, optionally meaning that the recorded signal in the epoch is below a minimal quality for performing the biofeedback process. For example, in some embodiments, if a compliance score is lower than about 0.1, the epoch is classified as “ERROR”, optionally meaning that there is an error in recording the signal during the epoch.

According to some exemplary embodiments, the dividing into epochs, determining compliance of a signal with the absolute threshold with the relative threshold and optionally a second absolute threshold, calculating a compliance score, classifying an epoch based one the calculated compliance score, and generating a human detectable indication based on the classification is repeated using a moving window along the recorded signal, before and/or during the biofeedback process.

According to some exemplary embodiments, the generated signal quality indication, indicated or includes instructions to improve contact of the electrodes with the subject body, for example the subject scalp, when the signal quality is classified as “ERROR”, “POOR” and/or “MID”.

According to some exemplary embodiments, determining signal quality and/or that an electrode records a signal with sufficient quality, as describes in FIGS. 7A-7F is performed by system 302 described in FIG. 3A, control circuitry 310 and/or any control circuitry which is part of the system 302 or with a NF device, as described herein in the patent application.

### Exemplary NF Training Expert Interfaces

According to some exemplary embodiments, during a NF treatment session, an expert, for example a supervisor receives indications from the NF system regarding the progress of the NF treatment session. Optionally, the NF system delivers information to the expert regarding a state, for example a mental and/or cognitive state of the subject. In some embodiments, the NF training expert interfaces comprise a visual indication of the audio/visual interface presented by the NF system to the subject, and/or an indication regarding changes in the EFP signal, optionally relative to a reference or a baseline value, and optionally in the form of graph.

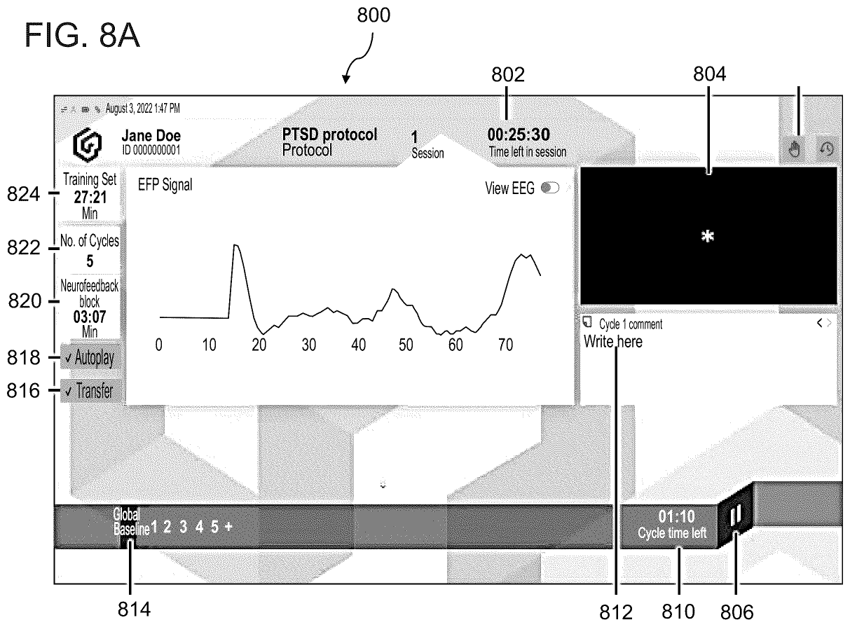

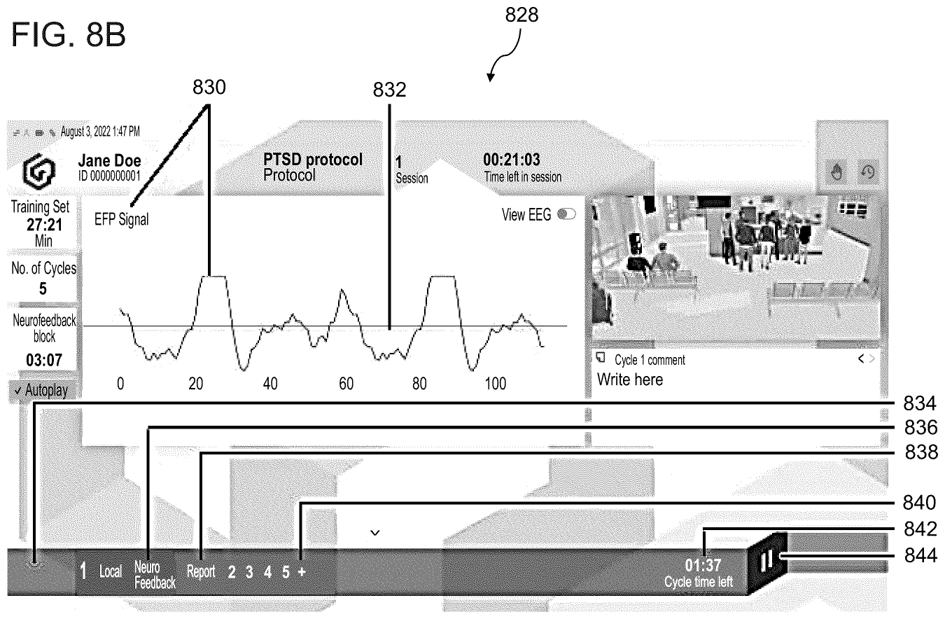

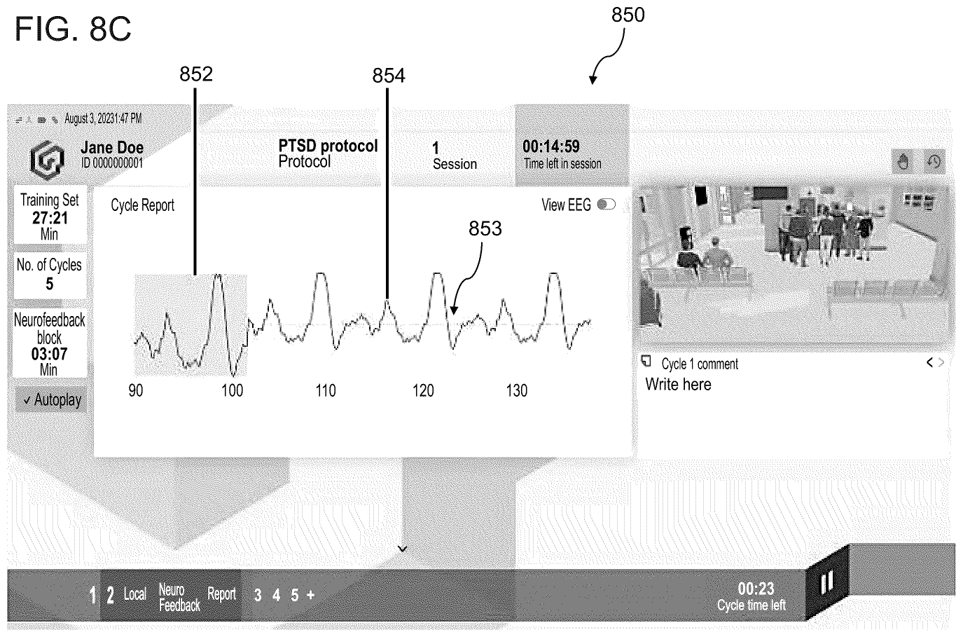

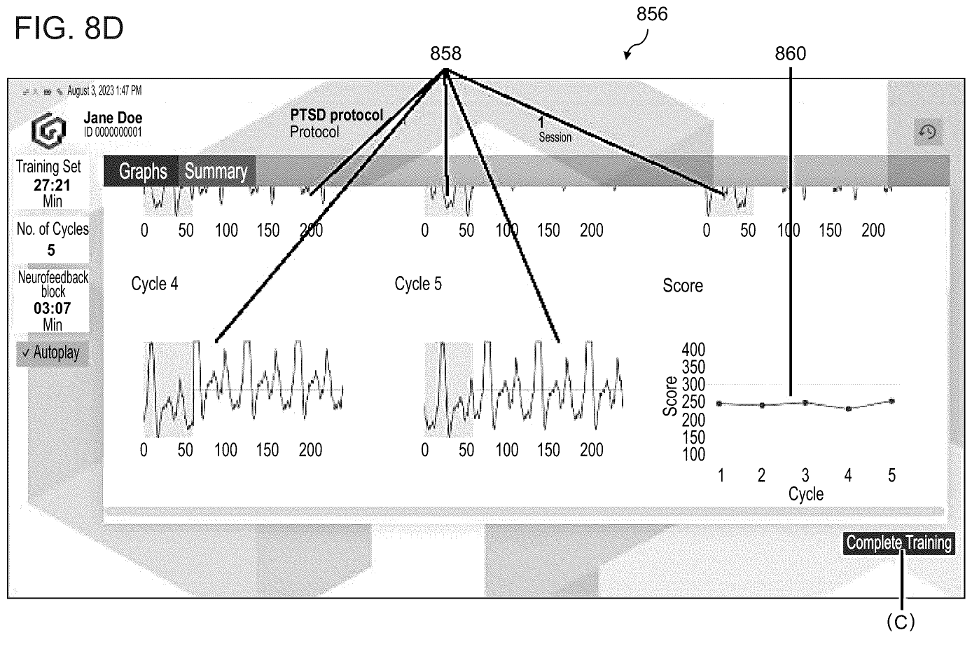

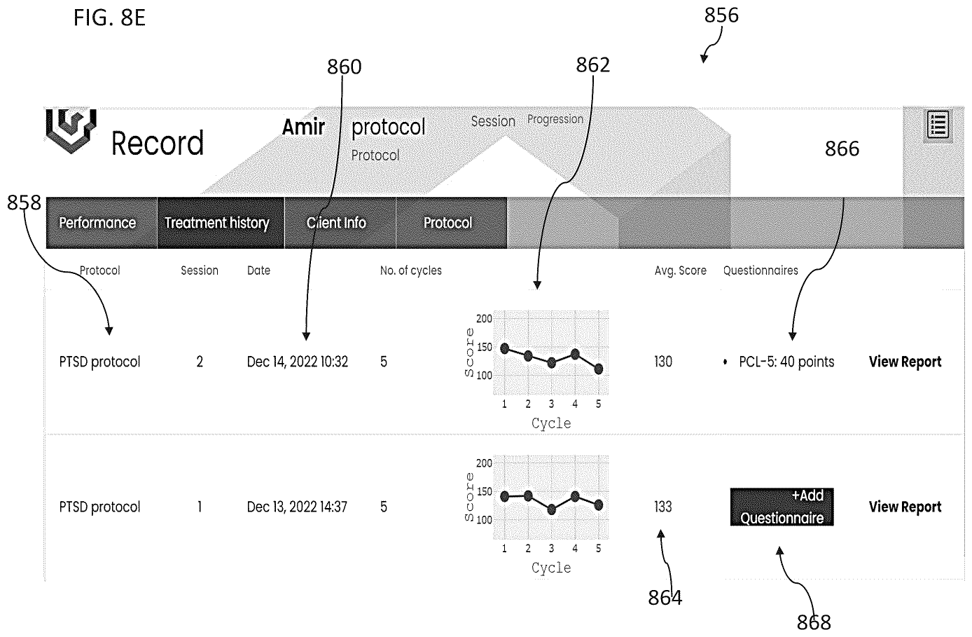

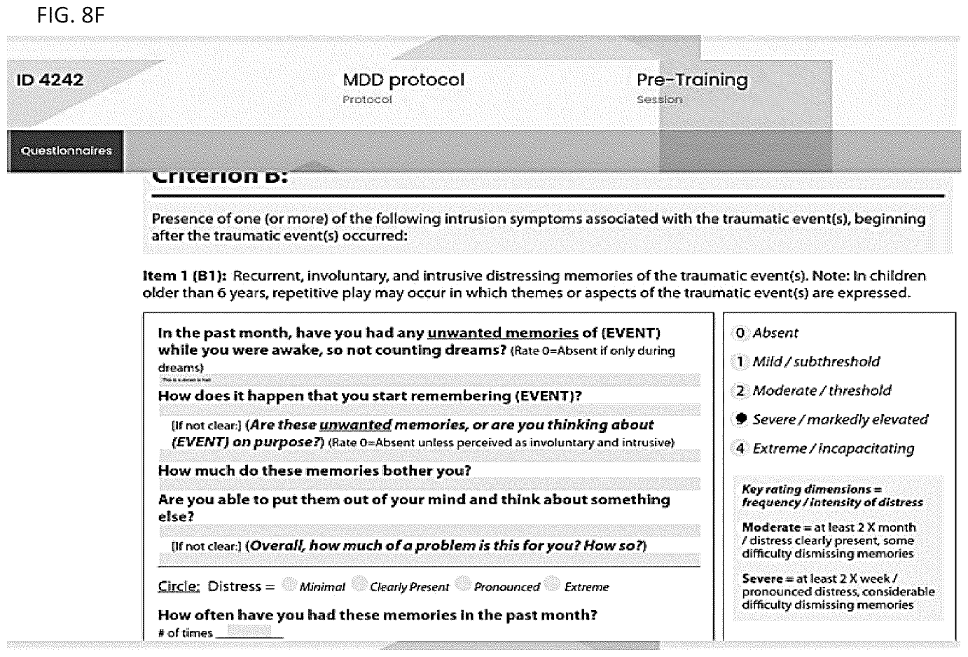

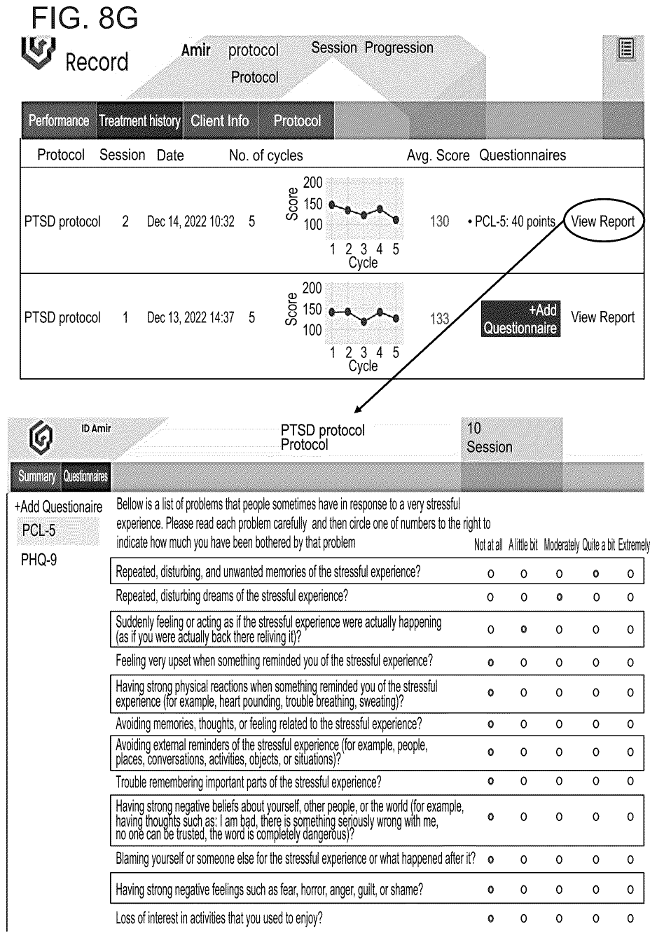

Reference is now made to FIG. 8A, depicting a global baseline expert interface, according to some exemplary embodiments of the invention.

According to some exemplary embodiments, a global baseline interface 800 comprises one or more of, an indication 802 regarding the time left in the overall session or in this specific session, an indication 804, for example a graphical representation or an actual view of the interface displayed to the trainee, optionally in real time or in delay, a pause/play button 806, an indication 810 regarding the time left for the current cycle, an indication 812 which includes comments, an indication 814 regarding the name of the current cycle, a transfer indication 816 which indicates if the training cycle or training session incudes a transfer cycle in which feedback is not delivered to the subject while he subject is presented with an audio/visual interface, for example a dynamic audio/visual interface, an auto play indication indicates that the training proceeds from cycle to cycle, an indication 820 regarding the duration of the neurofeedback blocks in all cycles of current training, an indication 822 regarding the number of active NF cycles of the current training session, and an indication 824 regarding the overall time set for the training cycle.

Reference is now made to FIG. 8B, depicting an expert NF cycle interface, according to some exemplary embodiments of the invention.

According to some exemplary embodiments, a NF cycle interface 828 comprises an indication 830 describing changes in a calculated EFP signal, or an EEG signal, during the NF cycle, optionally relative to a calculated baseline, for example a local baseline. Optionally, the EFP signal indication 830 is displayed next to the baseline indication 832, for example in the form of a graph, optionally a continuous graph.

According to some exemplary embodiments, the NF cycle interface 828 further includes at least one of, a cycle control panel 834 indication optionally displays information about the current training (for example, the numbers of the cycles in it) and tools for controlling it, a current block indication 836: the current cycle is expanded to display the names of its blocks, and the name of the current block is in bold; as a block progresses, more and more of its background color optionally changes to red (from red-grey), a future cycle indication 838, which optionally displays the number of each future cycle, an add cycle button 840 to allow adding a cycle after the protocol's default Cycles (an expert can add a Cycle at any time after the start of the Global Baseline and before the end of the currently-defined last Cycle), a cycle clock indication 842, and a pause/play button 844. Reference is now made to FIG. 8C, depicting an expert NF cycles report interface, according to some exemplary embodiments of the invention.

According to some exemplary embodiments, a NF cycle report interface, for example interface 850 comprises a graphical representation, for example as a graph, of changes in a calculated EFP signal during a local bassline session 852 and during a NF training cycle 854. In some embodiments, the graphical representation is displayed relative to a graphical representation of a baseline or a reference value, for example a local baseline 853. In some embodiments, the NF cycle report interface 850 is displayed to the expert during a rest block of a NF cycle, for example rest block 628 shown in FIG. 6B. Optionally, the NF cycle report interface comprises at least one indication of a calculated progress score.

According to some exemplary embodiments, the calculated progress score indicates a progress of the subject during the NF cycle, for example in an ability of the subject to module the activity of the at least one brain region or the at least one brain network, based on the changes in the EFP (or EEG) signal during the NF cycle. In some embodiments, the NF system provides suggestions how to proceed with the NF treatment session and/or with the overall NF treatment based on the calculated progress score.

According to some exemplary embodiments, the progress score is calculated according to a central tendency measure of the calculated EFP signal when reaching a desired value or when the calculated EFP signal is within a desired range of vales. Alternatively or additionally, the progress score is calculated according to the time the calculated EFP signal is higher or lower than a predetermined value, and/or according to the time the EFP signal is within the desired range of values, during the NF cycle. Optionally, the progress score, for example the NF cycle progress core is displayed in the interface 850.

Reference is now made to FIG. 8D, depicting a NF session report interface, according to some exemplary embodiments of the invention.

According to some exemplary embodiments, the NF session report interface, for example interface 856 comprises an indication 858, optionally a graphical representation of each of the NF cycles performed by the subject during the NF treatment session. Additionally, the interface 856 comprises an indication 860 regarding changes in a progress score calculated per each NF cycle during the NF treatment session. In some embodiments, the NF system modifies at least one parameter of the NF treatment session based on the changes in the progress score. For example, if the changes in the progress score, or a central tendency measure of the progress score are not sufficient, then the NF system precedes a following NF treatments session. Alternatively or additionally, the NF system modifies an interface, for example an audio/visual interface displayed to the subject during at least one NF cycle based on changes 860 in the progress score. In some embodiments, the NF system delivers suggestions to modify at least one parameter of the NF session treatment based on the changes in the progress score and/or a central tendency measure of the progress score. In some embodiments, the progress score is calculated by an average EFP difference between the NF cycle and the local baseline median EFP, standardized by local baseline inter-quantile-range or other statistical grouping.

Reference is now made to FIG. 8E, depicting a subject, for example a trainee NF treatment summary interface, according to some exemplary embodiments of the invention.

According to some exemplary embodiments, a trainee NF treatment summary interface, for example interface 856, comprises a summary of all the NF treatment sessions delivered to a subject, for example to trainee. Optionally, the interface 856 comprises an indication regarding at least one planned NF treatment session. In some embodiments, the interface 856 comprises for each NF treatment session, at least one of, an indication 858 regarding a NF protocol used in the NF treatment session, an indication 860 regarding the date of the NF treatment session, an indication 862, for example in a form of a graphical representation, of the changes in a progress score during NF cycles performed in the NF session, an indication 864 of a central tendency measure of the progress score per the NF session, an indication 866 regarding an assessment questionnaire and optionally a score of the questionnaire performed by the subject. Optionally, the interface 856 comprises a reminder indication 868 to perform an assessment questionnaire, for example a specific assessment questionnaire.

According to some exemplary embodiments, for example as shown in FIG. 8F, the questionnaire is filled using a user interface of the NF system. Optionally, the NF system is configured to calculate a score of the assessment questionnaire based on the answers filled in the electronic form of the questionnaire. In some embodiments, the questionnaire calculated score is displayed in 866.

According to some exemplary embodiments, the NF system is configured to calculate a subject state score based on the calculated questionnaire score or scores of different questionnaires or of the same questionnaire over time. In some embodiments, for example as shown in FIG. 8G, the memory of the NF system includes electronic forms of one or more assessment questionnaires, for example a PTSD Checklist for DSM-5 (PCL-5) electronic form, a Patient Health Questionnaire-9 electronic form, the Clinician-Administered PTSD Scale for DSM-5 (CAPS-5) and/or parameters or domains of the CAPS-5, and a Patient Health Questionnaire-9 (PHQ-9). Alternatively or additionally, the questionnaires comprises the Hamilton Depression Rating Scale or any derivative, the Snaith-Hamilton Pleasure Scale (SHAPS) or any derivative, the Montgomery-Asberg Depression Rating Scale (MADRS) or derivatives, the Patient Health Questionnaire (PHQ) or derivatives, the Clinical Global Impression (CGI) or derivatives. Alternatively or additionally, the questionnaires comprises the Adult Attention deficit hyperactivity disorder (ADHD) ADHD Investigator Symptom Rating Scale (AISRS) or derivatives, the Adult ADHD Self-Report Scale (ASRS) or derivatives, the Test of Variables of Attention (TOVA), or the Patient Health Questionnaire (PHQ).

According to some exemplary embodiments, a memory of a device of the subject, for example a personal device 214 comprises the one or more electronic forms of the assessment exams.

According to some exemplary embodiments, the NF system and/or a software application in the memory of the personal device is configured to allow filling of the electronic forms, automatically calculate a score of the assessment exam and/or optionally to deliver suggestions to the subject and/or the expert based on the calculated score of the assessment exam.

### Exemplary Relation Between Subject State and NF Treatment Progress

According to some exemplary embodiments, during NF treatment, for example during at last one NF treatment session, a state of the subject, for example a clinical, a mental and/or a cognitive state is determined, for example as described at blocks 106, 450, 456, 483, 484, 502 and 512. Additionally, a progress of the subject during at least one NF treatment session is determined, for example as described in 862, 864. In some embodiments, the NF system determines, for example calculates, a relation between the determined subject progress and the determined state of the subject. In some embodiments, the NF system delivers at least one indication to the expert and optionally to the trainee about the determined relation. In some embodiments, the determined relation is used to evaluate a current success of the NF treatment or to predict a future success of the NF treatment by either the system or a healthcare professional (HCP). Optionally, at least one parameter of the NF treatment or the NF treatment session is modified based on the determined relation.

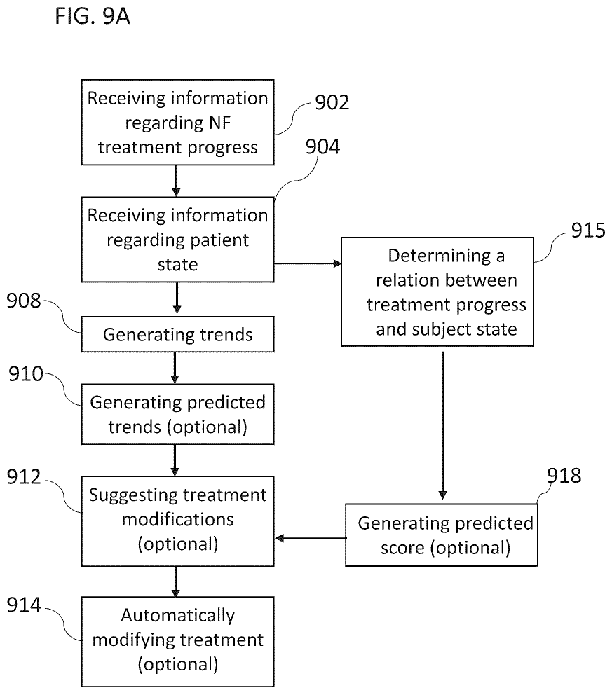

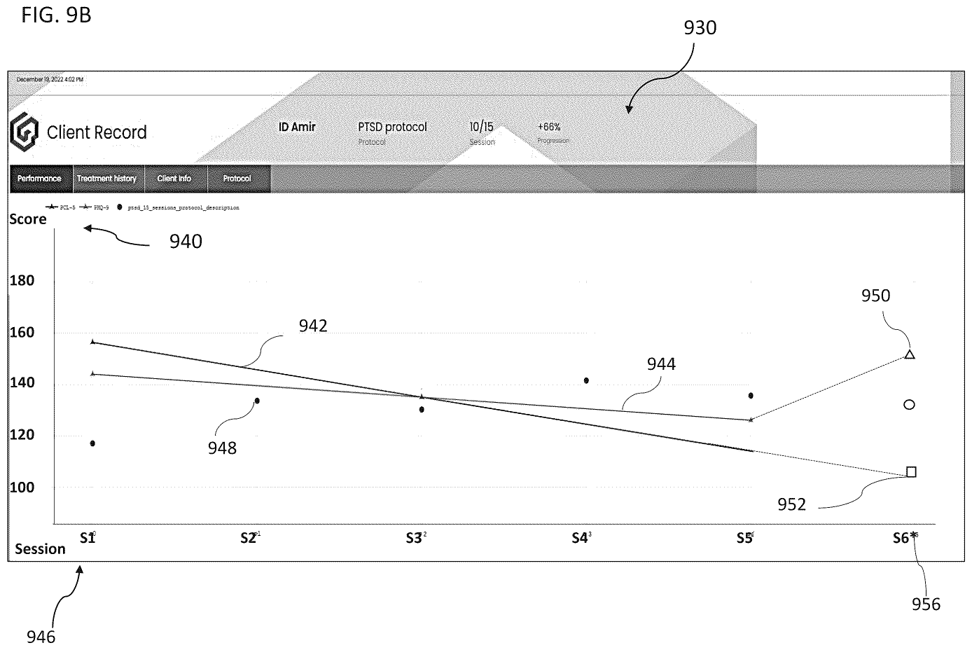

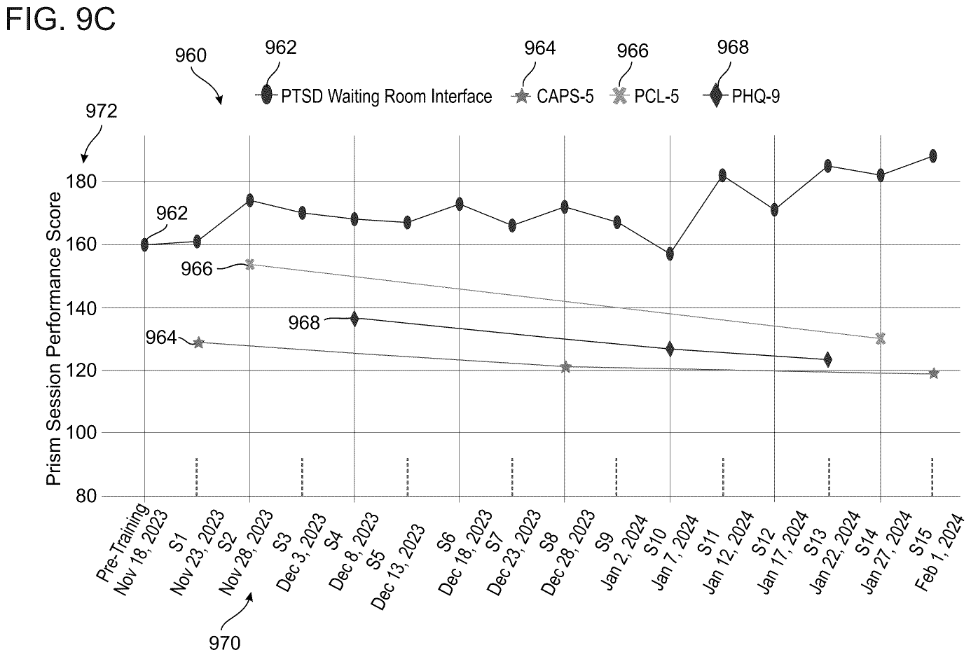

Reference is now made to FIG. 9A depicting a method for determining a relation between NF treatment progress and a state of the subject, according to some exemplary embodiments of the invention.

According to some exemplary embodiments, information regarding progress of a subject in a NF treatment is received at block 902. In some embodiments, the information includes a score indicating the subject progress in modifying the activity of at least one brain region and/or at least one brain network or a biomarker thereof in a NF treatment session, for example in at least one cycle of active NF, for example cycle 626. In some embodiments, the score is score 864 shown in FIG. 8E. In some embodiments, the progress score is calculated by an average EFP difference between the NF cycle and the local baseline median EFP, standardized by local baseline inter-quantile-range or other statistical grouping. In some embodiments, the progress score is a central tendency measurement of a difference between an EFP signal measured during an active NF cycle and a median or any other central tendency measurement of a baseline or a EFP signal measured for example during global baseline or local baseline. In some embodiments, the progress score is a value indicating the ability of the subject to modify the at least one brain region and/or the at least one brain network or a biomarker thereof, when applying a specific strategy and/or when engaging a specific audio and/or visual interface, for example interface 604. Optionally, the score is based on changes in the measured EEG or EFP signal during the NF cycle.

According to some exemplary embodiments, information regarding a patient state is received at block 902. In some embodiments, the information is based on the determined state of the subject, for example as described at blocks 106, 450, 456, 483, 484, 502 and 512, in FIGS. 1, 4B, 4C, and 5.

According to some exemplary embodiments, separate trends showing changes in the subject state and in the subject progress are generated at block 908. In some embodiments, the generated trends are displayed to an expert, for example to the supervisor of the NF treatment. In some embodiments, the trends or a relation between the trends is used to determine a subject mental state or a cognitive state and optionally plan or determine following steps of at least one of, a NF cycle, a NF session, entire NF therapy, or a different therapy, for example a psychotherapy and/or a pharmacotherapy in the subject.

According to some exemplary embodiments, predicted trends of the subject state and of the subject progress are optionally generated, at bock 910. In some embodiments, the predicted trends describe a change in the subject state and in the subject progress in the future, for example following at least one NF treatment session and/or after at last one week or at least one month. In some embodiments, the generated predicted trends are optionally displayed to the expert.

According to some exemplary embodiments, the NF system optionally suggests one or more modifications to the NF treatment, based on the generated trends and/or based on the predicted trends, at block 912. In some embodiments, in case the progress trend shows mild progress and the state trend shows that the subject state is lower/higher than a desired state, then the NF system provides instructions to continue with the current NF treatment setting and update the status after at least one NF treatment session. Alternatively or additionally, in case the progress trend shows mild progress and the state trend shows that the subject state is lower and/or higher than a desired state, then the NF system provides instructions to perform at last one subject state assessment after a specific time period following a NF treatment session and/or in a beginning of the next NF treatment session.

According to some exemplary embodiments, in case the progress trend shows significant progress and the state trend shows that the subject state is higher and/or lower than a desired state, then the NF system provides instructions to continue with the current treatment setup, or to delay the next NF treatment session, or to modify at least one parameter of the next NF treatment session, for example to increase a difficulty level of the NF treatment session and/or to shorten the NF treatment session, and/or instructions to change a regimen of standard of case (SOC) modalities, for example reduce pharmacotherapy dosage/frequency.

According to some exemplary embodiments, in case the progress trend shows very low subject progress and the state trend shows that the subject state is lower than a desired state, then the NF system provides instructions to at least one of, shorten the time to the next NF treatment session, use a different audio/visual interface, instruct the subject to use a different strategy when engaging the audio/visual interface, stop the NF treatment, stop or start a different treatment for example a drug treatment, modify a different treatment delivered to the subject for example modify a drug administration regime of a drug treatment, to modify the intensity of treatment in regard to time between sessions or drug dosage, or modify the duration of the section of the treatment such as the global baseline, local baseline, and the NF sections.

According to some exemplary embodiments, the NF system automatically modifies the NF treatment, based on the generated trends and/or the predicted trends, at block 914. In some embodiments, the NF system automatically modifies at least one parameter of the NF treatment, for example shortens or prolongs duration between NF treatment sessions, reschedules subject assessment sessions, reschedules NF treatment sessions, modifies or replaces an audio/visual interface and/or modifies the number and/or duration of active NF cycles.

According to some exemplary embodiments, a relation between treatment progress and the patient state is determined at block 915. In some embodiments, determining a relation comprises calculating a combined score based on the treatment progress score and the patient state, for example a patient state score. In some embodiments, the combined score is calculated by assigning weights to each of the calculated score of the patient state score and the treatment progress score. In some embodiments, the combined score is a score indicating a matching degree between the NF treatment and a specific subject. In some embodiments, the combined score is a score indicating how well the NF treatment having specific parameter values, fits a specific subject.

According to some exemplary embodiments, a predicted score is optionally generated at block 918. In some embodiments, the predicted score indicates a matching degree between the NF treatment and a specific subject in the future, for example when modifying one or more parameters of the NF treatment, for example to better personalize the NF treatment to a specific subject.

According to some exemplary embodiments, the NF system provides suggestions at block 912 how to personalize the NF treatment for a specific subject, for example based on the predicted score generated at block 918 and/or a score calculated at block 915.

According to some exemplary embodiments,, the NF system automatically modifies the treatment, in order to better personalize the NF treatment for a specific subject, at block 914. In some embodiments, the NF system automatically modifies at least one parameter of the NF treatment, at block 914.

Reference is now made to FIG. 9B, depicting a graphical interface showing a relation between changes of a score of a least one assessment questionnaire indicating a state of a subject, and changes in a progress score at different time points during a NF treatment, according to some exemplary embodiments of the invention.

According to some exemplary embodiments, the NF system is configured to display in a graphical interface, for example a subject status interface, a relation between a state of the subject or indication thereof, and a progress score or indication thereof, optionally as trend lines in at least one graph. In some embodiments, a subject status interface, for example interface 930 comprises a graph showing a change in a score 940 of at least one assessment questionnaire in a form of a trend line, for example line 942 representing changes in PCL-5 score, and line 944 representing change in a score of PHQ-9, between different NF treatment sessions 946. Additionally, the interface 930 displays a change in a progress score 948 as measured at an end of each NF treatment session. In some embodiments, the interface 930 displays a predicted score, for example a predicted PCL-5 score 950, a predicted PHQ-9 score 952, a predicted progress score 954, and a predicted mental and/or cognitive state of the subject, in at least one planned NF treatment session, for example session 956.

According to some exemplary embodiments, the NF system provides suggestions to the expert based on the relation between the changes in a score of the assessment questionnaire, and the changes in a progress score, in already completed NF treatment sessions, and optionally also based on a predicted relation, for example based on predicted progress score and predicted assessment questionnaire scores.

Reference is now made to FIG. 9C, depicting a graphical interface showing a summary of a progress of a subject in a NF treatment together with changes in one or more mental state exams, during the NF treatment, according to some exemplary embodiments of the invention.

According to some exemplary embodiments, a system, for example system 302 shown in FIG. 3A, generates an indication to a supervisor of a NF treatment with information about a progress of a subject undergoing the NF treatment, for example a patient or a trainee, in the NF treatment, and with information about a mental state, for example a cognitive state of the subject during the NF treatment. In some embodiments, the indication comprises visual interface optionally a graphical interface, for example graphical interface 960. In some embodiments, the indication includes information about a change in a score calculated by the system 302 which indicates success of the subject in modifying a feedback signal in a desired direction and/or in an ability of the subject to modify activity of at least a portion of a brain, for example at least one brain region, at least one brain network, and/or at least one brain system. In some embodiments, the indication includes information about a change in the feedback signal and/or a change in brain activity, during the NF treatment.

According to some exemplary embodiments, the graphical interface 960 provides a visual indication, optionally in a form of a graph 962, showing a change in a progress score of the NF treatment during a NF treatment which includes several treatment sessions 970. In some embodiments, the graph 962 is based on a progress score calculated by the system before and/or after each treatment session. In some embodiments, the progress score is an objective score optionally calculated automatically by the system 302.

Additionally and optionally in parallel, the graphical interface provides a visual indication, for example in the form of graphs 964, 966 and 968, with regard to changes in a mental state of the subject as measured using one or more mental state exams, for example questionnaires. In some embodiments, the graphs 964, 966 and 968 show a change in CAPS-5, PCL-5 and PHQ-9, respectively, during the NF treatment, and optionally in parallel and in the same graphical interface next to the indication of graph 962 showing a change in a progress score of the NF treatment.

A potential advantage of presenting both an objective score indicating progress of the subject in a NF treatment next to an indication showing changes in a mental state of the subject may be to allow a supervisor of the NF treatment to monitor an actual effect of the NF treatment on a mental state of the subject during the NF treatment and at an end of the NF treatment, for example based on a correlation between changes in the NF treatment progress and changes in the mental state of the subject. In some embodiments, the monitoring may allow the supervisor to modify the NF treatment or an overall treatment provided to the subject if there is no correlation and/or if there is no change in a mental state of the subject. Alternatively, the monitoring may allow a supervisor to stop the NF treatment or to combine the NF treated with at least one additional treatment, for example drug treatment, CBT treatment, behavioral treatment, psychological treatment, a different NF treatment or biofeedback treatment, or any other treatment, in order to improve a mental state of the subject.

An additional potential advantage of presenting both an objective score indicating progress of the subject in a NF treatment next to an indication showing changes in a mental state of the subject may be to allow a clinician treating the subject and/or health providers, health maintenance organizations (HMO's), and/or insurance companies, to monitor a mental state of a subject and/or an effect of the NF treatment on the mental state of the subject using a single interface and optionally to stop the NF treatment, to modify the NF treatment, and/or to modify an overall treatment provided to the subject based on the information provided by the interface.

As used herein with reference to quantity or value, the term “about” means “within ±20 % of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc. ; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for delivery of a neurofeedback (NF) process to a subject, comprising:
delivering at least one session of a NF process to a subject for teaching said subject to modify activity of at least one specific brain region and/or at least one specific brain network, generating a progress indication indicating an ability of said subject to modify said activity of said at least one brain region or said at least one brain network during said at least one session;
determining a state of said subject after completing said at least one NF session, wherein said state of said subject comprises at least one of, a mental state, a behavioral state, and/or a cognitive state of said subject;
generating a subject state indication based on said determined state;
displaying said progress indication and said subject state indication in a shared visual interface.

2. The method of claim 1, wherein said progress indication and said subject state indication are displayed side by side in said shared visual interface.

3. The method of claim 1, wherein said generating a progress indication comprises calculating at least one progress score indicting said progress of said subject, and at least one subject state score indicating a state of said subject, and wherein said displaying comprises displaying said progress score and said at least one subject state score in said shared visual interface.

4. The method of claim 3, wherein said calculating comprises calculating said at least one progress score based on signals measured from the subject brain during said at least one NF session, wherein said signals indicate activity of said at least one specific brain region and/or of said at least one specific brain network.

5. The method of claim 4, wherein said signals comprise EEG signals.

6. The method of claim 3, wherein said delivering comprises delivering at least 2 sessions of said NF process to said subject, and wherein said displaying comprises displaying changes in said at least one progress score and changes in said at least one subject state score during said NF process in said shared visual interface.

7. The method of claim 3, comprising determining a correlation between said calculated progress score and said at least one subject state score, and wherein said displaying comprises displaying said determined correlation.

8. The method of claim 7, comprising modifying said NF treatment delivered to said subject based on said determined correlation.

9. The method of claim 1, comprising receiving information about a state of said subject after completing said at least one NF session, wherein said information comprises results of at least one assessment questionnaire, self-reported information, and/or input from an expert about the state of the subject, and wherein said state of said subject is determined based on said received information.

10. The method of claim 9, wherein said at least one assessment questionnaire comprises at least one of, a Clinician-Administered PTSD Scale for DSM-5 (CAPS-5) and/or parameters or domains of the CAPS-5 or derivatives thereof, a PTSD Checklist for DSM-5 (PCL-5) or derivatives thereof, a Patient Health Questionnaire-9 (PHQ-9) or derivatives thereof, a Hamilton Depression Rating Scale or derivatives thereof, a Snaith-Hamilton Pleasure Scale (SHAPS) or derivatives thereof, a Montgomery-Asberg Depression Rating Scale (MADRS) or derivatives thereof, a Patient Health Questionnaire (PHQ) or derivatives thereof, a Clinical Global Impression (CGI) or derivatives thereof, an Adult Attention deficit hyperactivity disorder (ADHD) ADHD Investigator Symptom Rating Scale (AISRS) or derivatives thereof, an Adult ADHD Self-Report Scale (ASRS) or derivatives thereof, a Test of Variables of Attention (TOVA) or derivatives thereof, a Patient Health Questionnaire (PHQ) or derivatives thereof, an Alzheimer's Disease Assessment Scale-Cognitive Subscale (ADAS-Cog) or derivatives thereof, a mini-mental state examination (MMSE) or Folstein test or derivatives thereof.

11. The method of claim 1, comprising: diagnosing said subject with a mental disorder or at least one symptom thereof, prior to said delivering, wherein said mental disorder comprises at least one of, a stress disorder, post-traumatic stress disorder (PTSD), depression, Attention deficit hyperactivity disorder (ADHD), and/or substance use disorder (SUD).

12. A system comprising:
a neurofeedback (NF) device configured to deliver at least one session of a NF treatment to a subject for teaching said subject to modulate an activity of at least one specific brain region or at least one specific brain network, comprising:
a control circuitry;
a memory storing at least one protocol of said NF treatment, a progress indication indicating a progress in an ability of said subject to modulate an activity of said at least one specific brain region or said at least one specific brain network, during said at least one session, and a subject state indication with information about a state of the subject following said at least one session and during the NF treatment, wherein said state of the subject comprising at least one of, a mental state, a behavioral state, and/or a cognitive state of said subject;
a user interface in communication with said NF device, configured to generate and display at least one visual interface, wherein said control circuitry of said NF device is configured to signal said user interface to generate and deliver said at least one visual interface with said progress indication and said mental state indication.

13. A system according to claim 12, wherein said memory stores electrical signals measured from the subject brain during said at least one session, wherein said measured electrical signals indicate activity of said at least one specific brain region or said at least one specific brain network, and wherein said control circuitry generates said progress indication based on said electrical signals.

14. A system according to claim 13, wherein said electrical signals comprise EEG signals.

15. A system according to claim 12, wherein said control circuitry is configured to receive input signals with said information about said mental state of said subject, wherein said input signals comprise results of at least one assessment questionnaire, self-reported information, and/or input from an expert or a supervisor about said state of the subject.

16. A system according to claim 15, wherein said at least one assessment questionnaire comprises at least one of, a Clinician-Administered PTSD Scale for DSM-5 (CAPS-5) and/or parameters or domains of the CAPS-5 or derivatives thereof, a PTSD Checklist for DSM-5 (PCL-5) or derivatives thereof, a Patient Health Questionnaire-9 (PHQ-9) or derivatives thereof, a Hamilton Depression Rating Scale or derivatives thereof, a Snaith-Hamilton Pleasure Scale (SHAPS) or derivatives thereof, and a Montgomery-Asberg Depression Rating Scale (MADRS) or derivatives thereof.

17. A system according to claim 12, wherein said control circuitry is configured to determine a correlation between said progress indication and said subject state indication, and to signal said user interface to display said determined correlation.

18. A system according to claim 17, wherein said control circuitry is configured to signal said user interface to generate a human detectable indication with a suggestion to modify said NF treatment based on said determined correlation.

19. A system according to claim 12, wherein said control circuitry is configured to generate a trend of a change in said progress indication, and a trend of a change in said subject state indication over a selected time period of said NF treatment and/or predictions thereof, and to signal said user interface to display said generated trend of said change in said progress indication and said trend of said change in said subject state indication, and/or of said predictions thereof.

20. A system according to claim 12, wherein said control circuitry is configured to signal said user interface to generate a human detectable indication with a suggestion to modify said NF treatment based on said subject state indication and/or of said information about a state of the subject stored in said memory.
